(19) United States
(12) Patent Application Publication
(10) Pub. No.: US 2023/0205083 A1
(43) Pub. Date: Jun. 29, 2023

(54) SALT COMPOUND, RESIST COMPOSITION AND PATTERNING PROCESS

(71) Applicant: Shin-Etsu Chemical Co., Ltd., Tokyo (JP)

(72) Inventors: Takayuki Fujiwara, Joetsu-shi (JP); Tomomi Watanabe, Joetsu-shi (JP); Masayoshi Sagehashi, Joetsu-shi (JP)

(73) Assignee: Shin-Etsu Chemical Co., Ltd., Tokyo (JP)

(21) Appl. No.: 18/068,564

(22) Filed: Dec. 20, 2022

(30) Foreign Application Priority Data
Dec. 27, 2021 (JP) 2021-212355

(51) Int. Cl. G03F 7/004; G03F 7/039; G03F 7/038; C07C 381/12; C07C 65/21; C07D 333/76; C07D 327/08; C07C 69/753; C07C 311/51; C08F 220/18

(52) U.S. Cl. CPC G03F 7/0045; G03F 7/039; G03F 7/038; C07C 381/12; C07C 65/21; C07D 333/76; C07D 327/08; C07C 69/753; C07C 311/51; C08F 220/1818; C08F 220/1806; C08F 220/1808; C07C 2602/42

(57) ABSTRACT

A salt having formula (1) or (2) serving as an acid diffusion inhibitor is provided as well as a resist composition comprising the acid diffusion inhibitor. When processed by lithography, the resist composition exhibits a high sensitivity, and excellent lithography properties such as CDU and LWR.

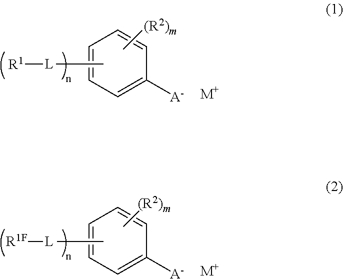

### CROSS-REFERENCE TO RELATED APPLICATION

This non-provisional application claims priority under 35 U.S.C. § 119(a) on Patent Application No. 2021-212355 filed in Japan on Dec. 27, 2021, the entire contents of which are hereby incorporated by reference.

### TECHNICAL FIELD

This invention relates to a salt compound, a resist composition, and a pattern forming process.

### BACKGROUND ART

To meet the recent demand for higher integration and operating speeds in LSIs, further miniaturization of the pattern rule is desired. The requirement to form resist patterns of high resolution necessitates not only to improve lithography properties as typified by pattern profile, contrast, mask error factor (MEF), depth of focus (DOF), critical dimension uniformity (CDU), and line width roughness (LWR), but also to minimize defects on the resist pattern after development.

As the pattern feature size is reduced, LWR becomes more noticeable. It is pointed out that LWR is affected by the segregation and agglomeration of a base polymer and an acid generator and acid diffusion. There is a propensity that LWR is degraded as the resist film becomes thinner. The degradation of LWR caused by resist film thinning to comply with further miniaturization becomes a serious problem.

For the EUV resist composition, it is necessary to achieve a high sensitivity, high resolution and low LWR at the same time. As the acid diffusion distance is shortened, the outcome is a smaller LWR, but a lower sensitivity. For example, when the PEB temperature is lowered, LWR becomes smaller, but sensitivity becomes lower. When the amount of an acid diffusion inhibitor or quencher added is increased, LWR becomes smaller, but sensitivity becomes lower. It is necessary to overcome the tradeoff relationship between sensitivity and LWR.

Studies have been made on various additives in order to overcome the tradeoff relationship between sensitivity and LWR. Means for enhancing sensitivity include the structural optimization of photoacid generators and acid diffusion inhibitors such as amines and weak acid onium salts and the addition of acid amplifiers. Additives having a shape-correcting effect are also investigated. Patent Documents 1 to 3 disclose acids and amines of surface segregation type and acid diffusion inhibitors of weak acid onium salt type as shown below.

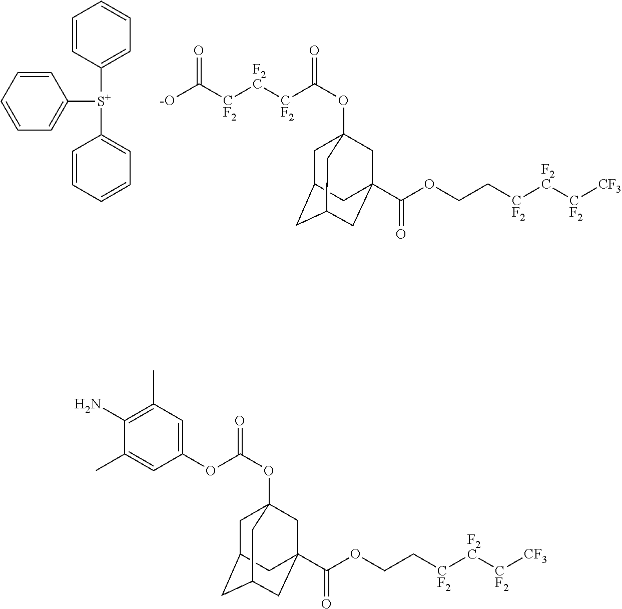

Yet a resist composition capable of meeting LWR and CDU as well as sensitivity has not been developed.

### CITATION LIST

Patent Document 1: JP-A 2019-218340
Patent Document 2: JP-A 2019-026637
Patent Document 3: JP-A 2019-034931

### DISCLOSURE OF INVENTION

While resist patterns of high resolution are recently required, resist compositions comprising conventional acid diffusion inhibitors do not always meet lithography properties such as CDU and LWR.

An object of the invention is to provide a resist composition which when processed by photolithography using high-energy radiation such as KrF or ArF excimer laser, EB or EUV, is improved in lithography properties such as CDU and LWR without a loss of sensitivity. Another object is to provide an acid diffusion inhibitor used in the resist composition and a pattern forming process using the resist composition.

The inventors have found that a resist composition comprising an onium salt having a specific structure as an acid diffusion inhibitor exhibits excellent lithography properties such as CDU and LWR and is suited for precise micropatterning.

In one aspect, the invention provides a salt compound having the formula (1) or (2).

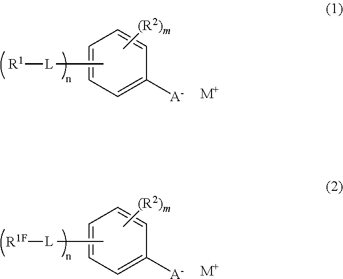

Herein n is an integer of 1 to 5, m is an integer of 0 to 4,

L is a single bond, ether bond or ester bond, L may be the same or different when n is 2 or more,

R¹ is a C₆-C₁₈ alkyl group in which some constituent —CH₂— may be replaced by an ether bond or carbonyl moiety, R¹ has at least one straight structure of 6 or more carbon atoms, R¹ may be the same or different when n is 2 or more, the alkyl group may contain a cyclic structure selected from cyclopentane, cyclohexane, adamantane, norbornane and benzene rings as a partial structure at its end or in its carbon-carbon bond,

R^{1F} is a C₄-C₁₈ fluorinated alkyl group in which some constituent —CH₂— may be replaced by an ether bond or carbonyl moiety, R^{1F} has at least two groups selected from —CF₂— and —CF₃, the fluorinated alkyl group may contain a cyclic structure selected from cyclopentane, cyclohexane, adamantane, norbornane and benzene rings as a partial structure at its end or in its carbon-carbon bond,

R² is halogen, hydroxy, or a C₁-C₁₀ hydrocarbyl group in which some hydrogen may be substituted by halogen and some constituent —CH₂— may be replaced by an ether bond or carbonyl moiety,

M⁺ is a sulfonium or iodonium cation,

A⁻ is an anion having any one of the formulae (A1) to (A4), with the proviso that the partial structure R¹-L- or R^{1F}-L- in formula (1) or (2) is bonded to the benzene ring via —CH₂— or —O— when A⁻ is an anion having formula (A2),

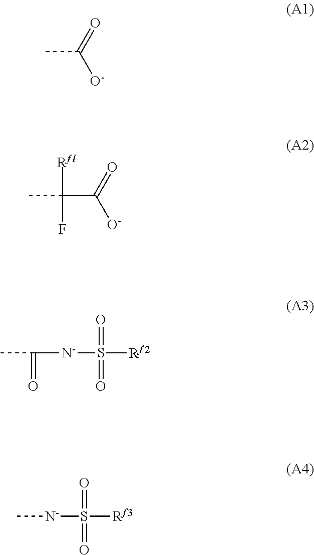

wherein R^{f1} is hydrogen or fluorine, R^{f2} and R^{f3} are each independently methyl, phenyl, tolyl or a C₁-C₄ perfluoroalkyl group, the broken line designates a valence bond.

Preferably, A⁻ is an anion having formula (A1) or (A2). Also preferably, m is 1 or more and at least one R² is iodine.

Preferably, M⁺ is a cation having any one of the formulae (M-1) to (M-3):

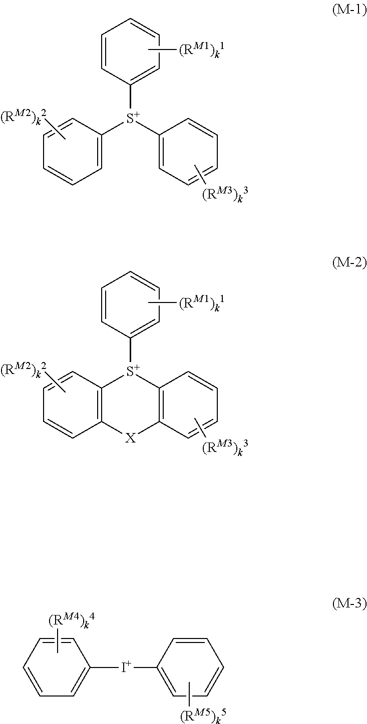

wherein R^{M1}, R^{M2}, R^{M3}, R^{M4} and R^{M5} are each independently hydroxy, halogen, or a C₁-C₁₅ hydrocarbyl group in which some or all of the hydrogen atoms may be substituted by a heteroatom-containing moiety and some constituent —CH₂— may be replaced by —O—, —C(═O)—, —S—, —S(═O)—, —S(═O)₂— or —N(H)—,

k¹, k², k³, k⁴ and k⁵ are each independently an integer of 0 to 5, when k¹ is 2 or more, R^{M1} may be the same or different and two R^{M1} may bond together to form a ring with the carbon atoms on the benzene ring to which they are attached, when k² is 2 or more, R^{M2} may be the same or different and two R^{M2} may bond together to form a ring with the carbon atoms on the benzene ring to which they are attached, when k³ is 2 or more, R^{M3} may be the same or different and two R^{M3} may bond together to form a ring with the carbon atoms on the benzene ring to which they are attached, when k⁴ is 2 or more, R^{M4} may be the same or different and two R^{M4} may bond together to form a ring with the carbon atoms on the benzene ring to which they are attached, when k⁵ is 2 or more, R^{M5} may be the same or different and two R^{M5} may bond together to form a ring with the carbon atoms on the benzene ring to which they are attached, and

X is a single bond, —CH₂—, —O—, —C(═O)—, —S—, —S(═O)—, —S(═O)₂— or —N(H)—.

In a preferred embodiment, the salt compound consists of an anion having the formula (1-I) or (2-I) and a cation having formula (M-1) or (M-2).

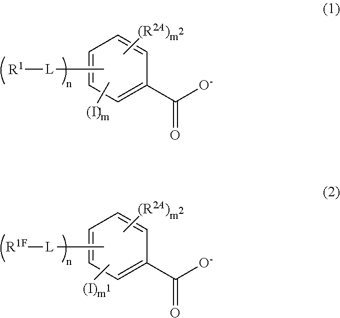

Herein L, R¹, R^{1F} and n are as defined above, R^{2A} is halogen exclusive of iodine, hydroxy, or a C₁-C₁₀ hydrocarbyl group in which some hydrogen may be substituted by halogen and some constituent —CH₂— may be replaced by an ether bond or carbonyl moiety, m¹ is an integer of 1 to 4, m² is an integer of 0 to 3, and n+m¹+m² is from 2 to 5.

In another aspect, the invention provides an acid diffusion inhibitor comprising the salt compound defined above.

In a further aspect, the invention provides:

a resist composition comprising (A) a base polymer adapted to change its solubility in a developer under the action of an acid, (B) a photoacid generator, (C) the acid diffusion inhibitor defined above, and (D) an organic solvent; or

a resist composition comprising (A′) a base polymer adapted to change its solubility in a developer under the action of an acid, the base polymer comprising a photoacid generating site having a function of generating an acid upon exposure to radiation, (C) the acid diffusion inhibitor defined above, and (D) an organic solvent.

In a preferred embodiment, the base polymer is a polymer comprising repeat units having the formula (a) or repeat units having the formula (b).

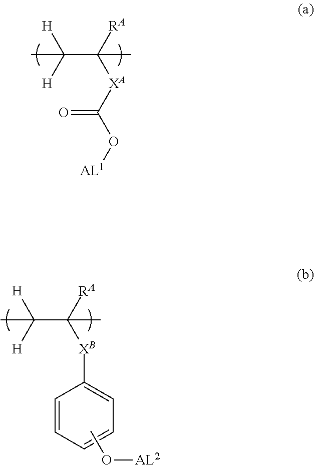

Herein R^{A} is hydrogen or methyl. X^{A} is a single bond, phenylene, naphthylene, or *—C(═O)—O—X^{A1}—, X^{A1} is a C₁-C₁₅ hydrocarbylene group which may contain at least one moiety selected from hydroxy, ether bond, ester bond, and lactone ring, * designates a point of attachment to the carbon atom in the backbone. X^{B} is a single bond or ester bond. AL¹ and AL² are each independently an acid labile group.

In a preferred embodiment, the acid labile group has the formula (L1):

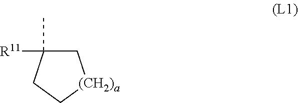

wherein R¹¹ is a C₁-C₇ hydrocarbyl group in which some constituent —CH₂— may be replaced by —O—, a is 1 or 2, and the broken line designates a valence bond.

In a preferred embodiment, the base polymer further comprises repeat units having the formula (c).

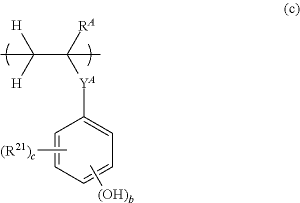

Herein R^{A} is hydrogen or methyl, Y^{A} is a single bond or ester bond, R²¹ is fluorine, iodine or a C₁-C₁₀ hydrocarbyl group in which some constituent —CH₂— may be replaced by —O— or —C(═O)—, b is an integer of 1 to 5, c is an integer of 0 to 4, and b+c is from 1 to 5.

In a preferred embodiment, the base polymer further comprises repeat units of at least one type selected from the formulae (d1) to (d4):

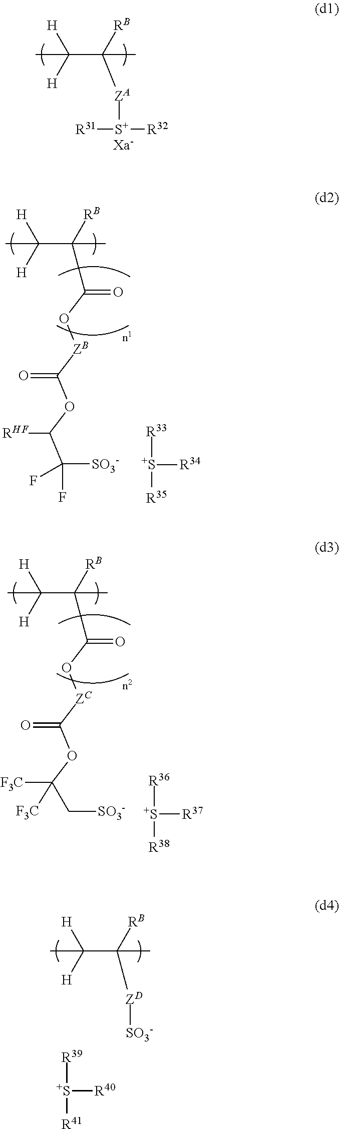

Herein R^{B} is hydrogen, fluorine, methyl or trifluoromethyl. Z^{A} is a single bond, phenylene group, —O—Z^{A1}—, —C(═O)—O—Z^{A1}— or —C(═O)—N(H)—Z^{A1}—, wherein Z^{A}1 is a C₁-C₂₀ hydrocarbylene group which may contain a heteroatom. Z^{B} and Z^{C} are each independently a single bond or a C₁-C₂₀ hydrocarbylene group which may contain a heteroatom. Z^{D} is a single bond, methylene, ethylene, phenylene, fluorinated phenylene, —O—Z^{D1}—, —C(═O)—OZ^{D1}— or —C(═O)—N(H)—Z^{D1}—, wherein Z^{D1} is an optionally substituted phenylene group. R³¹ to R⁴¹ are each independently a C₁-C₂₀ hydrocarbyl group which may contain a heteroatom, any two or more of Z^{A}, R³¹ and R³² may bond together to form a ring with the sulfur atom to which they are attached, any two of R³³, R³⁴ and R³⁵, any two or more of R³⁶, R³⁷ and R³⁸, and any two or more of R³⁹, R⁴⁰ and R⁴¹ may bond together to form a ring with the sulfur atom to which they are attached. R^{HF} is hydrogen or trifluoromethyl. The subscript n¹ is 0 or 1, n¹ is 0 when Z^{B} is a single bond, n² is 0 or 1, n² is 0 when Z^{C} is a single bond. Xa⁻ is a non-nucleophilic counter ion.

In a still further aspect, the invention provides a pattern forming process comprising the steps of applying the resist composition defined above onto a substrate to form a resist film thereon, exposing a selected region of the resist film to KrF excimer laser, ArF excimer laser, EB or EUV, and developing the exposed resist film in a developer.

In a preferred embodiment, the developing step uses an alkaline aqueous solution as the developer, thereby forming a positive pattern in which an exposed region of the resist film is dissolved away and an unexposed region of the resist film is not dissolved.

In another preferred embodiment, the developing step uses an organic solvent as the developer, thereby forming a negative pattern in which an unexposed region of the resist film is dissolved away and an exposed region of the resist film is not dissolved.

The organic solvent is preferably selected from among 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate.

### Advantageous Effects of Invention

When the inventive resist composition comprising the salt compound as an acid diffusion inhibitor is processed by lithography, a resist pattern having improved lithography properties such as CDU, LWR and DOF can be formed.

### BRIEF DESCRIPTION OF DRAWINGS

FIG. 1 is a diagram showing the ¹H-NMR spectrum of sulfonium salt Q-1 obtained in Example 1-1.

FIG. 2 is a diagram showing the ¹H-NMR spectrum of sulfonium salt Q-2 obtained in Example 1-2.

FIG. 3 is a diagram showing the ¹H-NMR spectrum of sulfonium salt Q-3 obtained in Example 1-3.

### DESCRIPTION OF THE PREFERRED EMBODIMENTS

The singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. The terms “group” and “moiety” are interchangeable. In chemical formulae, the broken line denotes a valence bond; Me stands for methyl, tBu for tert-butyl, Ac for acetyl, and Ph for phenyl. It is understood that for some structures represented by chemical formulae, there can exist enantiomers and diastereomers because of the presence of asymmetric carbon atoms. In such a case, a single formula collectively represents all such isomers. The isomers may be used alone or in admixture. The high-energy radiation encompasses UV, deep UV, EB, EUV, X-ray, γ-ray and synchrotron radiation.

The abbreviations have the following meaning.
EB: electron beam
EUV: extreme ultraviolet
GPC: gel permeation chromatography
Mw: weight average molecular weight
Mw/Mn: molecular weight dispersity
PAG: photoacid generator
PEB: post-exposure bake
LWR: line width roughness
CDU: critical dimension uniformity
DOF: depth of focus
Salt

The invention provides a salt compound having the formula (1) or (2).

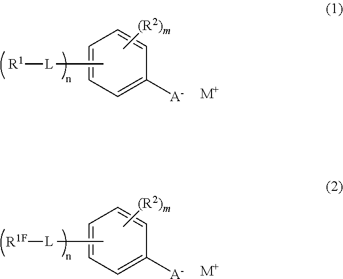

In formulae (1) and (2), n is an integer of 1 to 5, preferably 1 or 2, m is an integer of 0 to 4, preferably 0, 1 or 2.

In formulae (1) and (2), L is a single bond, ether bond or ester bond, preferably an ether bond. L may be the same or different when n is 2 or more.

In formula (1), R¹ is a C₆-C₁₈ alkyl group in which some constituent —CH₂— may be replaced by an ether bond or carbonyl moiety, with the proviso that R¹ has at least one straight structure of 6 or more carbon atoms. R¹ may be the same or different when n is 2 or more.

Examples of the C-Cis alkyl group R¹ include 1-hexyl, 1-heptyl, 1-octyl, 1-nonyl, 1-decyl, 1-undecyl, 1-dodecyl, 1-tridecyl, 1-tetradecyl, 1-hexadecyl, 1-octadecyl, octan-2-yl, decan-2-yl, decan-4-yl, octadecan-8-yl, 7,7-dimethyloctyl, 7,7-diethylnonyl, and 4-butyldodecyl. The alkyl group may contain a cyclic structure such as cyclopentane, cyclohexane, adamantane, norbornane or benzene ring as a partial structure at its end or in its chain structure. In the alkyl group, some constituent —CH₂— may be replaced by an ether bond or carbonyl moiety, and as a result of replacement, an ester bond or lactone ring may be formed. R¹ is preferably a straight alkyl group or straight glyme chain.

In formula (2), RF is a C₄-C₁₈ fluorinated alkyl group in which some constituent —CH₂— may be replaced by an ether bond or carbonyl moiety. R^{1F} has at least two groups selected from —CF₂— and —CF₃.

Examples of the C₄-C₁₈ fluorinated alkyl group R^{1F} include 1-butyl, 1-pentyl, 1-hexyl, 1-heptyl, 1-octyl, 1-nonyl, 1-decyl, 1-undecyl, 1-dodecyl, 1-tridecyl, 1-tetradecyl, 1-hexadecyl, 1-octadecyl, octan-2-yl, decan-2-yl, decan-4-yl, octadecan-8-yl, 7,7-dimethyloctyl, 7,7-diethylnonyl, and 4-butyldodecyl, in which some or all hydrogen atoms are substituted by fluorine. The fluorinated alkyl group may contain a cyclic structure such as cyclopentane, cyclohexane, adamantane, norbornane or benzene ring as a partial structure at its end or in its chain structure. In the fluorinated alkyl group, some constituent —CH₂— may be replaced by an ether bond or carbonyl moiety, and as a result of replacement, an ester bond or lactone ring may be formed.

Preferred examples of R^{1F} are shown below.

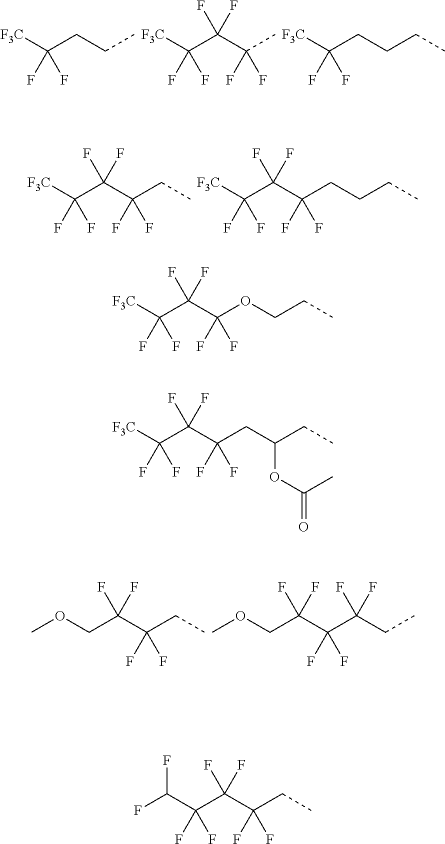

In formulae (1) and (2), R² is halogen, hydroxy, or a C₁-C₁₀ hydrocarbyl group in which some hydrogen may be substituted by halogen and some constituent —CH₂— may be replaced by an ether bond or carbonyl moiety. Examples of R² include fluorine, iodine, hydroxy, methyl, trifluoromethyl, methoxy, trifluoromethoxy, ethyl, n-propyl, isopropyl, n-butyl, sec-butyl, tert-butyl, cyclohexyl, phenyl, adamantyl, 2,2,2-trifluoroethoxy, 2-methoxyethoxy, 2-hydroxy-2-trifluoromethyl-3,3,3-trifluoropropoxy, acetyl, and acetoxy. Inter alia, hydroxy, fluorine, iodine, trifluoromethyl, methyl and tert-butyl are preferred.

In formulae (1) and (2), A⁻ is an anion having any one of the following formulae (A1) to (A4). The partial structure R¹-L- or R^{1F}-L- in formula (1) or (2) is bonded to the benzene ring via —CH₂— or —O— when A⁻ is an anion having formula (A2).

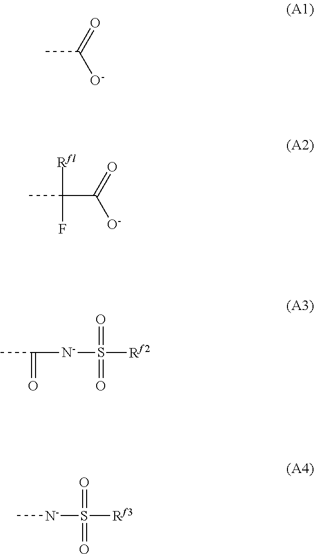

In formulae (A1) to (A4), R^{f1} is hydrogen or fluorine, preferably fluorine. R^{f2} and R^{f3} are each independently methyl, phenyl, tolyl or a C₁-C₄ perfluoroalkyl group, preferably perfluoromethyl.

A⁻ is preferably an anion having formula (A1) or (A2), more preferably an anion having formula (A1).

Examples of the anion in the salt having formula (1) are shown below, but not limited thereto.

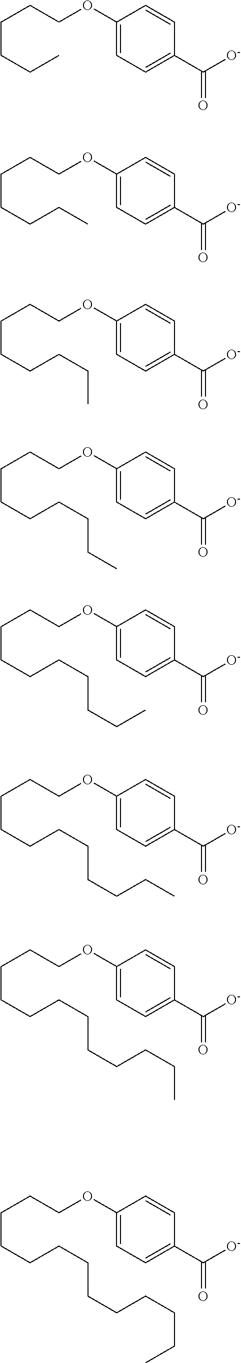

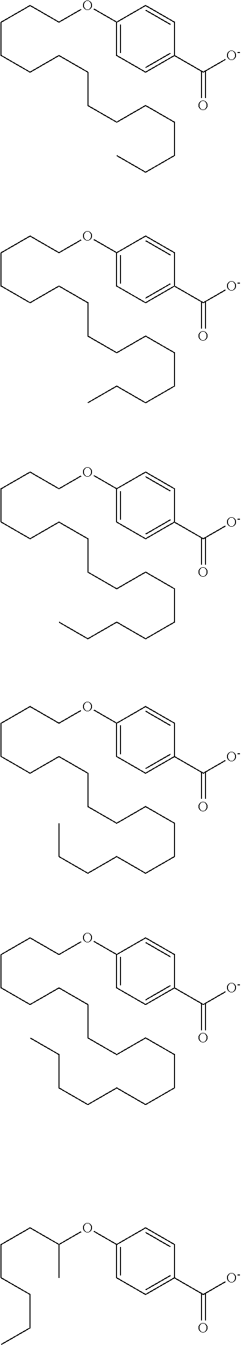 [SMILES: CCCCCCCCCCCCCCOc1ccc(C(=O)[O-])cc1 ; CCCCCCCCCCCCCCCOc1ccc(C(=O)[O-])cc1 ; CCCCCCCCCCCCCCCCOc1ccc(C(=O)[O-])cc1 ; CCCCCCCCCCCCCCCCCOc1ccc(C(=O)[O-])cc1 ; CCCCCCCCCCCCCCCCCCOc1ccc(C(=O)[O-])cc1 ; CCCCCCC(C)Oc1ccc(C(=O)[O-])cc1]

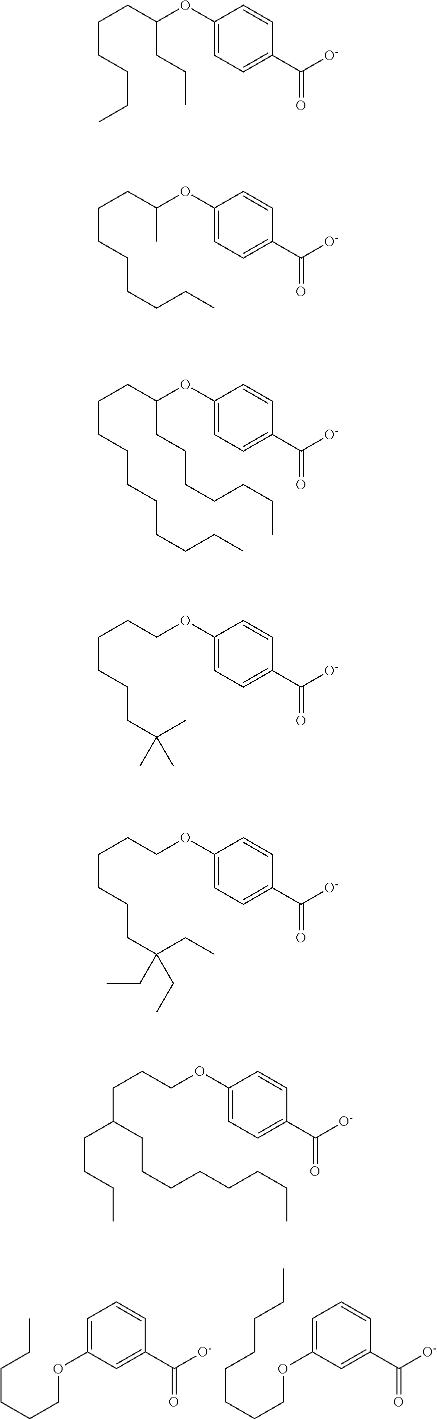

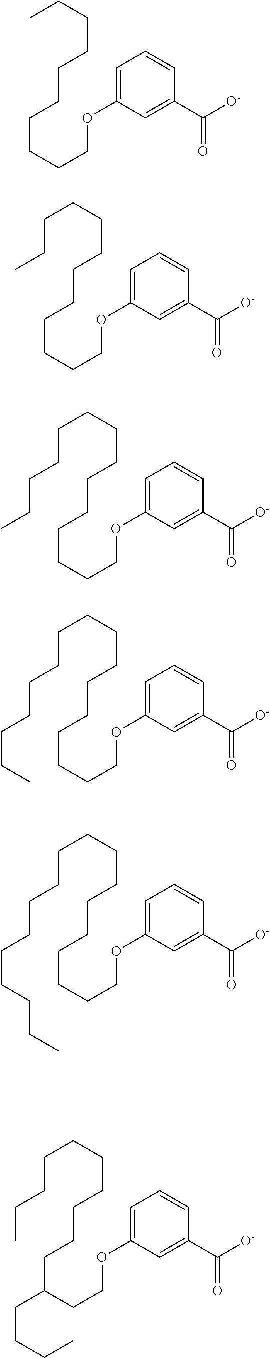 [SMILES: CCCCCCCCCCOc1cccc(C(=O)[O-])c1 ; CCCCCCCCCCCCOc1cccc(C(=O)[O-])c1 ; CCCCCCCCCCCCCCOc1cccc(C(=O)[O-])c1 ; CCCCCCCCCCCCCCCCOc1cccc(C(=O)[O-])c1 ; CCCCCCCCCCCCCCCCCCOc1cccc(C(=O)[O-])c1 ; CCCCCCCCCCC(CCCC)CCOc1cccc(C(=O)[O-])c1]

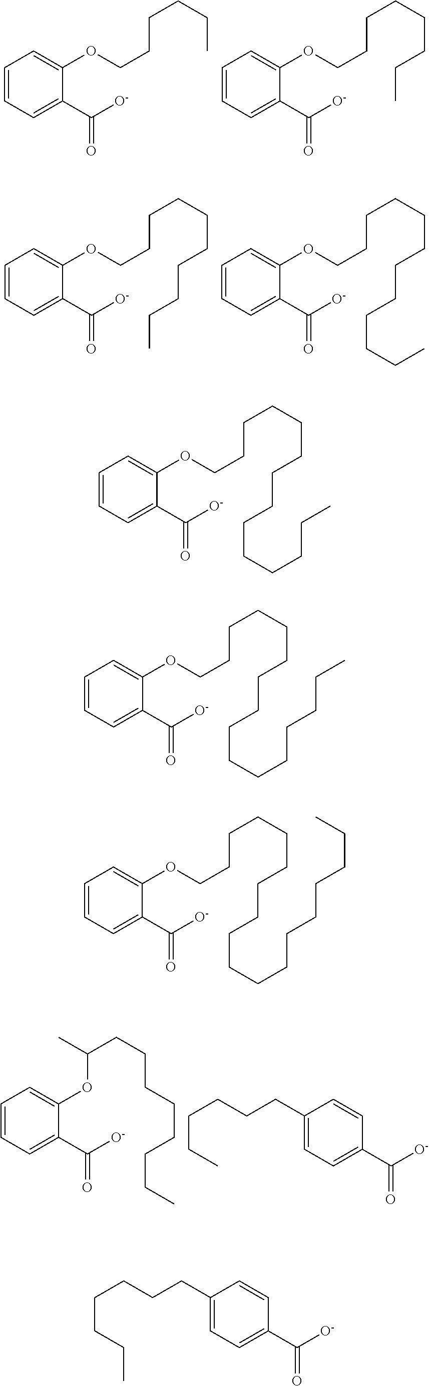

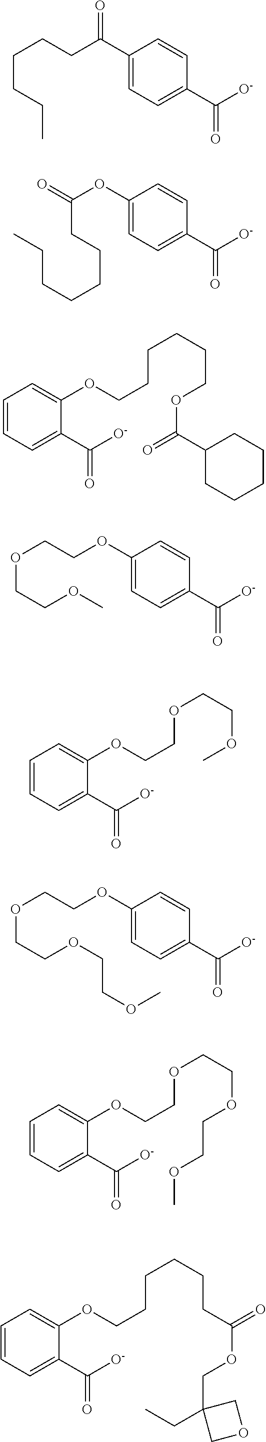

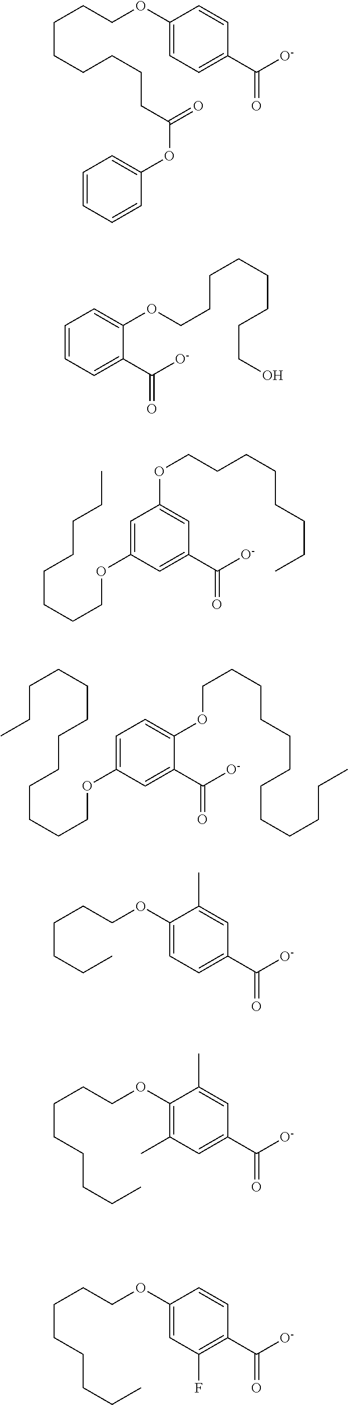

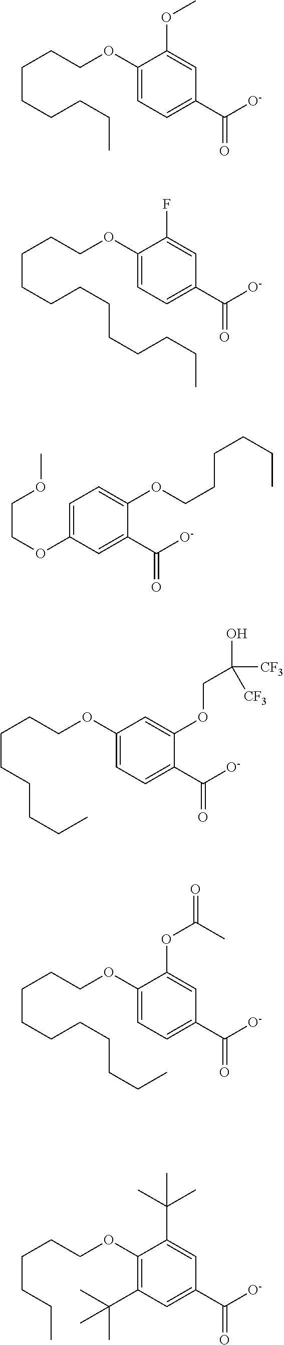 [SMILES: CCCCCCCCOc1ccc(C(=O)[O-])cc1OC ; CCCCCCCCCCCCOc1ccc(C(=O)[O-])cc1F ; CCCCCCOc1ccc(OCCOC)cc1C(=O)[O-] ; CCCCCCCCOc1ccc(C(=O)[O-])c(OCC(O)(C(F)(F)F)C(F)(F)F)c1 ; CCCCCCCCCCOc1ccc(C(=O)[O-])cc1OC(C)=O ; CCCCCCOc1c(C(C)(C)C)cc(C(=O)[O-])cc1C(C)(C)C]

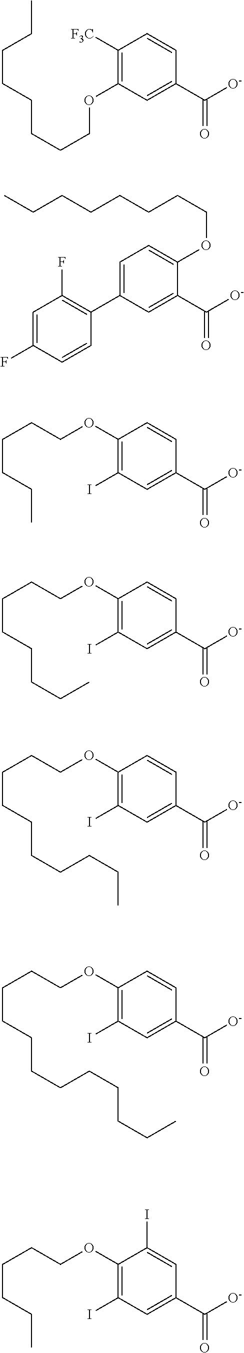

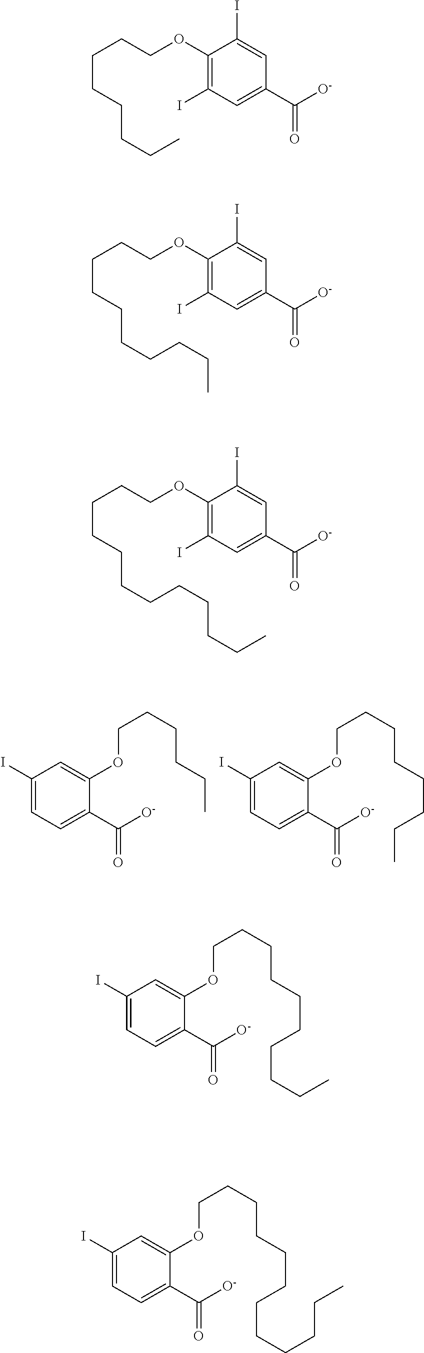

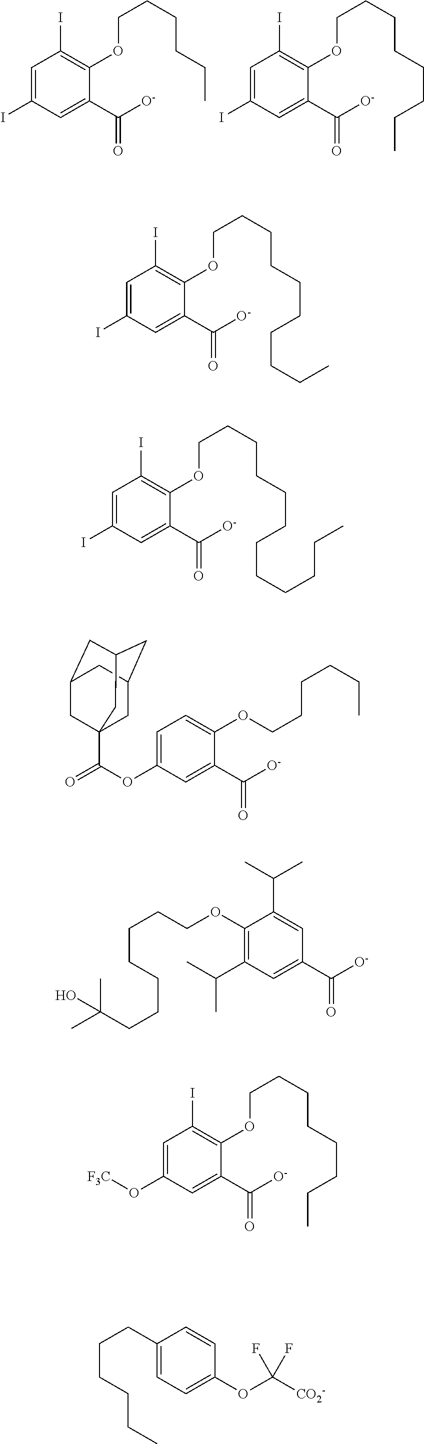

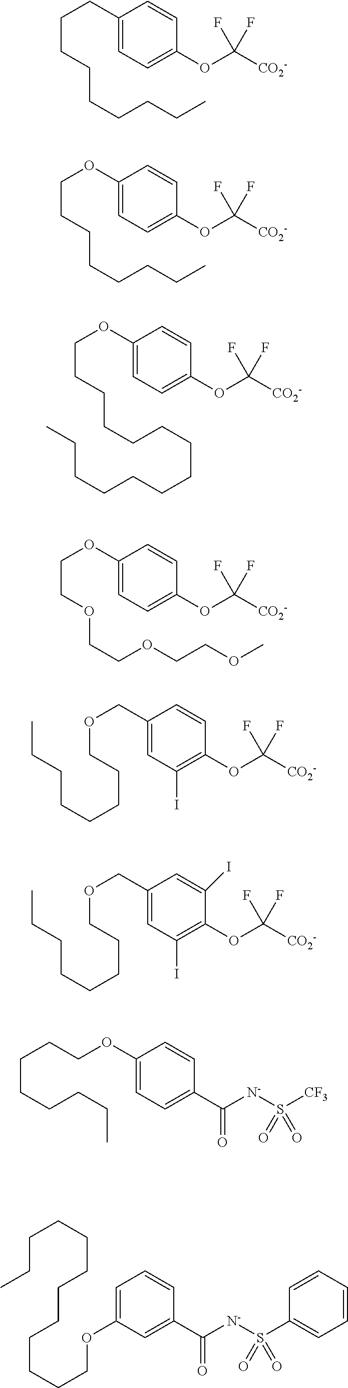

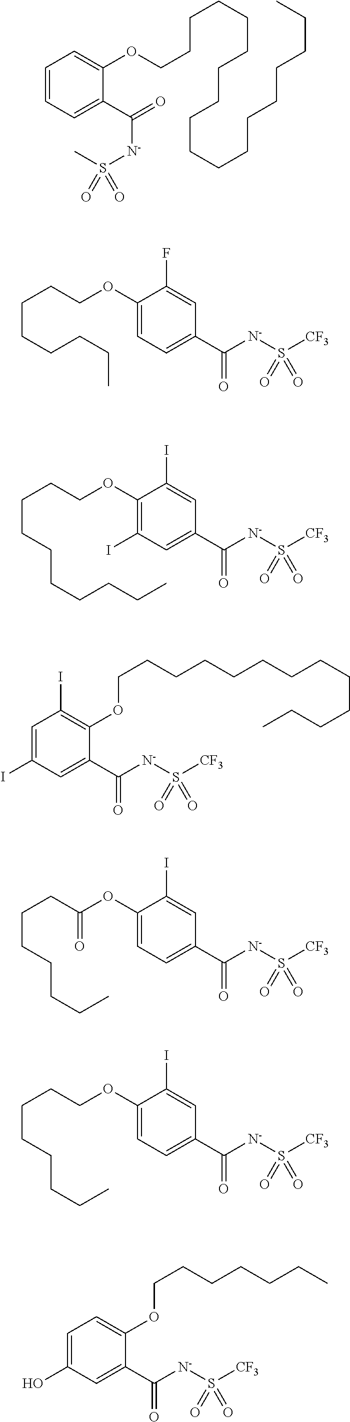

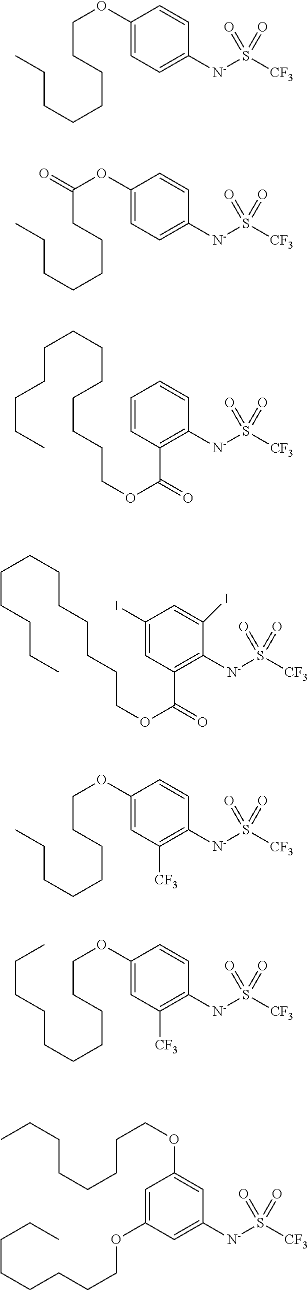

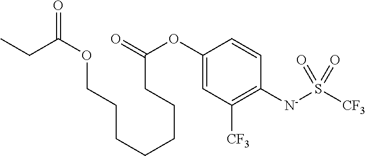 [SMILES: CCC(=O)OCCCCCCCC(=O)Oc1ccc([N-]S(=O)(=O)C(F)(F)F)c(C(F)(F)F)c1]

Examples of the anion in the salt having formula (2) are shown below, but not limited thereto.

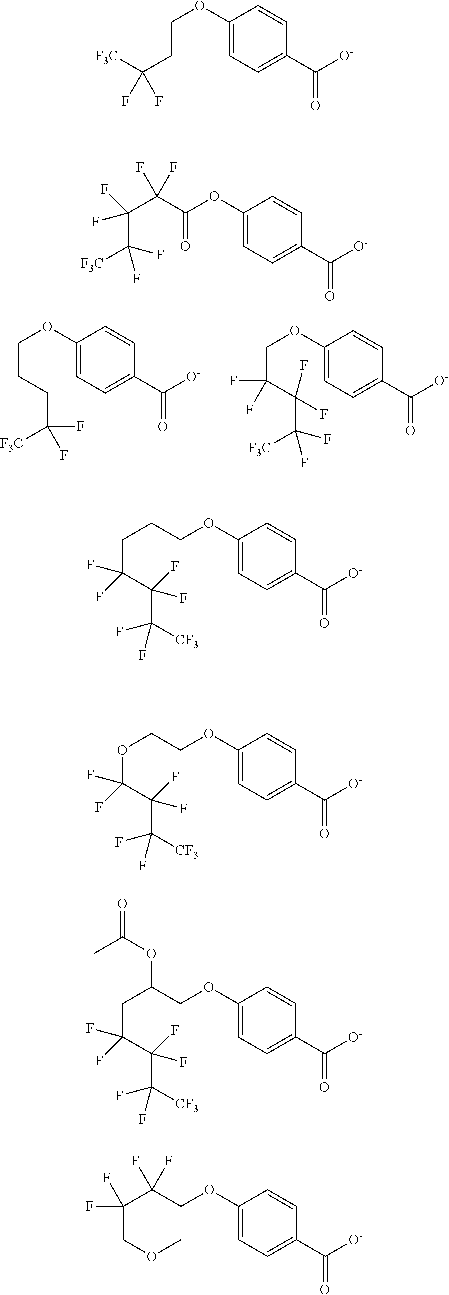

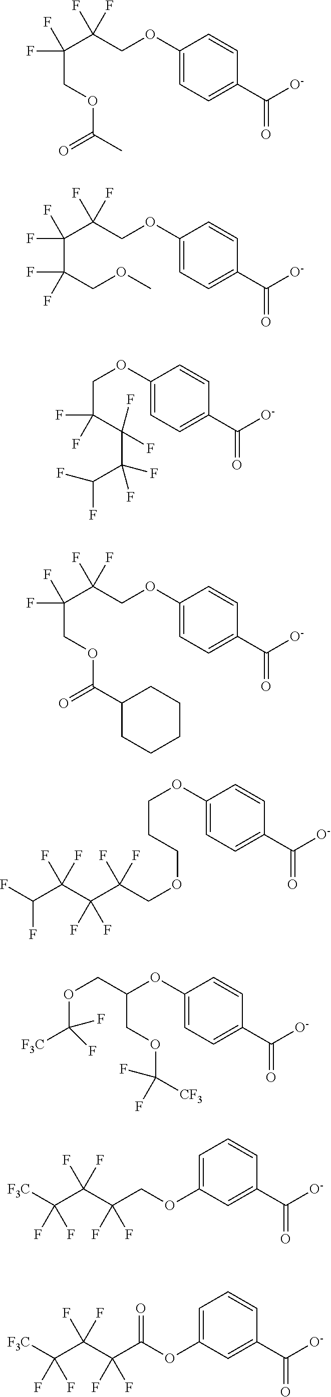

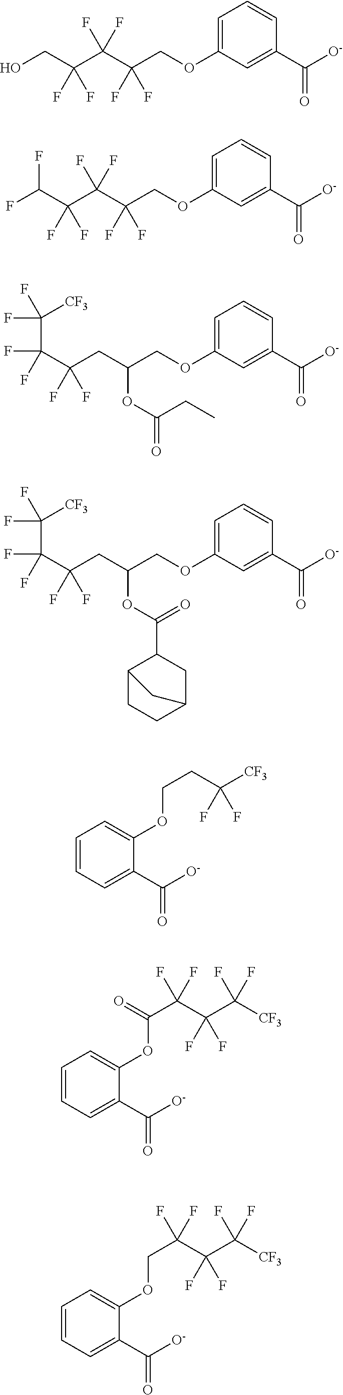

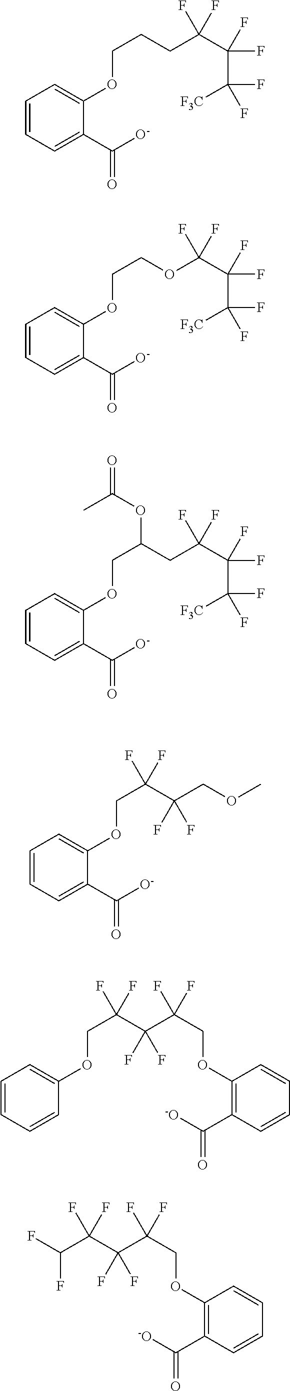

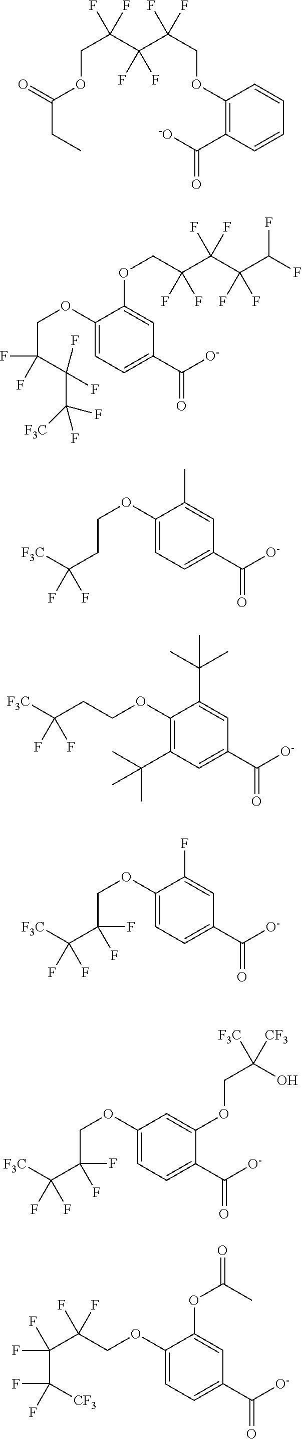

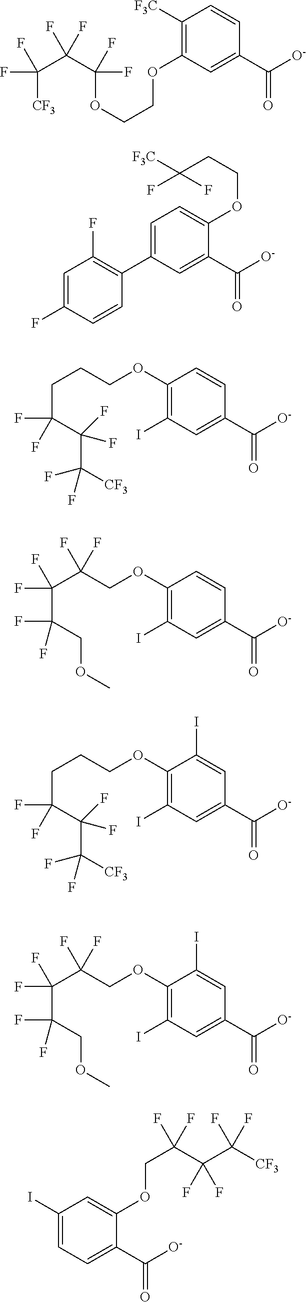

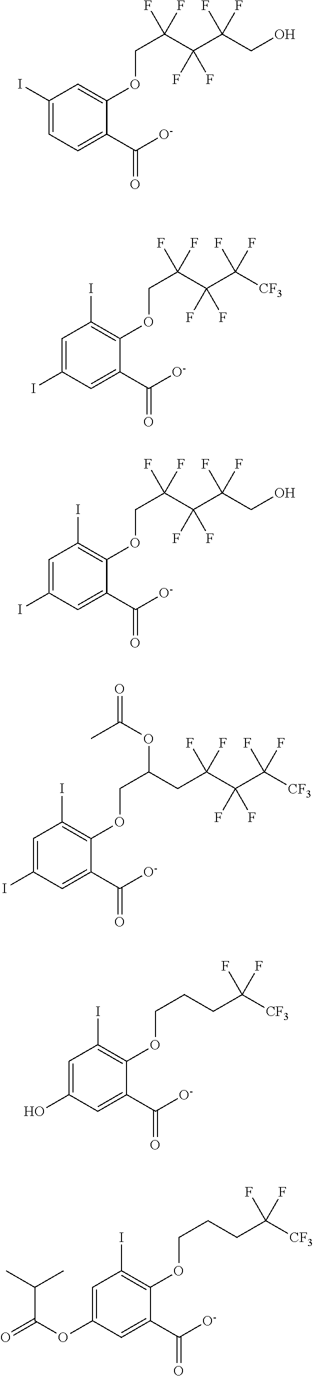 [SMILES: O=C([O-])c1ccc(I)cc1OCC(F)(F)C(F)(F)C(F)(F)CO ; O=C([O-])c1cc(I)cc(I)c1OCC(F)(F)C(F)(F)C(F)(F)C(F)(F)F ; O=C([O-])c1cc(I)cc(I)c1OCC(F)(F)C(F)(F)C(F)(F)CO ; CC(=O)OC(COc1c(I)cc(I)cc1C(=O)[O-])CC(F)(F)C(F)(F)C(F)(F)C(F)(F)F ; O=C([O-])c1cc(O)cc(I)c1OCCCC(F)(F)C(F)(F)F ; CC(C)C(=O)Oc1cc(I)c(OCCCC(F)(F)C(F)(F)F)c(C(=O)[O-])c1]

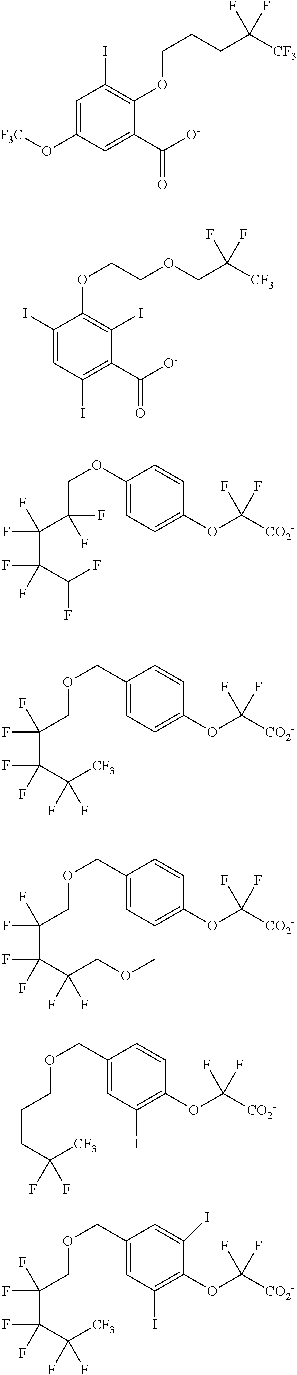

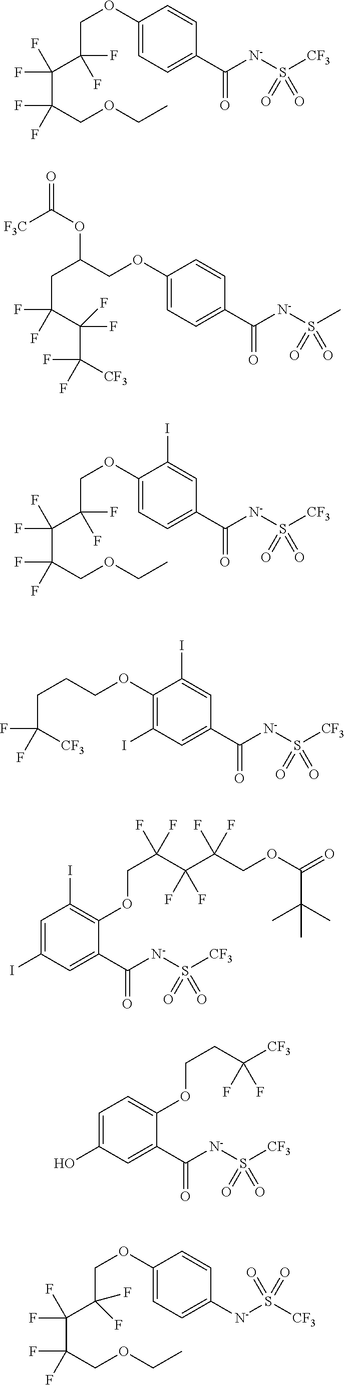

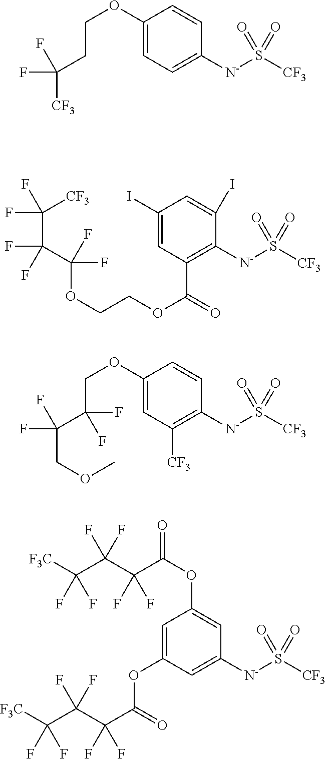 [SMILES: O=S(=O)([N-]c1ccc(OCCC(F)(F)C(F)(F)F)cc1)C(F)(F)F ; O=C(OCCOC(F)(F)C(F)(F)C(F)(F)C(F)(F)F)c1cc(I)cc(I)c1[N-]S(=O)(=O)C(F)(F)F ; COCC(F)(F)C(F)(F)COc1ccc([N-]S(=O)(=O)C(F)(F)F)c(C(F)(F)F)c1 ; O=C(Oc1cc([N-]S(=O)(=O)C(F)(F)F)cc(OC(=O)C(F)(F)C(F)(F)C(F)(F)C(F)(F)F)c1)C(F)(F)C(F)(F)C(F)(F)C(F)(F)F]

In formulae (1) and (2), M⁺ is a sulfonium or iodonium cation. M⁺ is preferably a cation having at least one benzene ring bonded to its center. More preferably, M⁺ is selected from cations having the formulae (M-1) to (M-3).

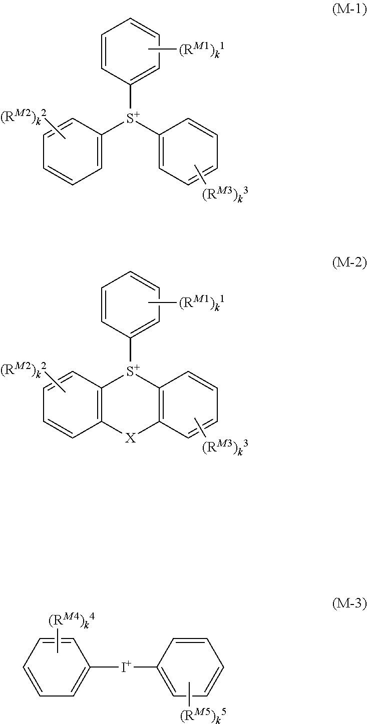

In formulae (M-1) to (M-3), R^{M1}, R^{M2}, R^{M3}, R^{M4}, and R^{M5} are each independently hydroxy, halogen, or a C₁-C₁₅ hydrocarbyl group.

Suitable halogen atoms include fluorine, chlorine, bromine and iodine. The C₁-C₁₅ hydrocarbyl group may be saturated or unsaturated and straight, branched or cyclic. Examples include alkyl groups such as methyl, ethyl, n-propyl, isopropyl, n-butyl, sec-butyl, tert-butyl, n-pentyl, n-hexyl, n-octyl, n-nonyl, and n-decyl; cyclic saturated hydrocarbyl groups such as cyclopentyl, cyclohexyl, and adamantyl; aromatic hydrocarbyl groups such as phenyl, and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by —O—, —C(═O)—, —S—, —S(═O)—, —S(═O)₂— or —N(H)—. That is, the hydrocarbyl group may contain a hydroxy moiety, cyano moiety, carbonyl moiety, ether bond, ester bond, amide bond, thioether bond, sulfonic ester bond, carbonate bond, lactone ring, sultone ring, carboxylic anhydride (—C(═O)—O—C(═O)—) or haloalkyl moiety. Notably the constituent —CH₂— in the hydrocarbyl group may be one bonded to a carbon atom on the benzene ring in formulae (M-1) to (M-3). In this case, R^{M1} to R^{M5} may be hydrocarbyloxy, hydrocarbylcarbonyloxy, hydrocarbylthio, hydrocarbylcarbonyl, hydrocarbylsulfonyl, or hydrocarbylamino.

In formulae (M-1) to (M-3), k¹, k², k³, k⁴ and k⁵ are each independently an integer of 0 to 5. When k¹ is 2 or more, a plurality of R^{M1} may be the same or different, and two R^{M1} may bond together to form a ring with the carbon atoms on the benzene ring to which they are attached. When k² is 2 or more, a plurality of R^{M2} may be the same or different, and two R^{M2} may bond together to form a ring with the carbon atoms on the benzene ring to which they are attached. When k³ is 2 or more, a plurality of R^{M3} may be the same or different, and two R^{M3} may bond together to form a ring with the carbon atoms on the benzene ring to which they are attached. When k⁴ is 2 or more, a plurality of R^{M4} may be the same or different, and two R^{M4} may bond together to form a ring with the carbon atoms on the benzene ring to which they are attached. When k⁵ is 2 or more, a plurality of R^{M5} may be the same or different, and two R^{M5} may bond together to form a ring with the carbon atoms on the benzene ring to which they are attached.

In formula (M-2), X is a single bond, —CH₂—, —O—, —C(═O)—, —S—, —S(═O)—, —S(═O)₂—or —N(H)—.

Examples of the sulfonium cation represented by M⁺ are given below, but not limited thereto.

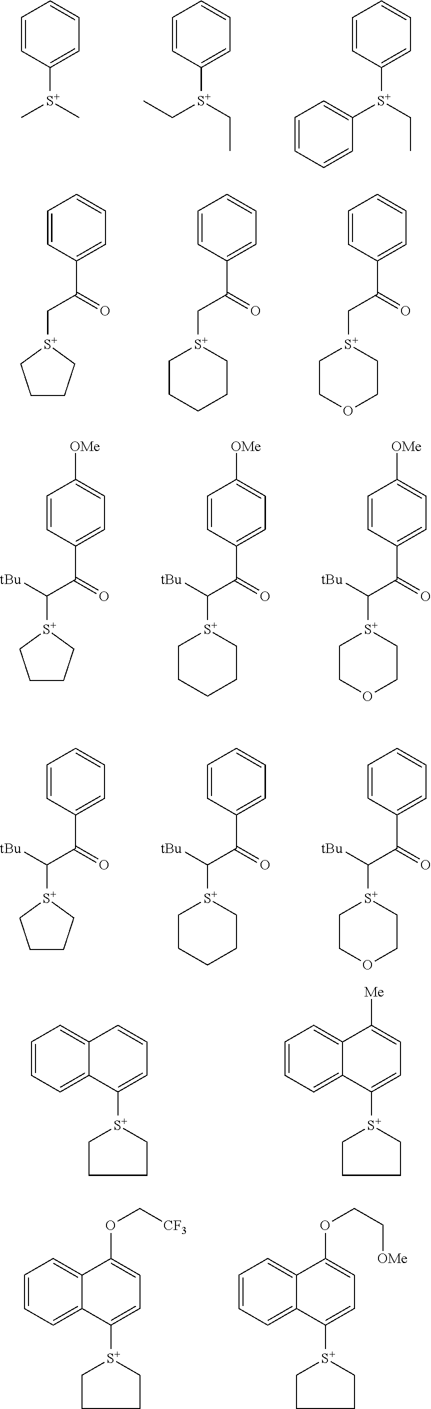

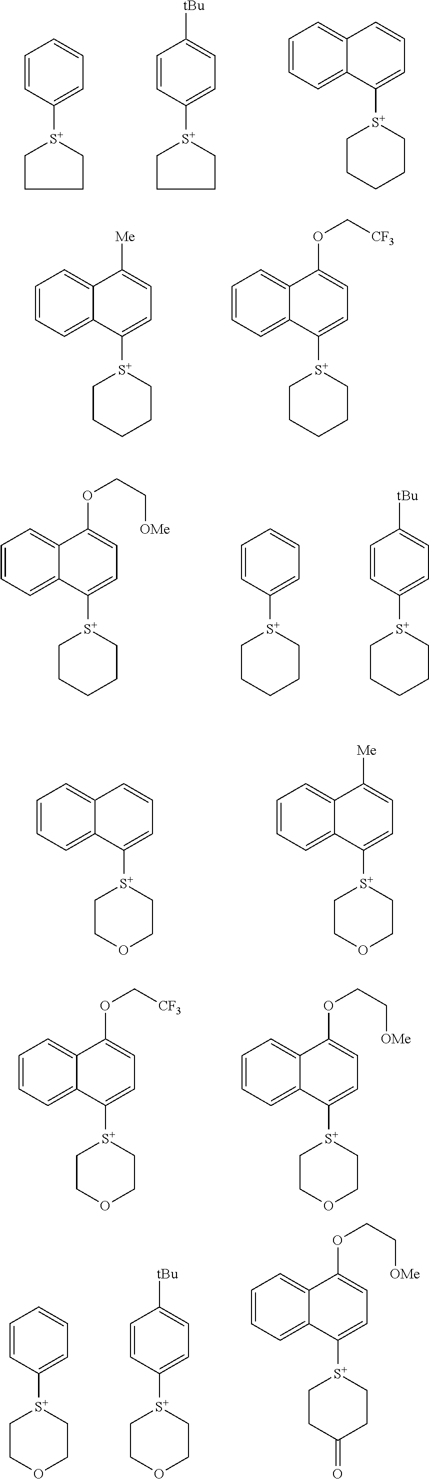

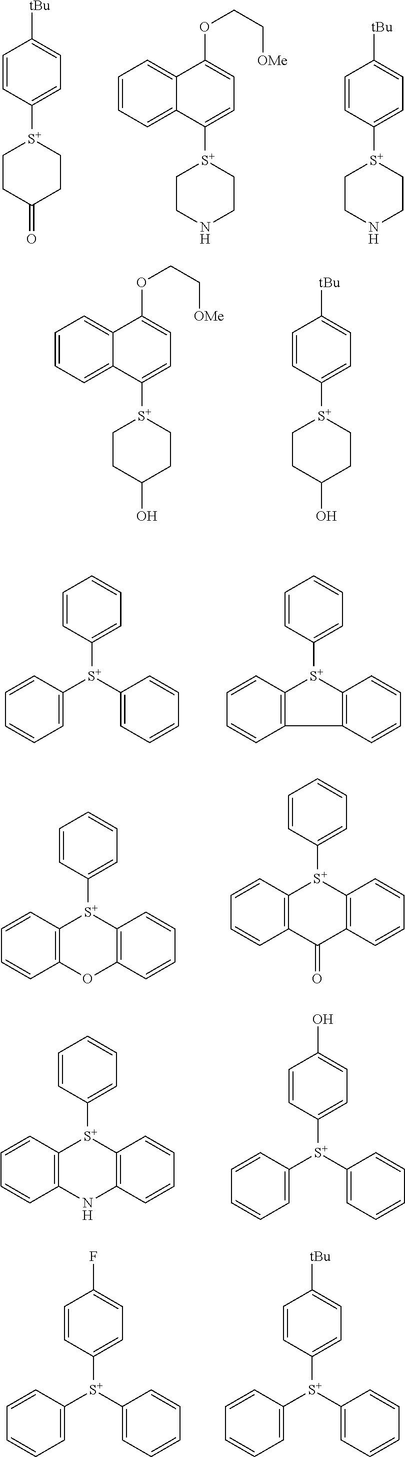

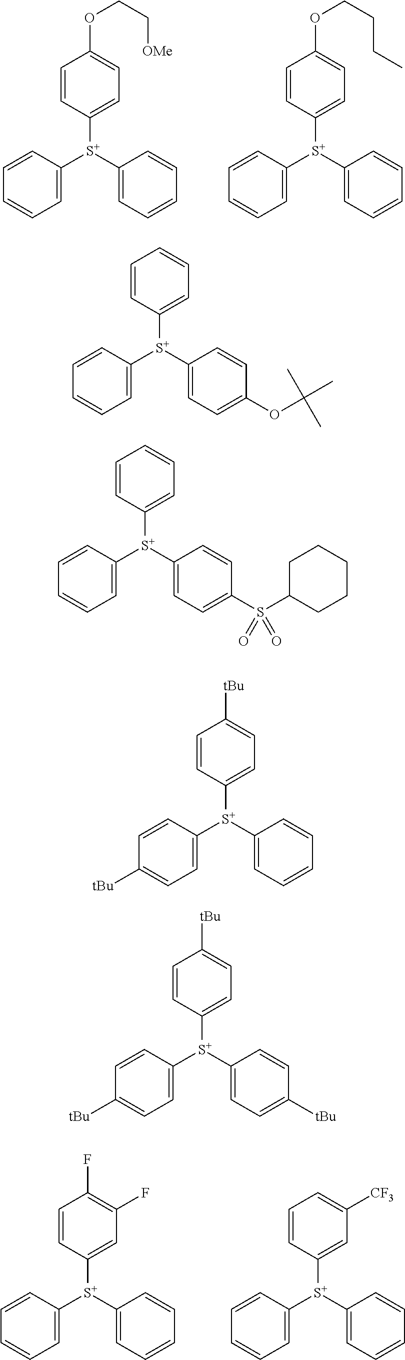

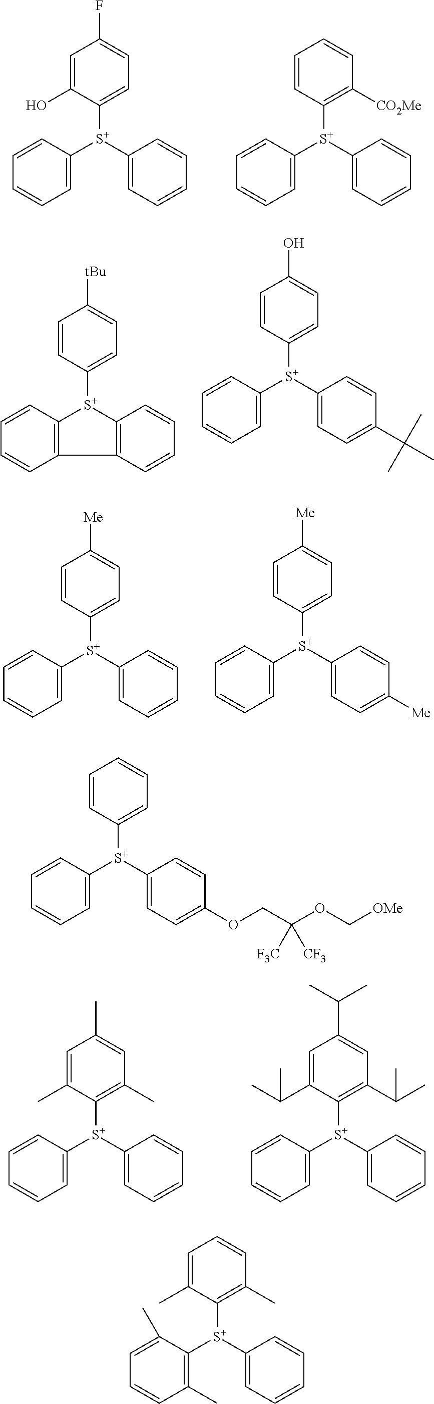

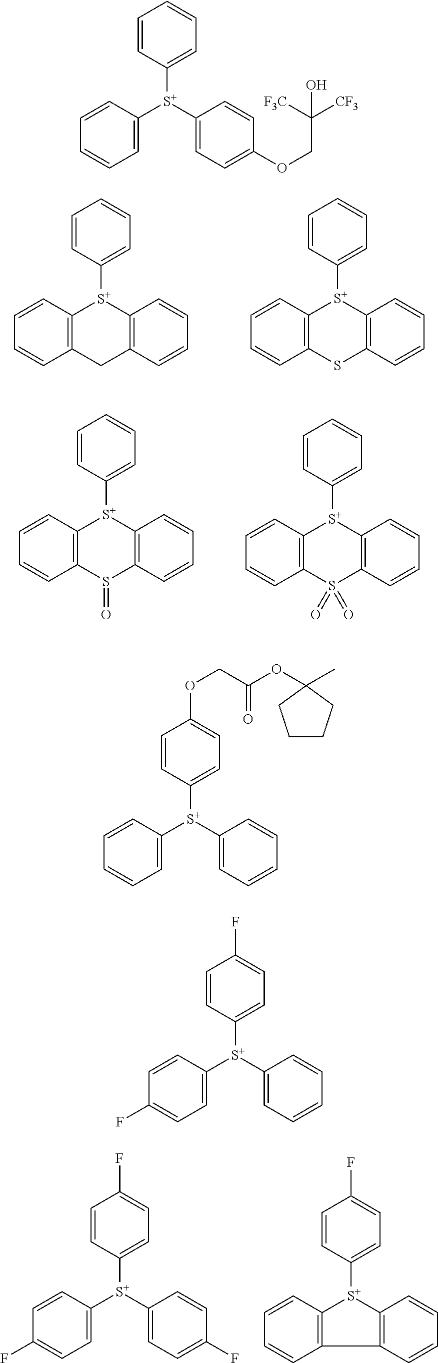

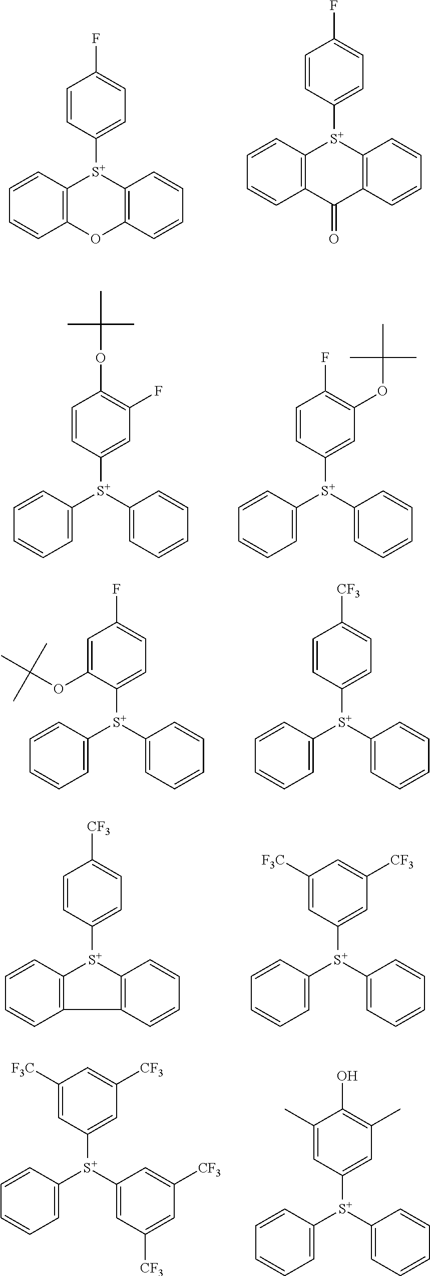

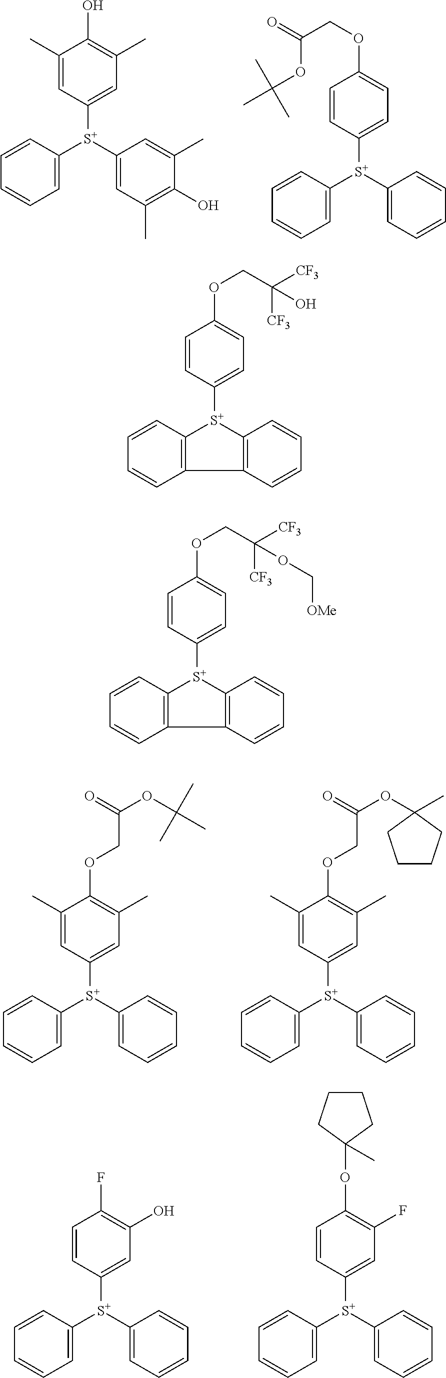

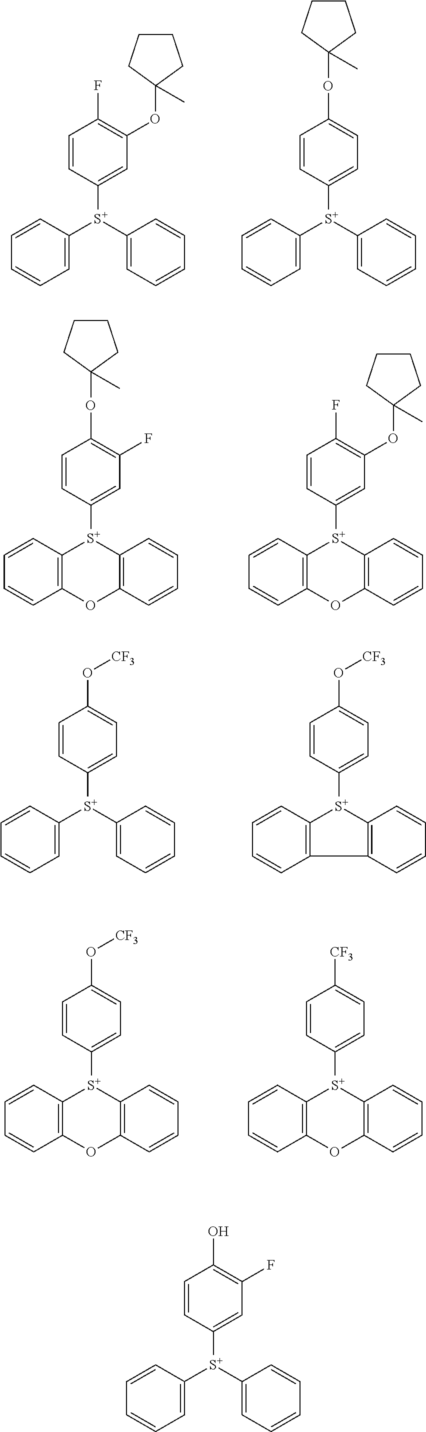

Examples of the iodonium cation represented by M⁺ are given below, but not limited thereto.

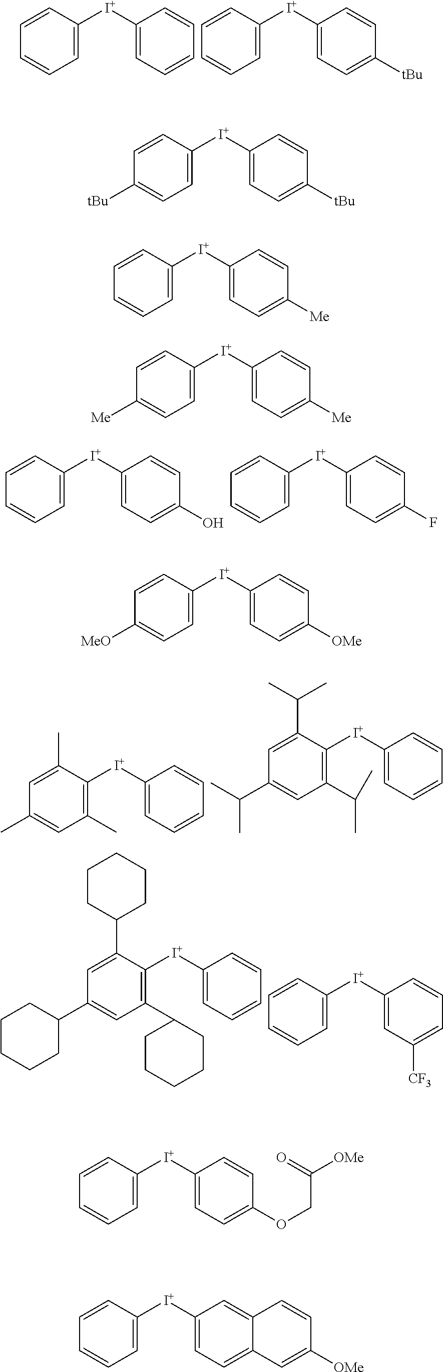

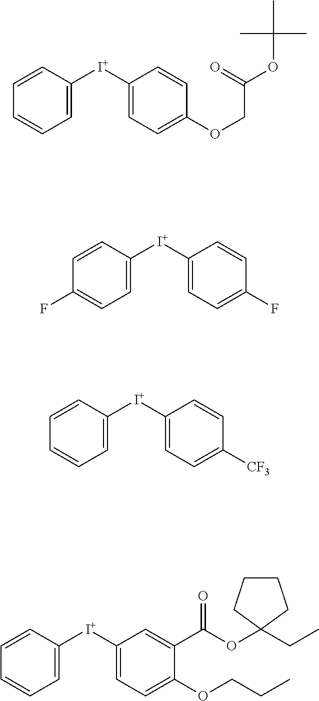 [SMILES: CC(C)(C)OC(=O)COc1ccc([I+]c2ccccc2)cc1 ; Fc1ccc([I+]c2ccc(F)cc2)cc1 ; FC(F)(F)c1ccc([I+]c2ccccc2)cc1 ; CCCOc1ccc([I+]c2ccccc2)cc1C(=O)OC1(CC)CCCC1]

Of the salts having formulae (1) and (2), salts consisting of an anion having formula (1-I) or (2-I) and a cation having formula (M-1) or (M-2) are preferred.

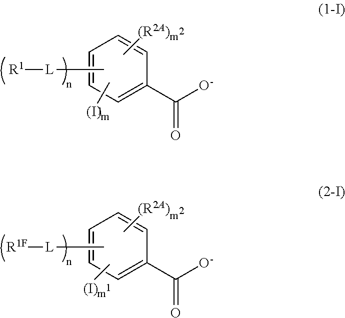

Herein L, R¹, R^{1F} and n are as defined above. R^{2A} is halogen exclusive of iodine, hydroxy, or a C₁-C₁₀ hydrocarbyl group in which some hydrogen may be substituted by halogen and some constituent —CH₂— may be replaced by an ether bond or carbonyl moiety. The subscript m¹ is an integer of 1 to 4, m² is an integer of 0 to 3, and n+m¹+m² is from 2 to 5.

Of the salts having formulae (1) and (2), those salts consisting of an anion and a cation both as shown below are especially preferred.

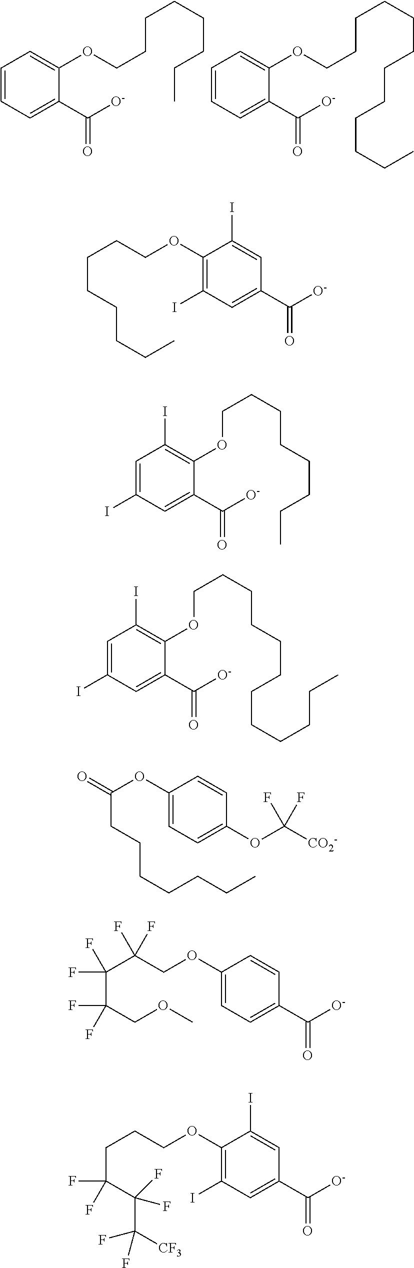

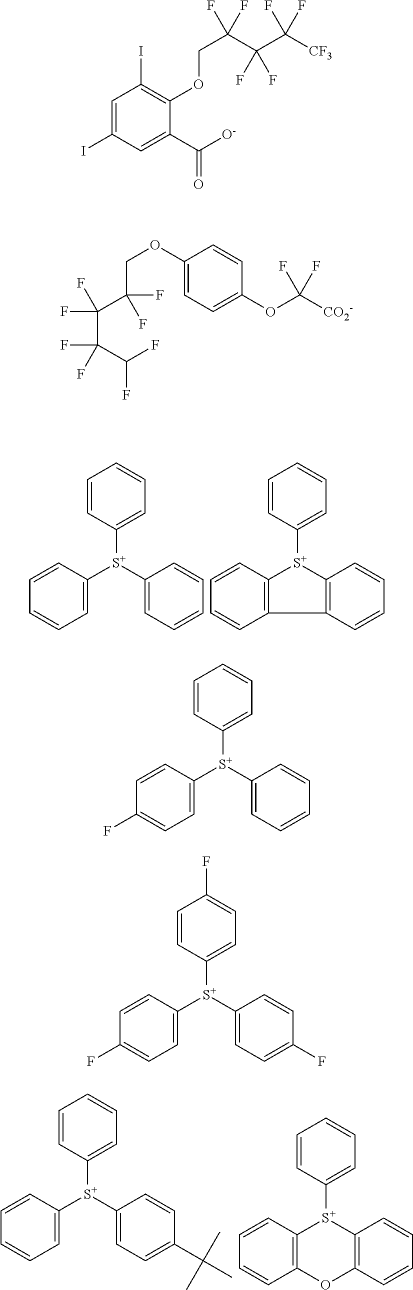

The salt compound may be synthesized, for example, according to the following scheme.

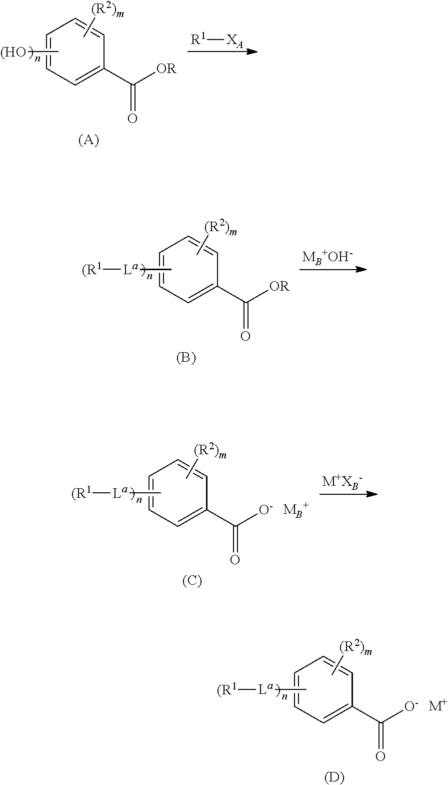

Herein R¹, R², M⁺, m and n are as defined above. R is hydrogen, methyl or 2,2,2-trifluoroethyl. L is an ether bond or ester bond. X_{A} is chlorine, bromine or iodine when L is an ether bond, or —C(═O)—Cl when L is an ester bond. M_{B}⁺ is a monovalent metal ion or tetramethylammonium cation. X_{B}⁻ is an anion.

In the first step, a phenol derivative (A) is reacted with a halide (R¹—X_{A}) under basic conditions for etherification to synthesize an intermediate (B). When an acid chloride is used as the halide (R¹—X_{A}), it may be similarly reacted with the phenol derivative (A) under basic conditions for esterification, for thereby synthesizing the intermediate (B). The etherification reaction may be performed in a polar solvent such as N-methylpyrrolidone, N,N-dimethylformamide or acetonitrile using a base such as sodium carbonate, potassium carbonate, cesium carbonate or sodium hydroxide. The esterification reaction may be performed in a solvent such as methylene chloride or acetonitrile using a base such as triethylamine, diisopropylethylamine, pyridine or N,N-dimethylaminopyridine.

The second step is hydrolysis of intermediate (B) with a hydroxide salt (M_{B}⁺OH⁻) into an intermediate (C). Examples of the hydroxide salt (M_{B}⁺OH⁻) include lithium hydroxide, sodium hydroxide, potassium hydroxide, and tetramethylammonium hydroxide. Suitable reaction solvents include tetrahydrofuran (THF), dioxane, water or a mixture thereof.

The third step is an ion exchange between the intermediate (C) having the desired anion structure and a salt (M⁺X_{B}⁻) having the desired cation structure to produce the target compound (D). This ion exchange reaction is readily accomplished by any well-known method, for example, with reference to U.S. Pat. No. 7,511,169 (JP-A 2007-145797).

The synthesis method mentioned above is merely exemplary and the method is not limited thereto.

A resist composition comprising the salt compound having formula (1) or (2) as an acid diffusion inhibitor is improved in LWR and CDU. Although the detail is not well understood, the following reason is presumed.

The salt compound of the invention contains an anion which has a straight structure of 6 or more carbon atoms or a fluorinated chain structure of 4 or more carbon atoms. Due to such a structure included, the acid diffusion inhibitor in the form of the inventive salt compound segregates or localizes in a surface layer of a resist film. A top or surface layer has a higher concentration of acid diffusion inhibitor whereas a bottom layer has a lower concentration of acid diffusion inhibitor. The top or surface layer provides stronger light absorption during exposure so that the concentration of acid generated through photo-decomposition is higher in the top or surface layer. As a result, more acid diffusion inhibitor is present in the top layer having a high acid concentration whereas less acid diffusion inhibitor is present in the bottom layer having a low acid concentration. This distribution is effective for suppressing the diffusion of generated acid, leading to a tailored pattern profile and improvements in lithography properties such as LWR and CDU.

Patent Document 2 discloses an acid diffusion inhibitor in the form of a fluoroalkyl chain-containing carboxylic acid salt, represented by the following formula.

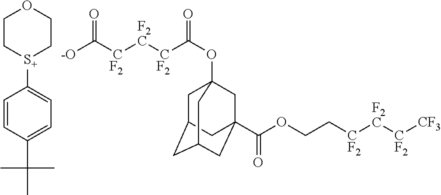

Although this compound is considered to exert the segregation effect toward a surface layer, its lithography performance is inferior to the inventive acid diffusion inhibitor.

Although the detail is not well understood, the following reason is presumed. The compound of the above formula has the structure that the anion site is bonded to the anion main framework via an ester bond. Since the ester structure is a fluorinated alkyl carboxylic acid ester, it is expected that the compound is weak to an alkaline developer, indicating a possibility that the compound is decomposed during development. The decomposed products from alkaline development adversely affect dissolution contrast, the resist film surface layer in the unexposed region is dissolved due to a polarity switch by alkaline development, and consequently, the lithography performance is degraded. In contrast, the anion site of the inventive acid diffusion inhibitor is bonded to the benzene ring or anion main framework directly or via an ether bond or amide bond, which bond is resistant to (i.e., undecomposable in) alkaline developer. Any loss of dissolution contrast during development is avoided. Better lithography performance is thus available.

Patent Document 3 discloses an acid diffusion inhibitor of fluoroalkyl chain-containing amine type, represented by the following formula.

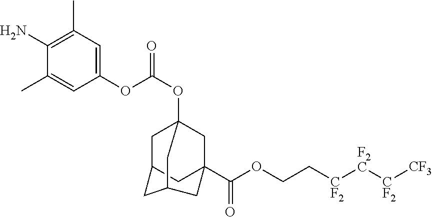 [SMILES: Cc1cc(OC(=O)OC23CC4CC(C2)CC(C(=O)OCCC(F)(F)C(F)(F)C(F)(F)C(F)(F)F)(C4)C3)cc(C)c1N]

Although this compound is also considered to exert the segregation effect toward a surface layer, its dissolution contrast between exposed and unexposed regions is lower than the photo-decomposable salt compound of the invention. In the exposed region, the inventive salt compound allows its cation to be decomposed so that the quenching ability is deactivated by the generated acid. The inventive salt compound thus acts as an acid diffusion inhibitor only in the unexposed region. By contrast, the amine type quencher acts to suppress acid diffusion independent of whether or not the region is exposed, leading to a lower sensitivity and a lower dissolution contrast. It is thus believed that better lithography performance is available from the inventive acid diffusion inhibitor.

When a salt compound containing an iodized anion as represented by formula (1-I) or (2-I) is used as the acid diffusion inhibitor, a higher sensitivity is expectable because of more efficient EUV absorption of iodine atoms.

### Resist Composition

Another embodiment of the invention is a resist composition comprising (A) a base polymer adapted to change its solubility in a developer under the action of an acid, (B) a photoacid generator, (C-1) an acid diffusion inhibitor comprising the inventive salt compound, and (D) an organic solvent as essential components, and if necessary, (C-2) an acid diffusion inhibitor other than the inventive salt compound, (E) a surfactant, and (F) other components.

A further embodiment of the invention is a resist composition comprising (A′) a base polymer adapted to change its solubility in a developer under the action of an acid, the base polymer comprising an acid generating site having a function of generating an acid upon exposure to light as a constituent unit, (C-1) an acid diffusion inhibitor comprising the inventive salt compound, and (D) an organic solvent as essential components, and if necessary, (B) a photoacid generator, (C-2) an acid diffusion inhibitor other than the inventive salt compound, (E) a surfactant, and (F) other components.

### (A) Base Polymer

Component (A) is a base polymer adapted to change its solubility in a developer under the action of an acid. It is preferably a polymer comprising repeat units having the formula (a) or repeat units having the formula (b), which are also referred to as repeat units (a) and (b), respectively.

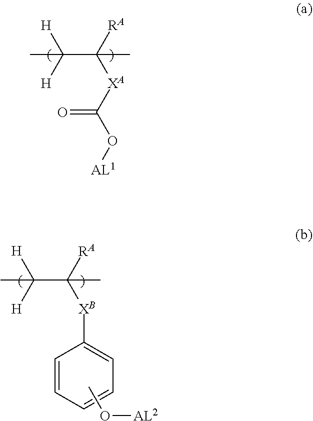

In formulae (a) and (b), R^{A} is hydrogen or methyl. X^{A} is a single bond, phenylene group, naphthylene group or *—C(═O)—O—X^{A1}— wherein the asterisk (*) designates a point of attachment to the carbon atom in the backbone. X^{A1} is a C₁-C₁₅ hydrocarbylene group which may contain at least one moiety selected from hydroxy moiety, ether bond, ester bond and lactone ring. X^{B} is a single bond or ester bond. AL¹ and AL² are each independently an acid labile group. The hydrocarbylene group may be saturated or unsaturated and straight, branched or cyclic.

While the acid labile groups AL¹ and AL² are not particularly limited, suitable acid labile groups include C₄-C₂₀ tertiary hydrocarbyl groups, trihydrocarbylsilyl groups in which each hydrocarbyl moiety has 1 to 6 carbon atoms, and C₄-C₂₀ oxoalkyl groups. With respect to the structure of these acid labile groups, reference should be made to U.S. Pat. No. 9,164,384 (JP-A 2014-225005, paragraphs [0016]-[0035]).

Acid labile groups having the following formula (L1) are preferred as AL¹ and AL².

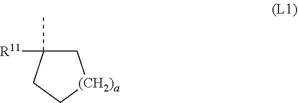

In formula (L1), R¹¹ is a C₁-C₇ hydrocarbyl group in which —CH₂— may be replaced by —O—, and “a” is 1 or 2.

Of the acid labile groups AL¹ and AL², the following groups are most preferred.

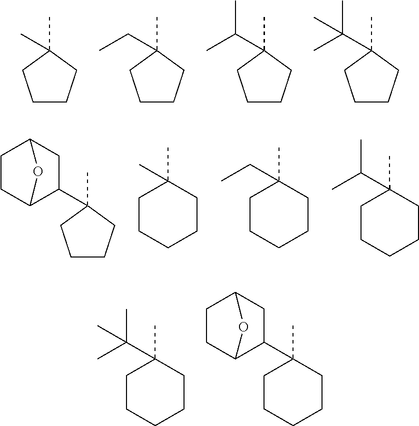

Examples of the structure having formula (a) wherein X^{A} is a variant include the structures described in U.S. Pat. No. 9,164,384 (JP-A 2014-225005, paragraph [0015]). Of these, preferred structures are shown below. Herein R^{A} and AL¹ are as defined above.

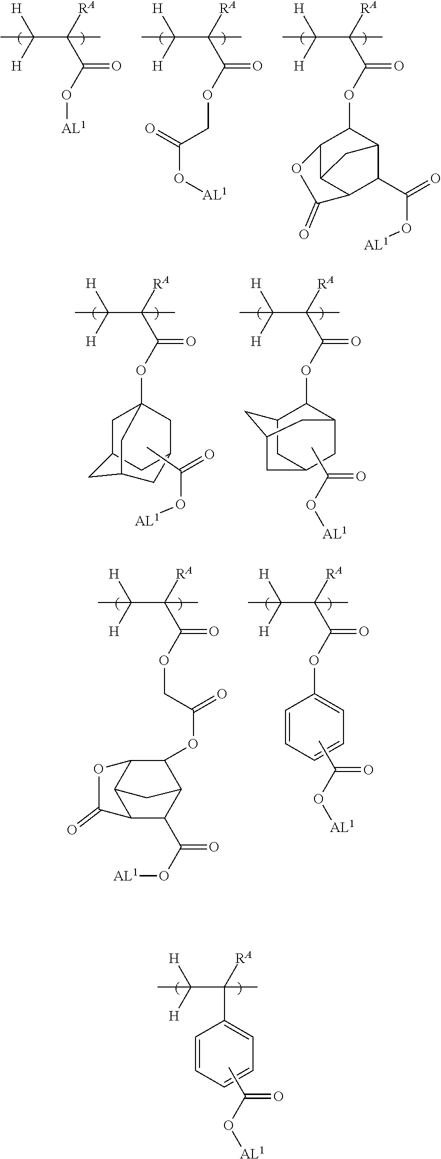

Examples of the repeat unit (a) are given below, but not limited thereto. Herein R^{A} is as defined above.

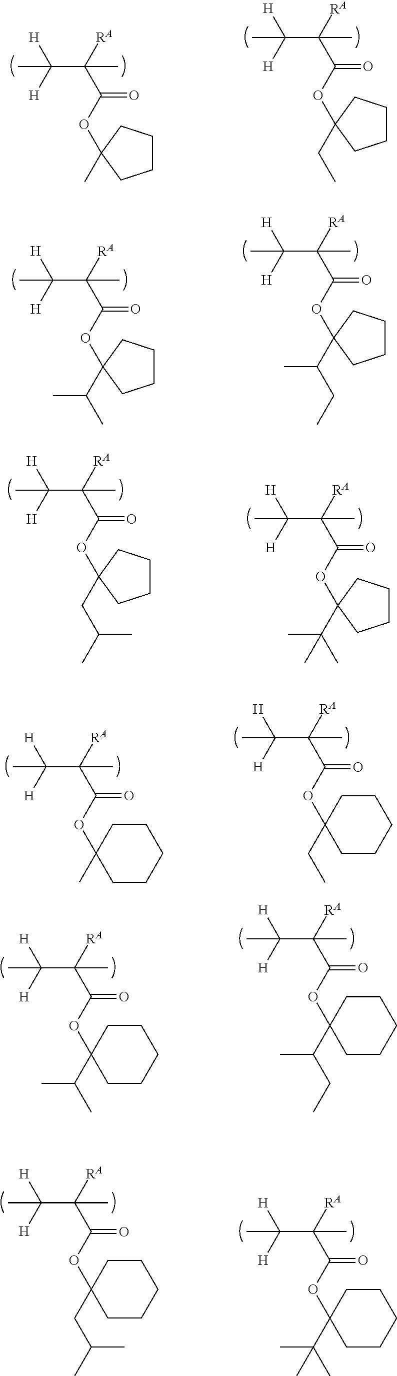

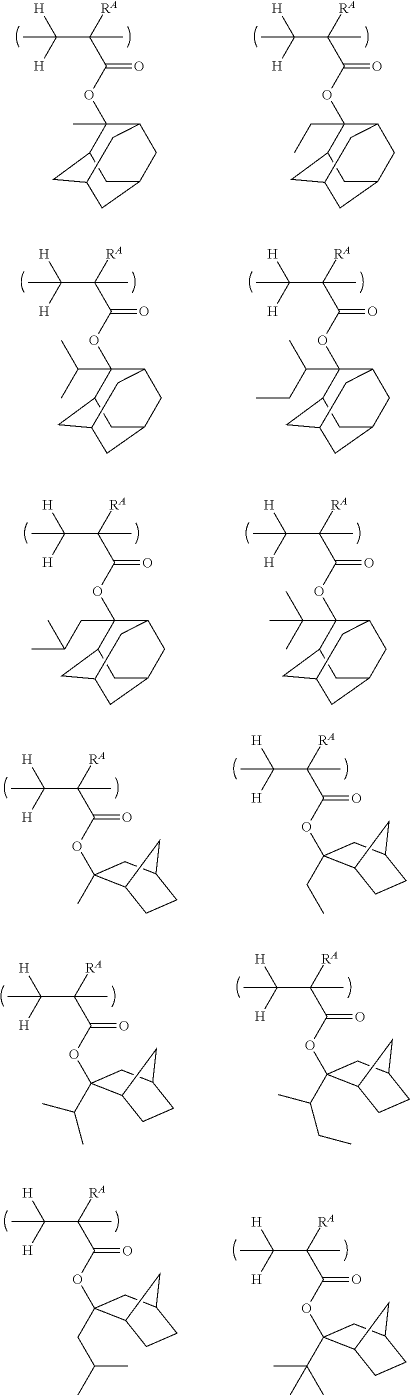

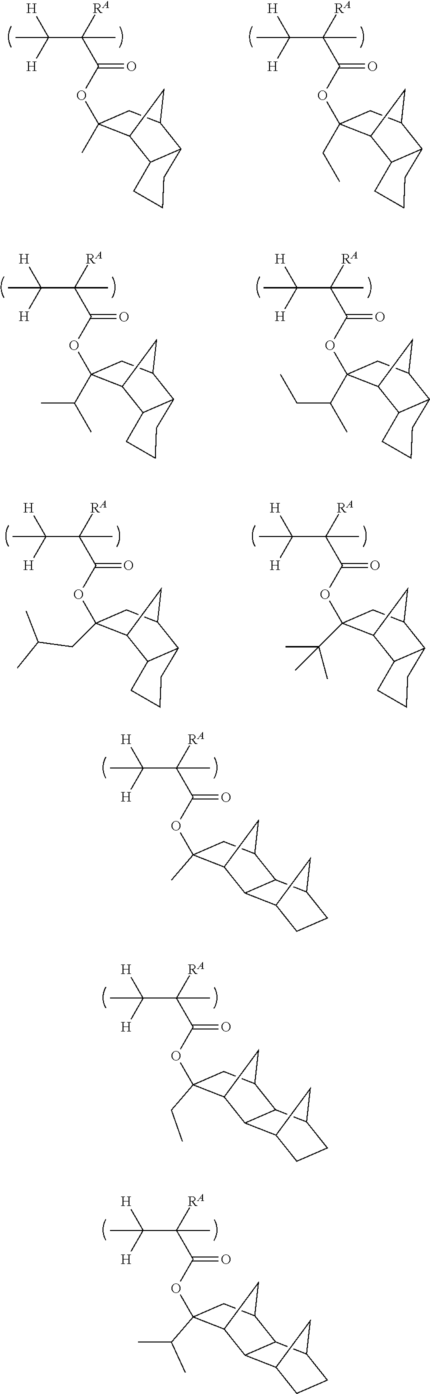

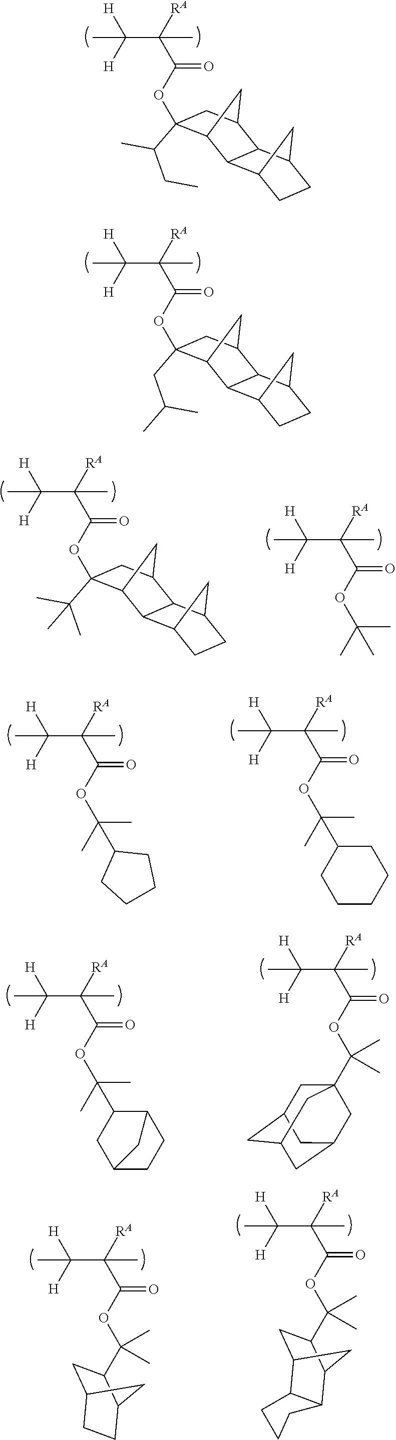

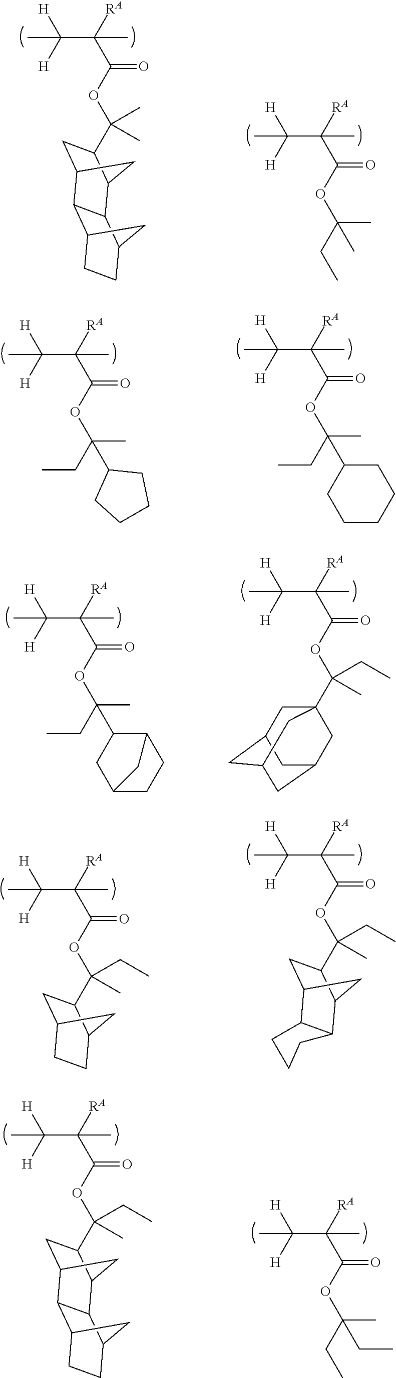

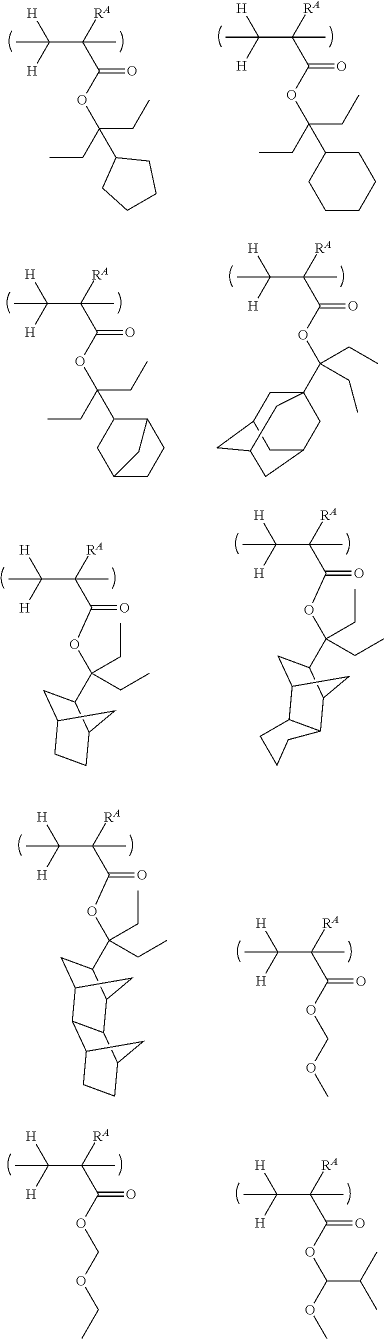

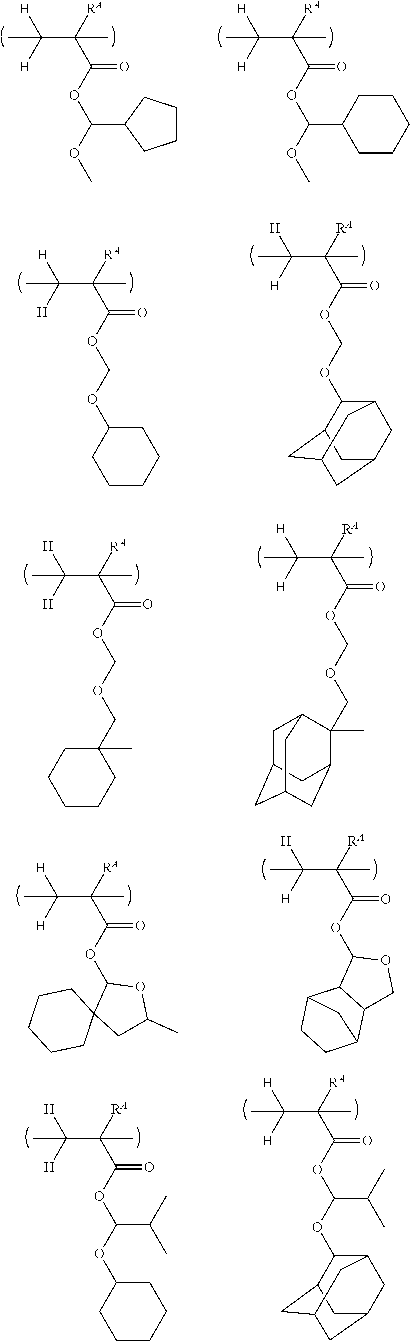

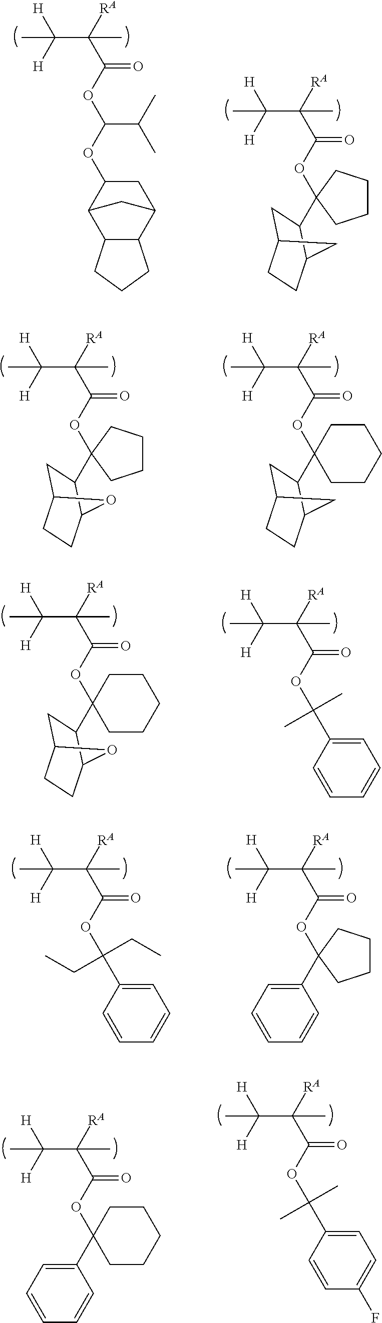

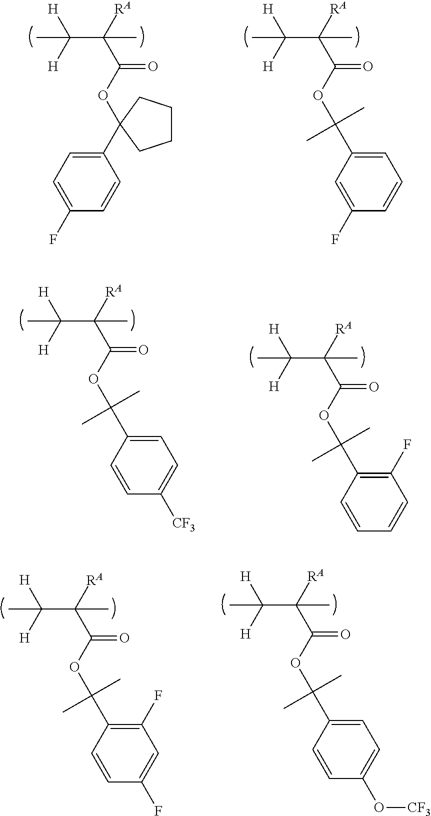

Examples of the repeat unit (b) are given below, but not limited thereto. Herein R^{A} is as defined above.

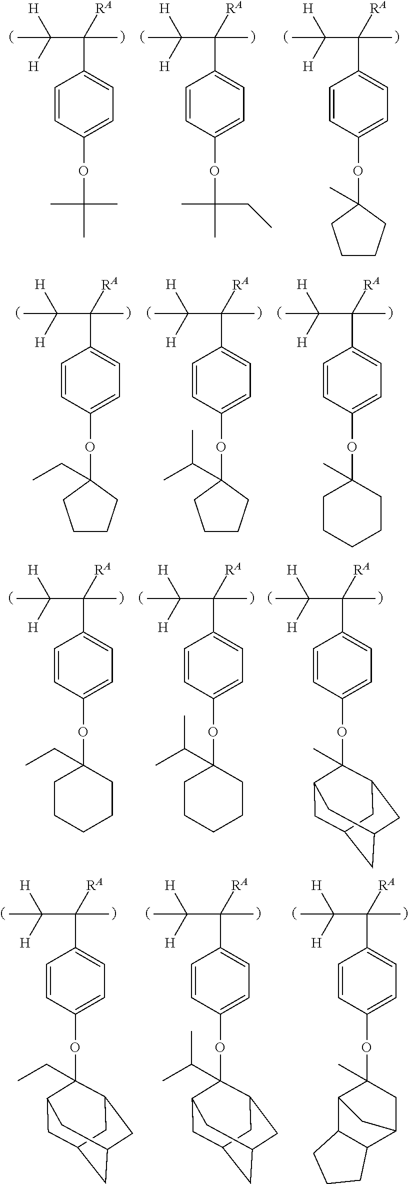

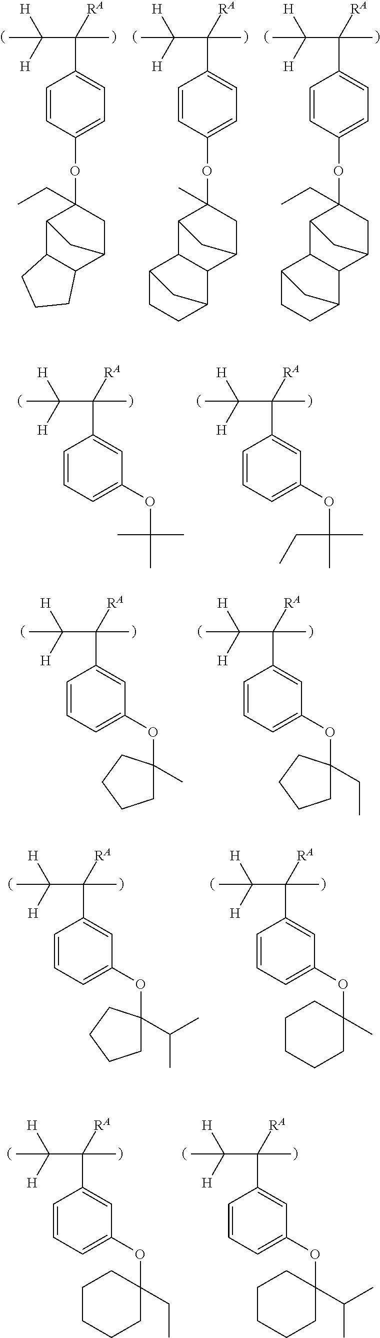

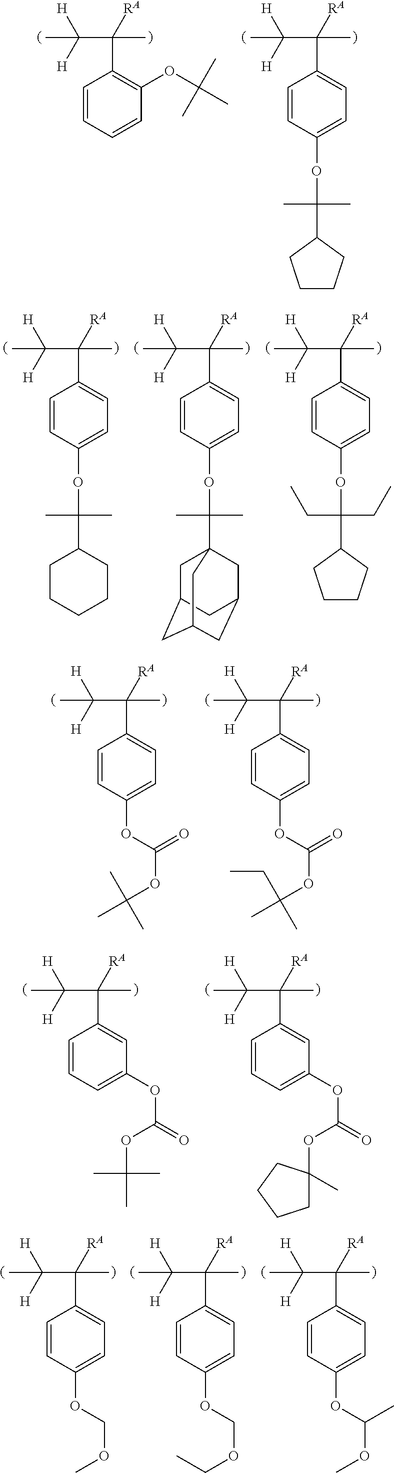

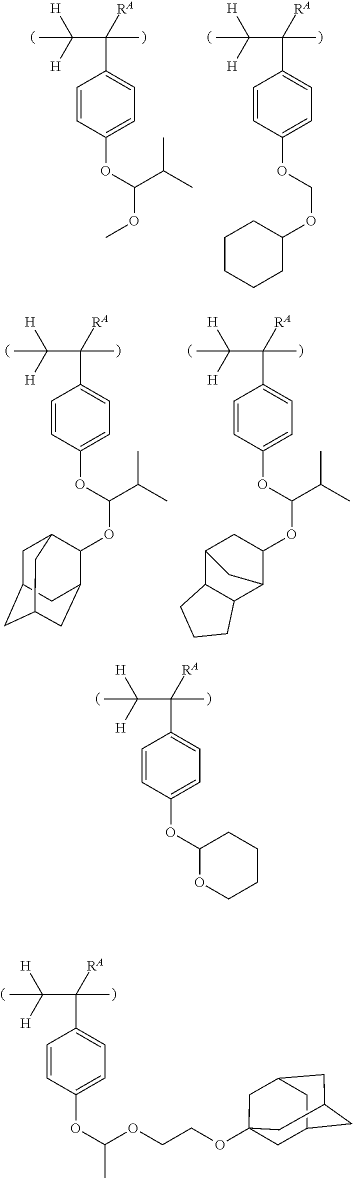

Although the above examples correspond to the unit wherein X^{A} or X^{B} is a single bond, combinations with similar acid labile groups are possible where X^{A} or X^{B} is other than a single bond. Examples of the units wherein X^{A} is other than a single bond are as exemplified above. Examples of the units wherein X^{B} is an ester bond correspond to the above-exemplified units wherein the single bond between the backbone and the benzene ring is replaced by an ester bond.

The base polymer may further comprise repeat units having the formula (c), which are also referred to as repeat units (c).

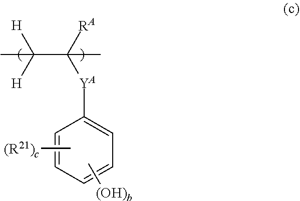

In formula (c), R^{A} is hydrogen or methyl. Y^{A} is a single bond or ester bond.

In formula (c), R²¹ is fluorine, iodine or a C₁-C₁₀ hydrocarbyl group. The hydrocarbyl group may be saturated or unsaturated and straight, branched or cyclic. Examples include alkyl groups such as methyl, ethyl, n-propyl, isopropyl, n-butyl, sec-butyl, tert-butyl, n-pentyl, n-hexyl, n-octyl, n-nonyl, and n-decyl; cyclic saturated hydrocarbyl groups such as cyclopentyl, cyclohexyl, and adamantyl; aryl groups such as phenyl; and combinations thereof. A constituent —CH₂— in the hydrocarbyl group may be replaced by —O— or —C(═O)—. The constituent —CH₂— in the hydrocarbyl group may be one bonded to a carbon atom on the benzene ring in formula (c). Examples of the substituted hydrocarbyl group include, but are not limited to, methoxy, ethoxy, propoxy, butoxy, phenoxy, 2-methoxyethoxy, acetyl, ethylcarbonyl, hexylcarbonyl, acetoxy, ethylcarbonyloxy, propylcarbonyloxy, pentylcarbonyloxy, hexylcarbonyloxy, heptylcarbonyloxy, methoxymethylcarbonyloxy, (2-methoxyethoxy)methylcarbonyloxy, methyloxycarbonyl, ethyloxycarbonyl, hexyloxycarbonyl, phenyloxycarbonyl, acetoxymethyl, phenoxymethyl, and methoxycarbonyloxy. Preferably R²¹ is fluorine, iodine, methyl, acetyl or methoxy.

In formula (c), b is an integer of 1 to 5, c is an integer of 0 to 4, and b+c is from 1 to 5. Preferably b is 1, 2 or 3, and c is 0, 1 or 2.

The repeat unit (c) serves to improve the adhesion to the substrate or the underlay film. Since the repeat unit (c) has a phenolic hydroxy group with high acidity, it promotes the action of an acid generated upon exposure, contributing to a higher sensitivity, and becomes a proton source to the acid generated upon EUV exposure, from which an improvement in sensitivity is expectable.

Examples of the repeat unit (c) are given below, but not limited thereto. Herein R^{A} is as defined above.

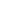

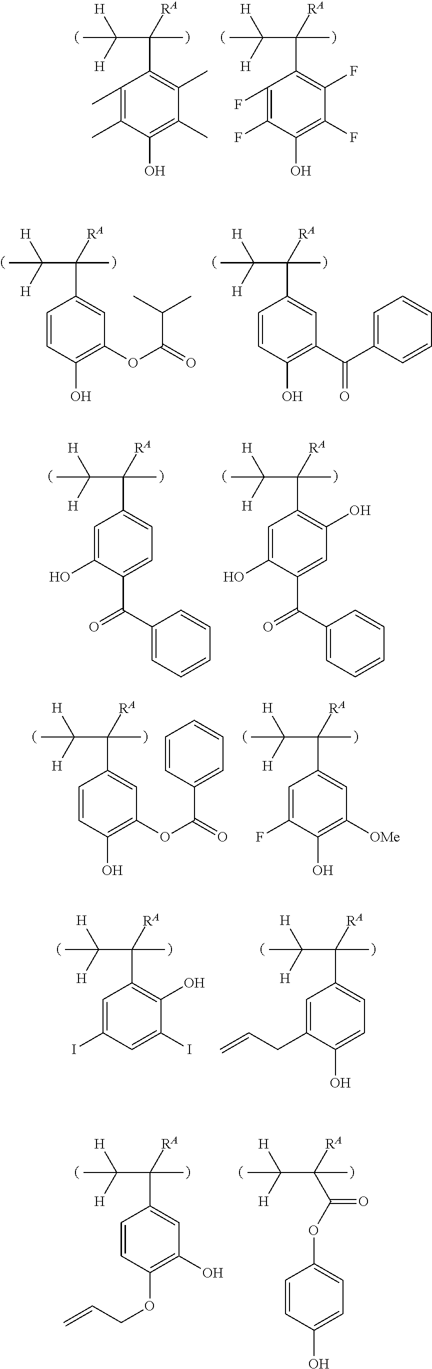

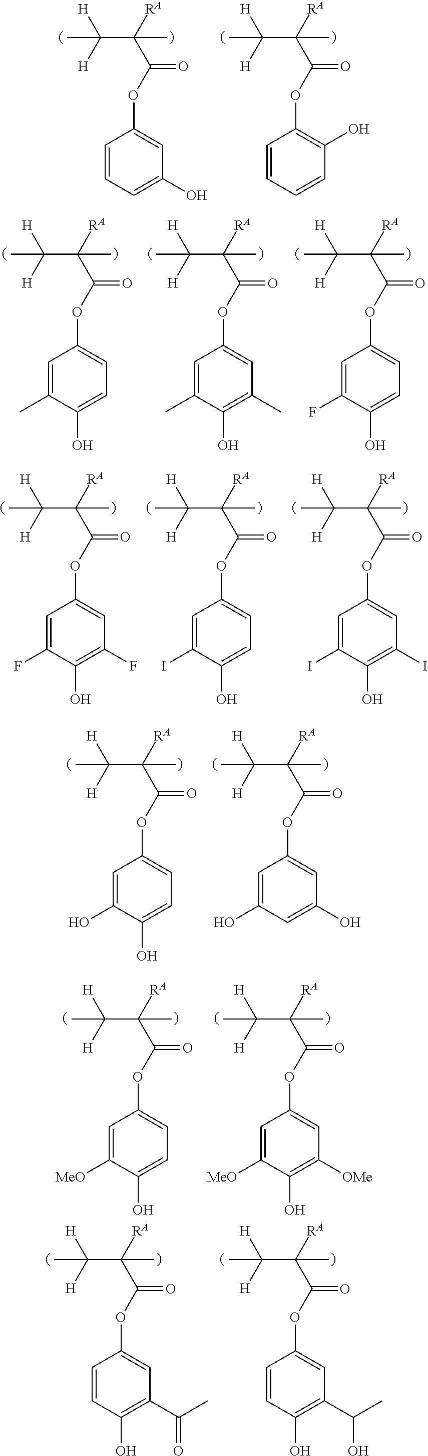

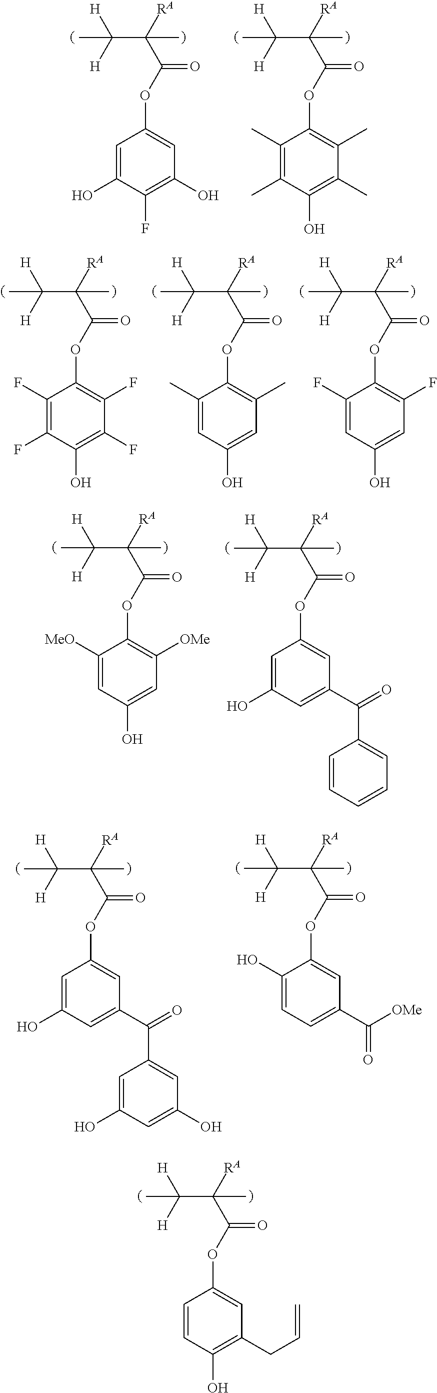

Of the above repeat units (c), the following units are preferred. Herein R^{A} is as defined above.

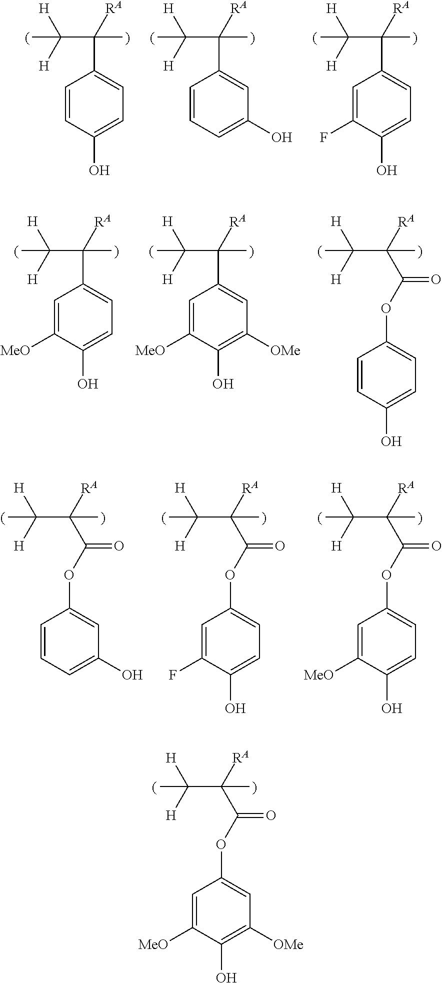

The base polymer may further comprise repeat units having the formula (d1), (d2), (d3) or (d4), which are also referred to as repeat units (d1) to (d4), respectively.

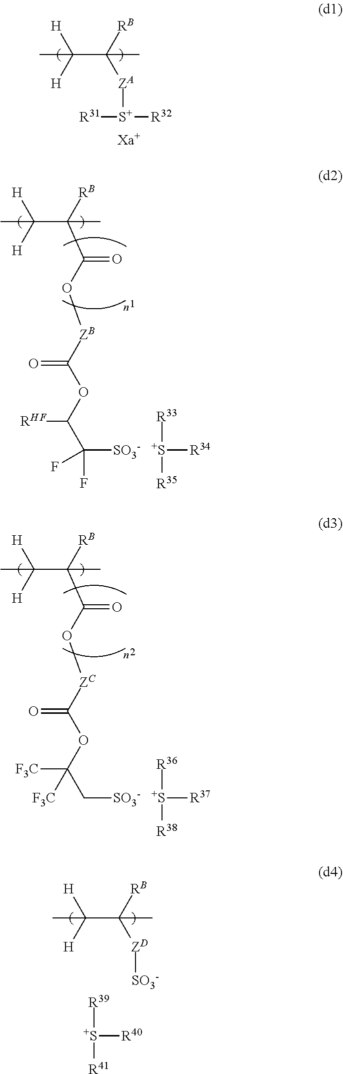

In formulae (d1) to (d4), R^{B} is hydrogen, fluorine, methyl or trifluoromethyl. Z^{A} is a single bond, phenylene, —O—Z^{A1}—, —C(═O)—O—Z^{A1}— or —C(═O)—NH—Z^{A1}—, wherein Z^{A1} is a C₁-C₂₀ hydrocarbylene group which may contain a heteroatom. Z^{B} and Z^{C} are each independently a single bond or a C₁-C₂₀ hydrocarbylene group which may contain a heteroatom. Z^{D} is a single bond, methylene, ethylene, phenylene, fluorinated phenylene, —O—Z^{D1}—, —C(═O)—O—Z^{D1}— or —C(═O)—N(H)—Z^{D1}—, wherein Z^{D1} is an optionally substituted phenylene group.

The hydrocarbylene group represented by Z^{A1} may be saturated or unsaturated and straight, branched or cyclic. Examples thereof include alkanediyl groups such as methylene, ethane-1,1-diyl, ethane-1,2-diyl, propane-1,2-diyl, propane-1,3-diyl, butane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, and 2,2-dimethylpropane-1,3-diyl; cyclic saturated hydrocarbylene groups such as cyclopentanediyl, cyclohexanediyl, norbornanediyl, and adamantanediyl; alkenediyl groups such as ethene-1,2-diyl, 1-propene-1,3-diyl, 2-butene-1,4-diyl, and 1-methyl-1-butene-1,4-diyl; unsaturated alicyclic hydrocarbylene groups such as 2-cyclohexene-1,4-diyl; aromatic hydrocarbylene groups such as phenylene and naphthylene, and combinations thereof. In the hydrocarbylene group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, carbonyl moiety, ether bond, ester bond, sulfonic ester bond, carbonate bond, lactone ring, sultone ring, carboxylic anhydride (—C(═O)—O—C(═O)—) or haloalkyl moiety.

The hydrocarbylene groups represented by Z^{B} and Z^{C} may be saturated or unsaturated and straight, branched or cyclic. Examples thereof are as exemplified above for the hydrocarbylene group Z^{A1}. Preferably Z^{B} and Z^{C} each are a single bond, adamantanediyl or phenylene.

In formulae (d1) to (d4), R³¹ to R⁴¹ are each independently a C₁-C₂₀ hydrocarbyl group which may contain a heteroatom. The hydrocarbyl group may be saturated or unsaturated and straight, branched or cyclic. Examples include alkyl groups such as methyl, ethyl, n-propyl, isopropyl, n-butyl, sec-butyl, tert-butyl, n-pentyl, n-hexyl, n-octyl, 2-ethylhexyl, n-nonyl, n-decyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2.6}]decanyl, adamantyl, adamantylmethyl; alkenyl groups such as vinyl, allyl, propenyl, butenyl, and hexenyl; unsaturated alicyclic hydrocarbyl groups such as cyclohexenyl; aryl groups such as phenyl, naphthyl, thienyl, 4-hydroxyphenyl, 4-methoxyphenyl, 3-methoxyphenyl, 2-methoxyphenyl, 4-ethoxyphenyl, 4-tert-butoxyphenyl, 3-tert-butoxyphenyl, 2-methylphenyl, 3-methylphenyl, 4-methylphenyl, 4-ethylphenyl, 4-tert-butylphenyl, 4-n-butylphenyl, 2,4-dimethylphenyl, 2,4,6-triisopropylphenyl, methylnaphthyl, ethylnaphthyl, methoxynaphthyl, ethoxynaphthyl, n-propoxynaphthyl, n-butoxynaphthyl, dimethylnaphthyl, diethylnaphthyl, dimethoxynaphthyl, diethoxynaphthyl; aralkyl groups such as benzyl, 1-phenylethyl and 2-phenylethyl, and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, carbonyl moiety, ether bond, ester bond, sulfonic ester bond, carbonate bond, lactone ring, sultone ring, carboxylic anhydride (—C(═O)—O—C(═O)—) or haloalkyl moiety.

Z^{A} and R³¹ to R⁴¹ are preferably of a structure containing a phenyl group which is bonded to S⁺ in the formula.

Any two or more of Z^{A}, R³¹ and R³² may bond together to form a ring with the sulfur atom to which they are attached, any two or more of R³³, R³⁴ and R³⁵, any two or more of R³⁶, R³⁷ and R³⁸, or any two or more of R³⁹, R⁴⁰ and R⁴¹ may bond together to form a ring with the sulfur atom to which they are attached.

In formula (d2), R^{HF} is hydrogen or trifluoromethyl.

In formula (d2), n¹ is 0 or 1, n¹ is 0 when Z^{B} is a single bond. In formula (d3), n² is 0 or 1, n² is 0 when Z^{C} is a single bond.

In formula (d1), Xa⁻ is a non-nucleophilic counter ion. Examples of the non-nucleophilic counter ion include halide ions such as chloride and bromide ions; fluoroalkylsulfonate ions such as triflate, 1,1,1-trifluoroethanesulfonate, and nonafluorobutanesulfonate; arylsulfonate ions such as tosylate, benzenesulfonate, 4-fluorobenzenesulfonate, and 1,2,3,4,5-pentafluorobenzenesulfonate; alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; and methide ions such as tris(trifluoromethylsulfonyl)methide and tris(perfluoroethylsulfonyl)methide. Preferred are anions having the formulae (d1-1) and (d1-2).

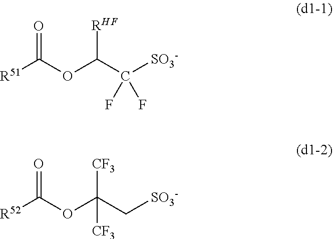

In formulae (d1-1) and (d1-2), R⁵¹ and R⁵² are each independently a C₁-C₄₀ hydrocarbyl group which may contain a heteroatom. R^{HF} is hydrogen or trifluoromethyl.

Examples of the anion having formula (d1-1) include the anions described in JP-A 2014-177407, paragraphs [0100]-[0101] and the anions shown below, but are not limited thereto. Herein R^{HF} is as defined above.

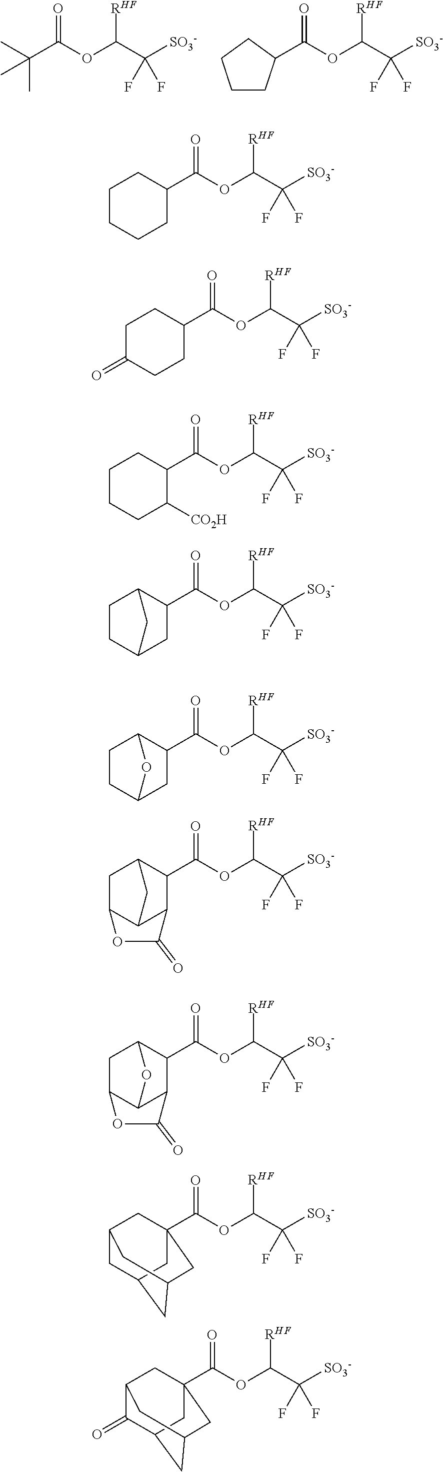

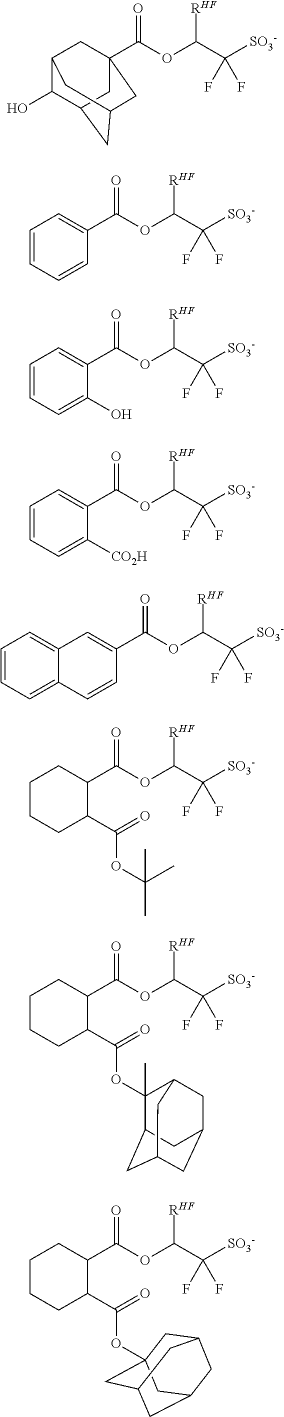

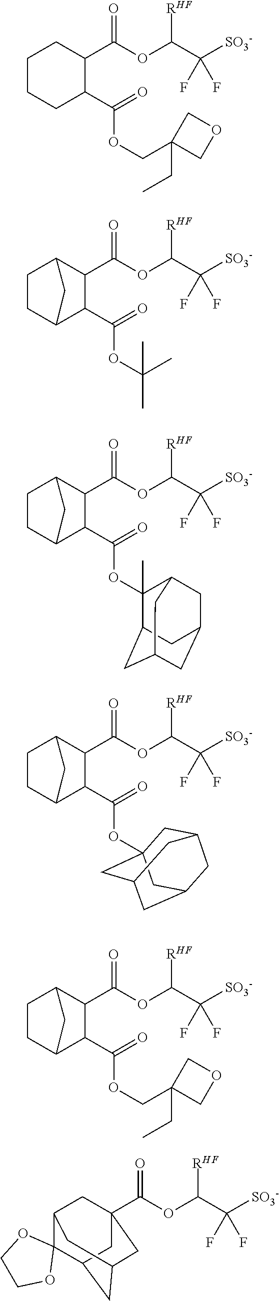

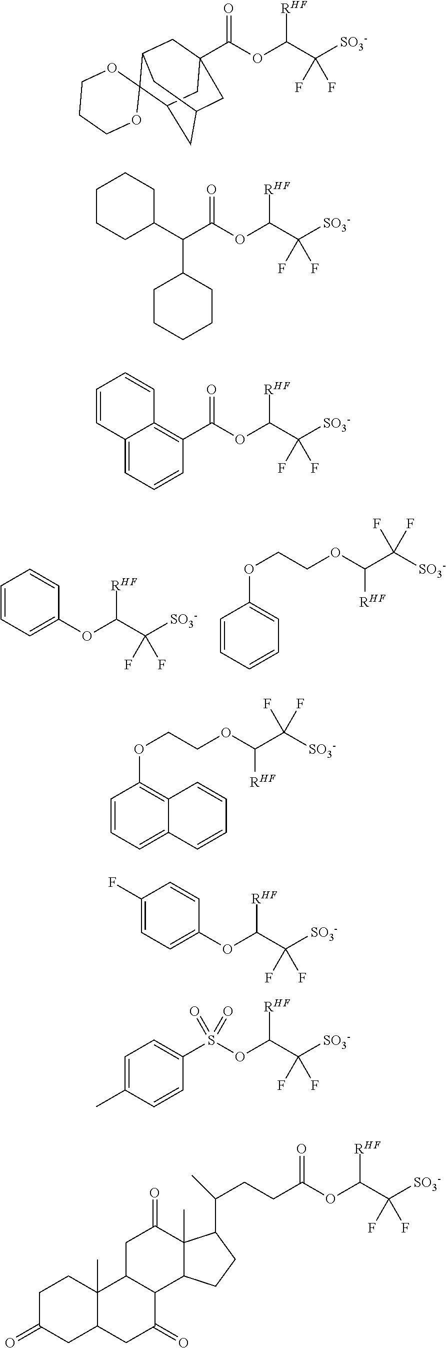

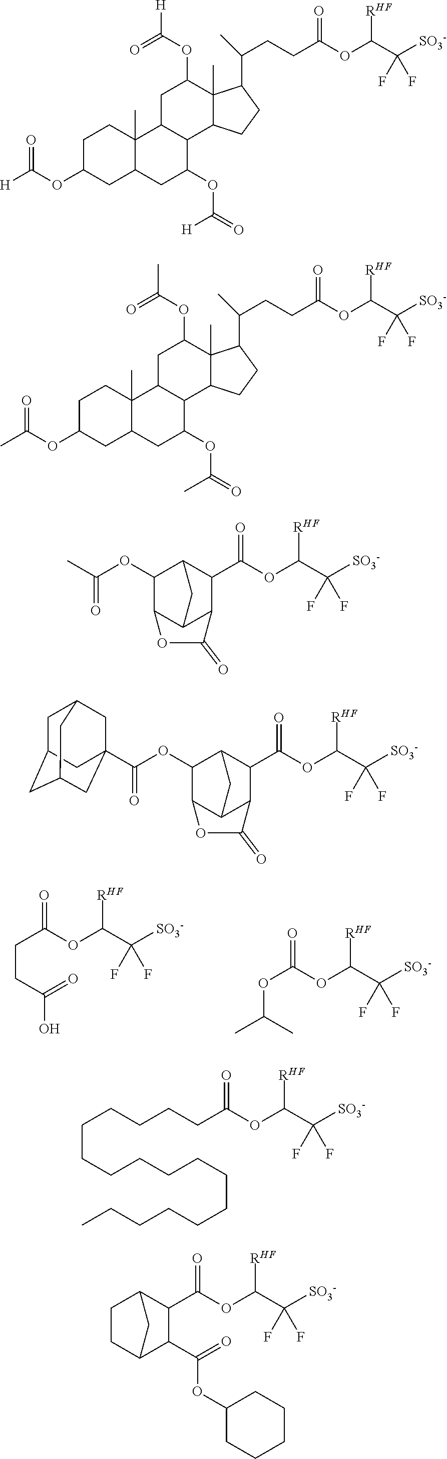

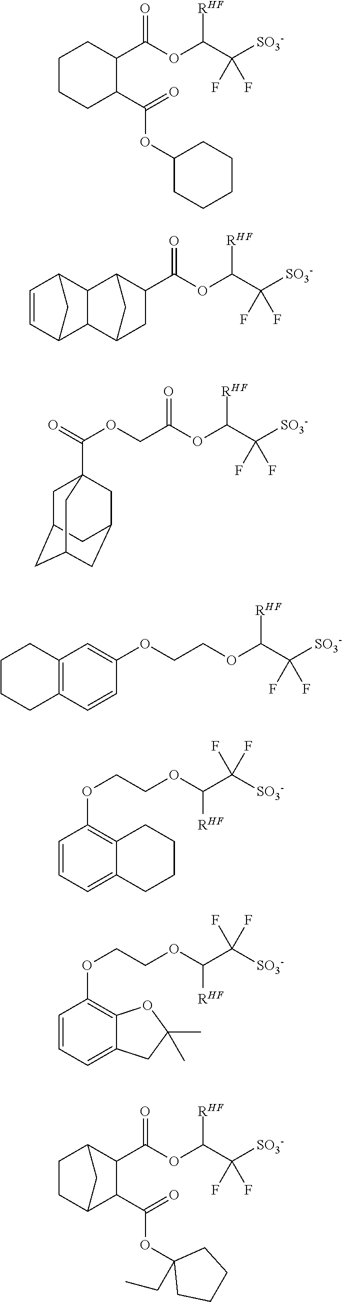

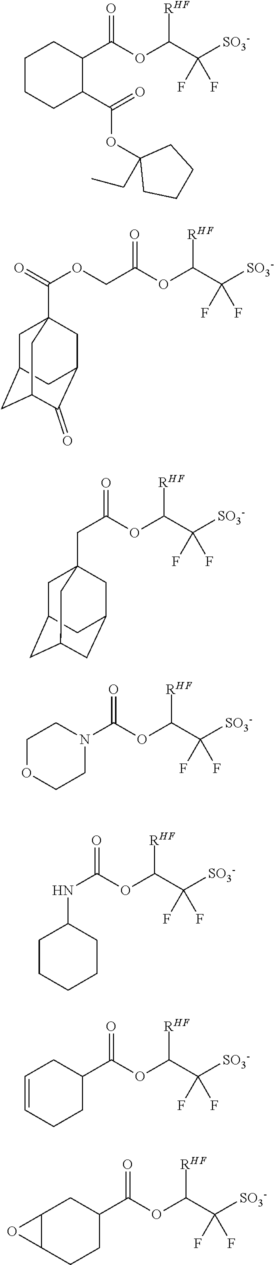

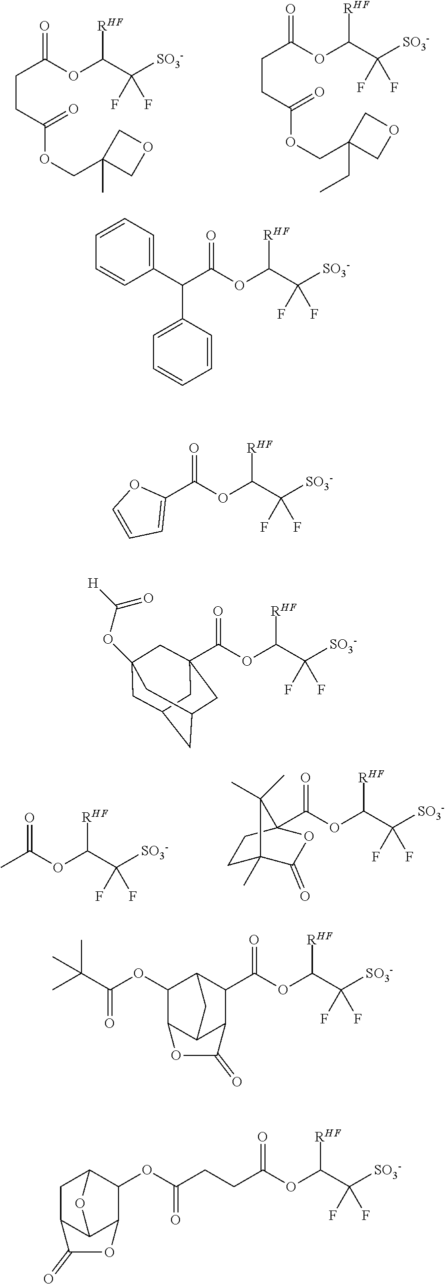

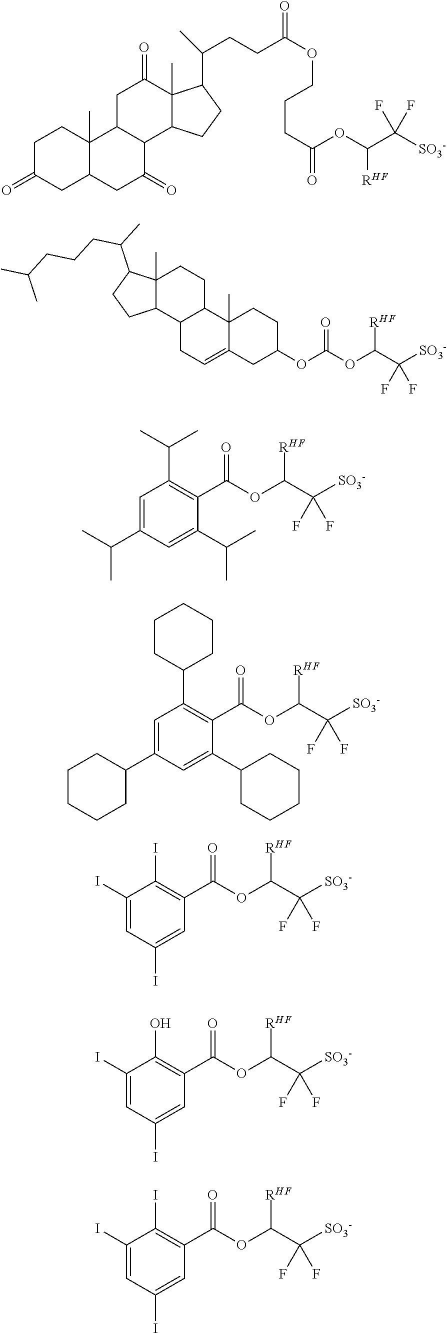

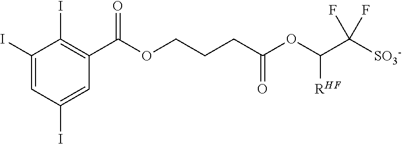

Examples of the anion having formula (d1-2) include the anions described in JP-A 2010-215608, paragraphs [0080]-[0081] and the anions shown below, but are not limited thereto.

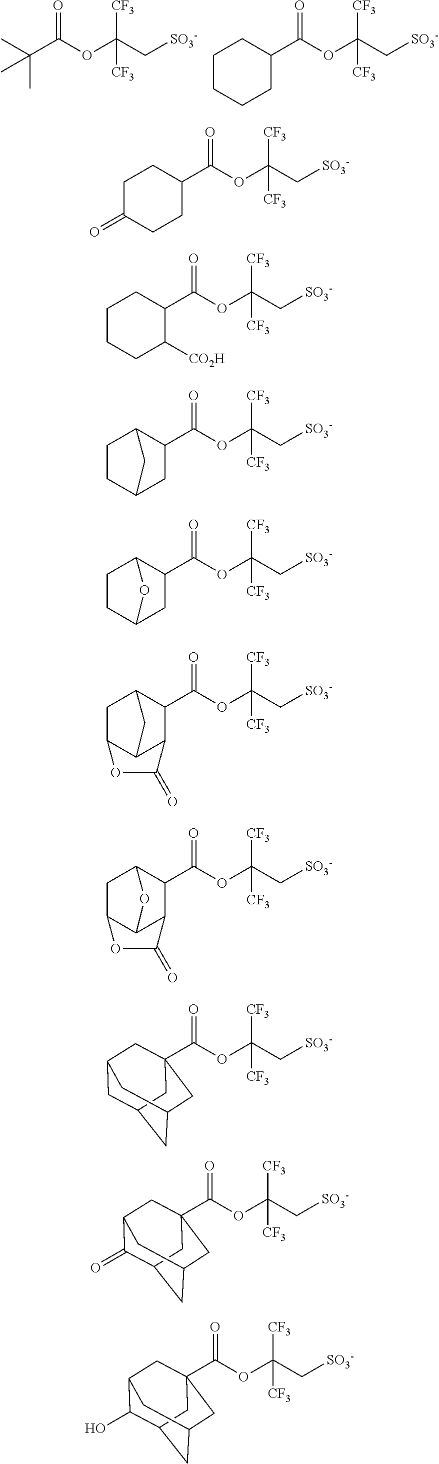

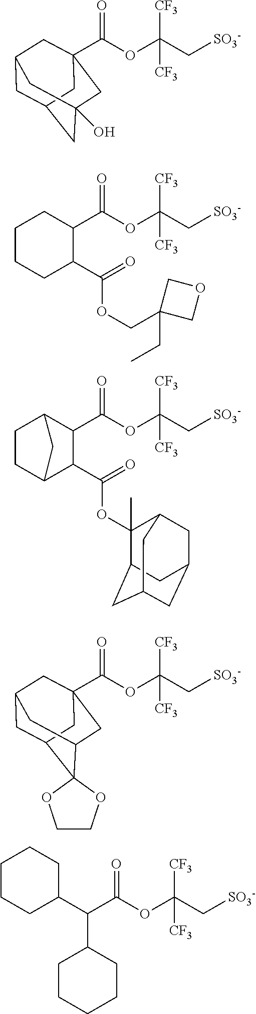

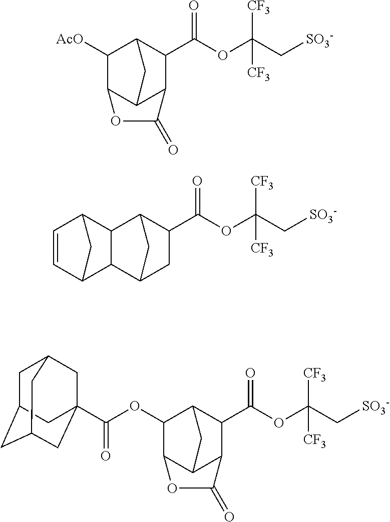

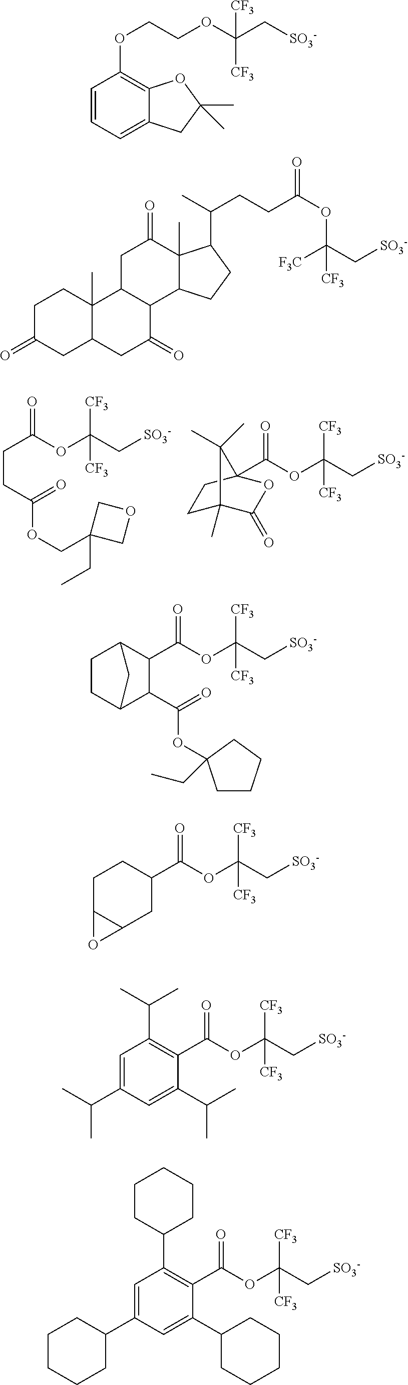

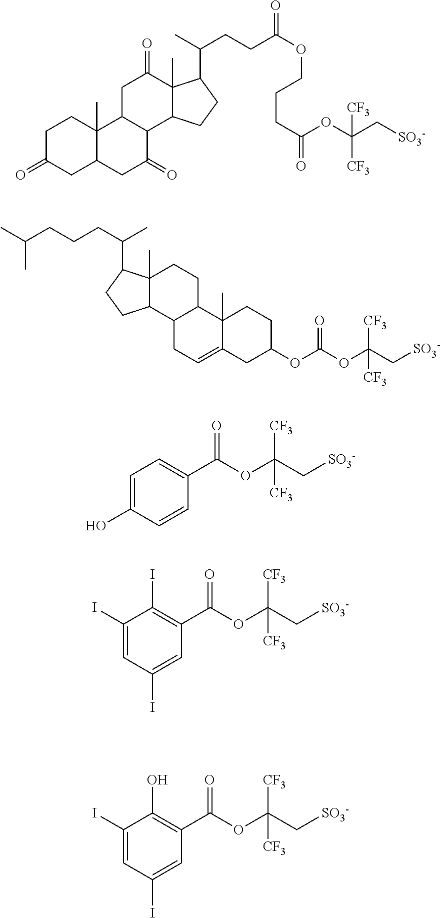

Examples of the anion in repeat unit (d2) include the anions described in JP-A 2014-177407, paragraphs [0021]-[0026]. Exemplary structures of the anion wherein R^{HF} is hydrogen include the anions described in JP-A 2010-116550, paragraphs [0021]-[0028]. Exemplary structures of the anion wherein R^{HF} is trifluoromethyl include the anions described in JP-A 2010-077404, paragraphs [0021]-[0027].

Examples of the anion in repeat unit (d3) correspond to the examples of the anion in repeat unit (d2) wherein —CH(R^{HF})CF₂SO₃⁻ is replaced by —C(CF₃)₂CH₂SO₃⁻.

Preferred examples of the anion in repeat units (d2) to (d4) are given below, but not limited thereto. Herein R^{B} is as defined above.

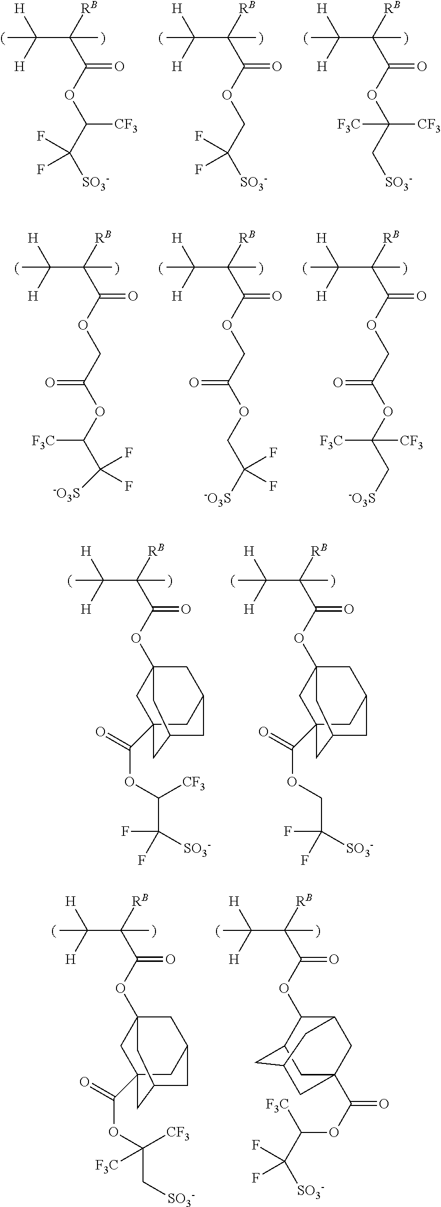

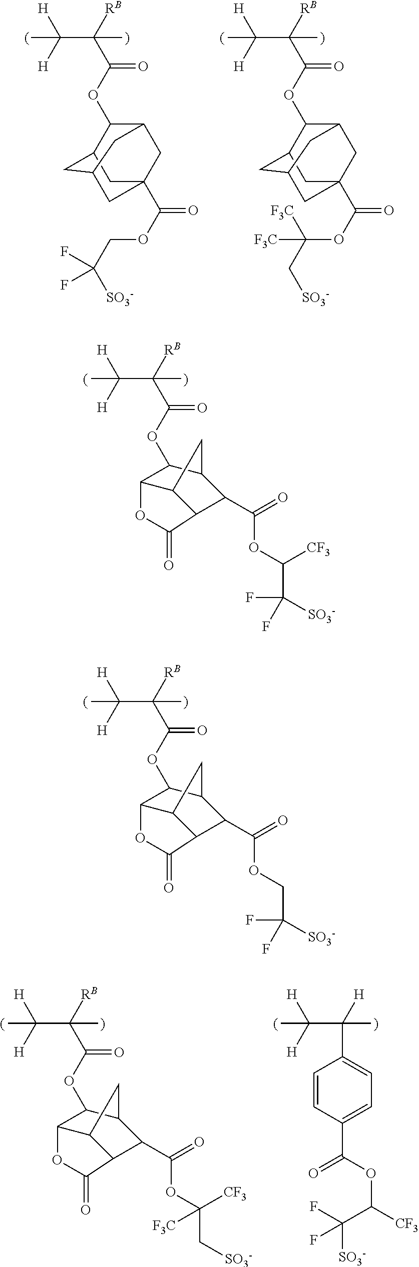

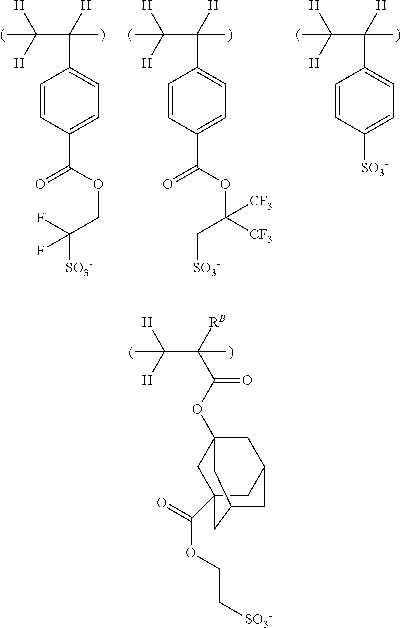

Examples of the sulfonium cation in repeat units (d2) to (d4) include those described in JP-A 2008-158339, paragraph [0223] as well as those exemplified above for the sulfonium cation M⁺ in formula (1) or (2). Of these, the preferred cations are given below, but not limited thereto.

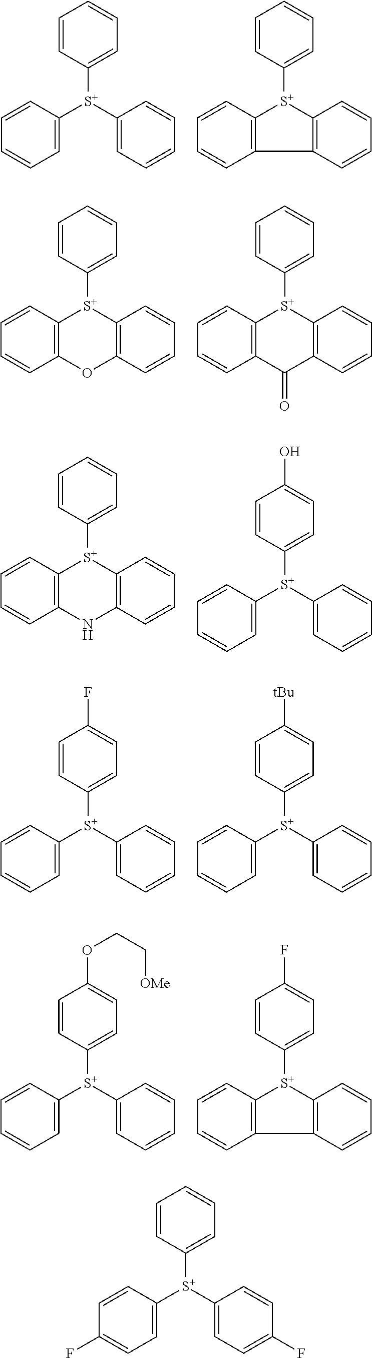

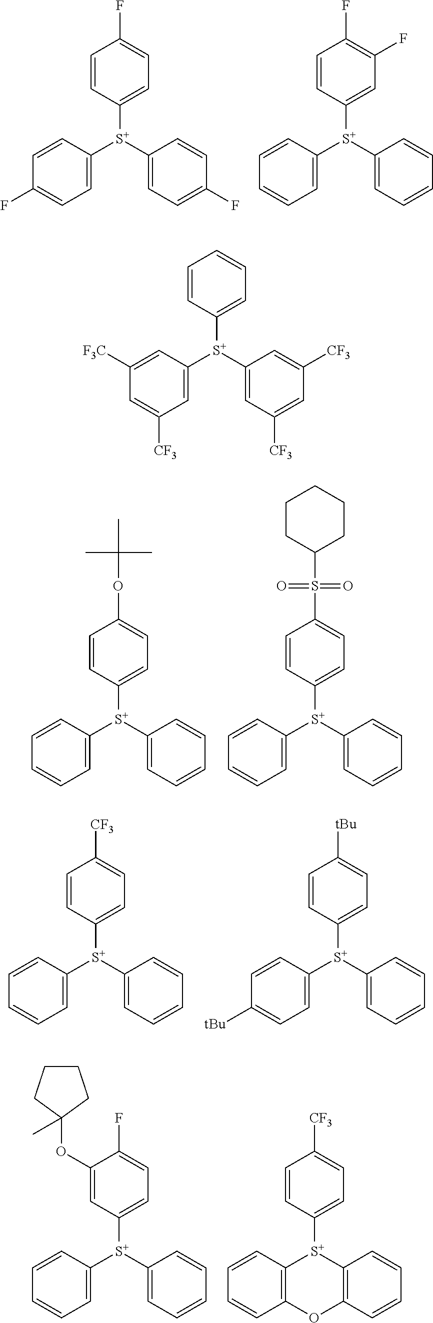

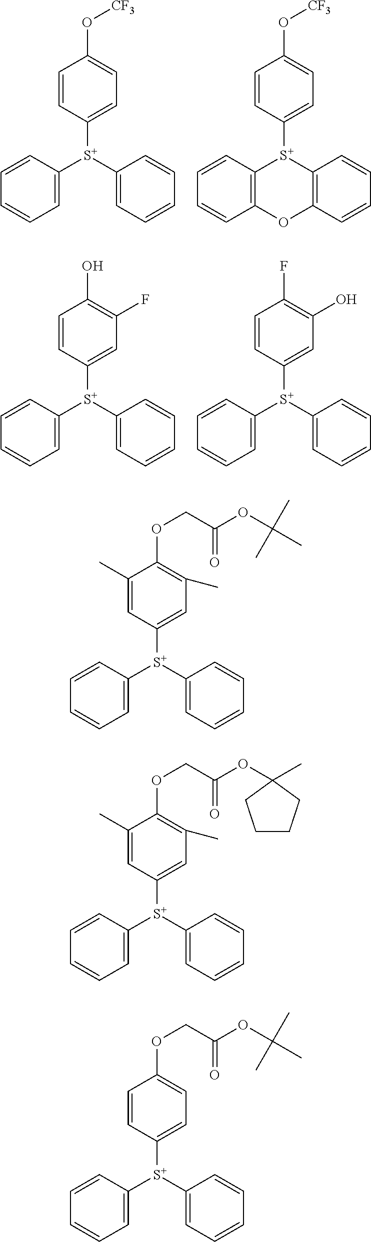

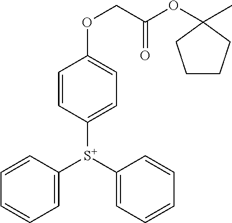

The repeat units (d1) to (d4) have the function of a photoacid generator. On use of a base polymer comprising repeat units (d1) to (d4), a photoacid generator of addition type to be described later may be omitted.

The base polymer may further comprise repeat units (e) containing another adhesive group such as a hydroxy group (other than phenolic hydroxy group), lactone ring, ether bond, ester bond, carbonyl group, cyano group or carboxy group.

Examples of the repeat units (e) are given below, but not limited thereto. Herein R^{A} is as defined above.

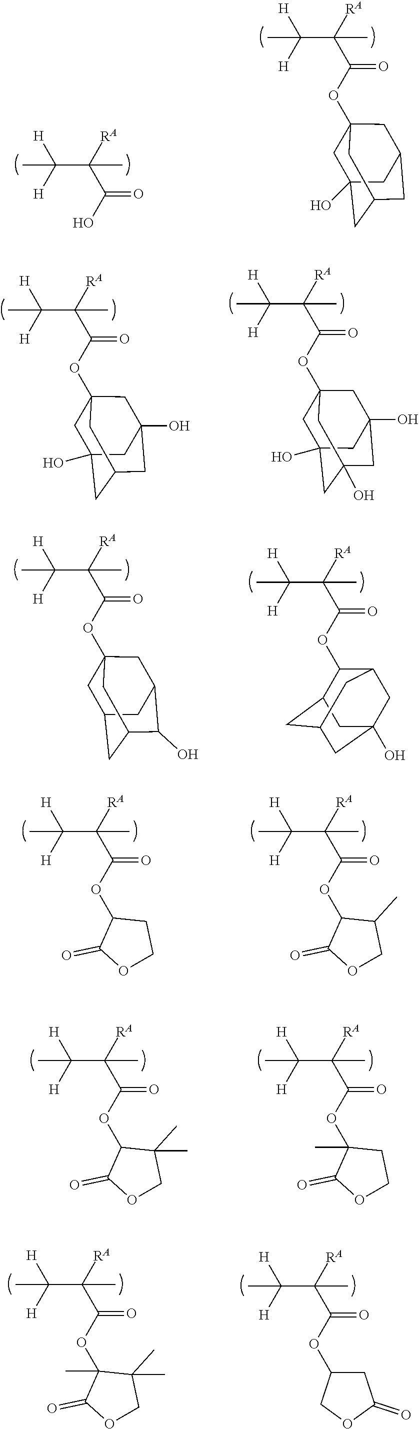

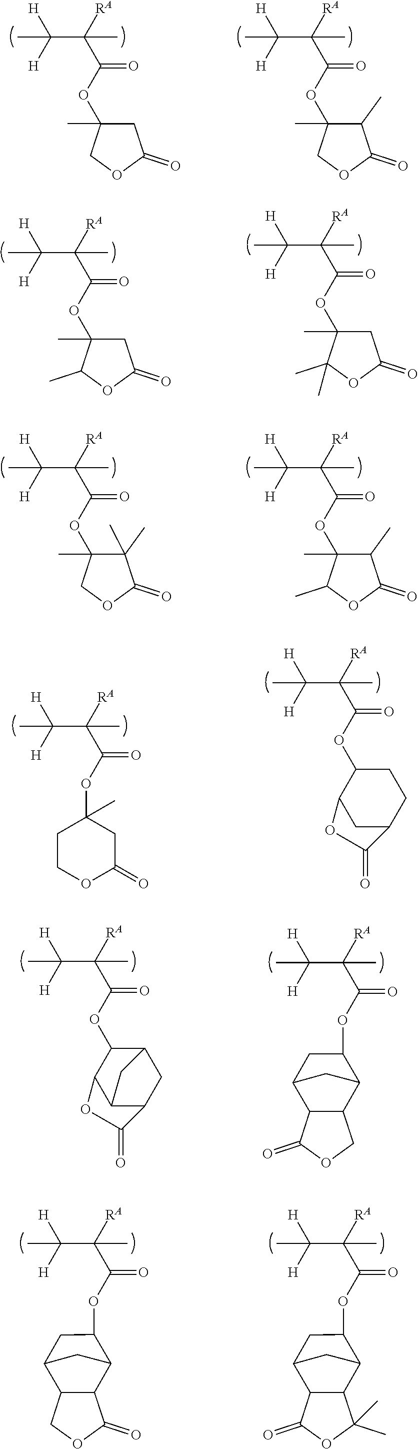

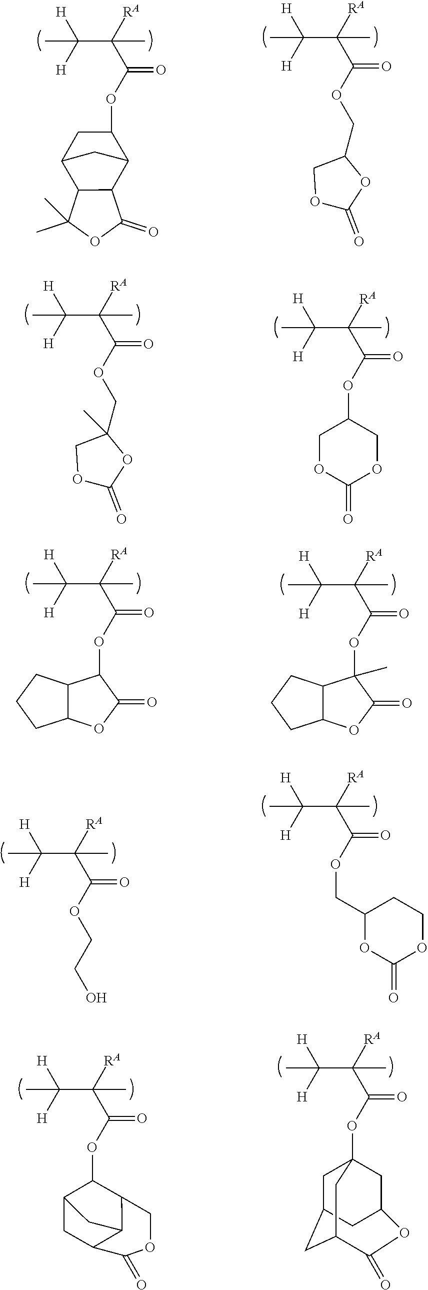

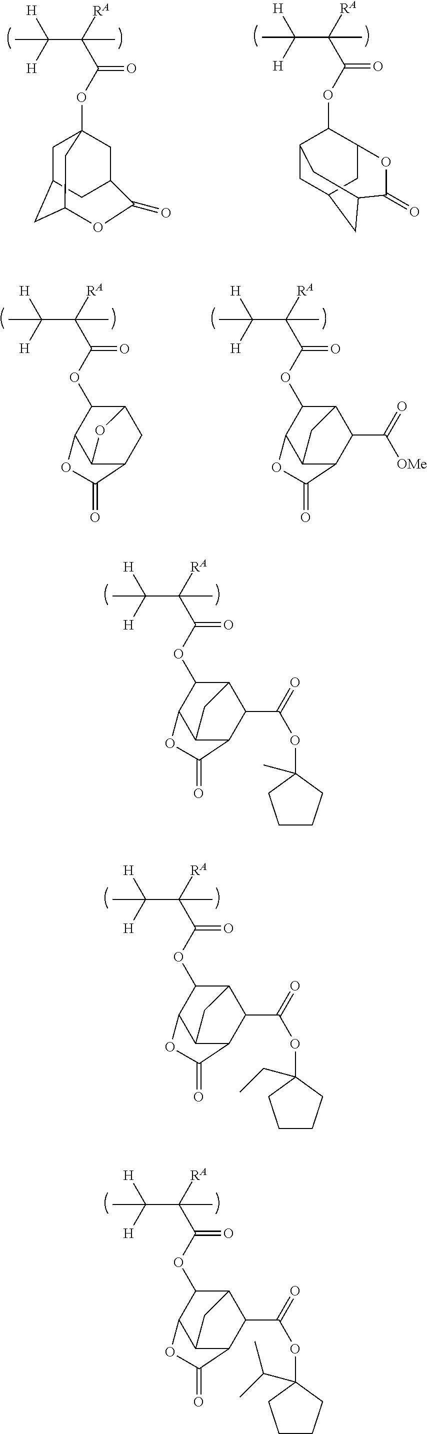

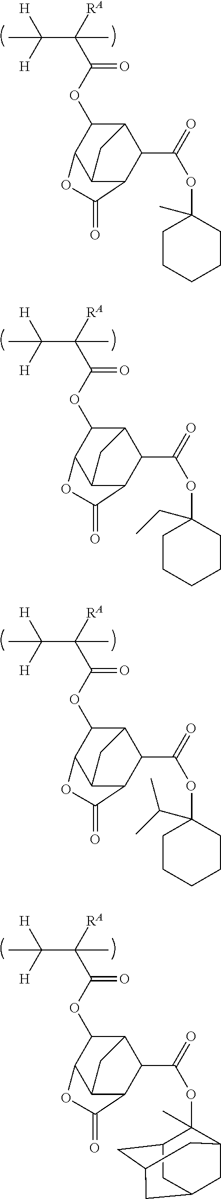

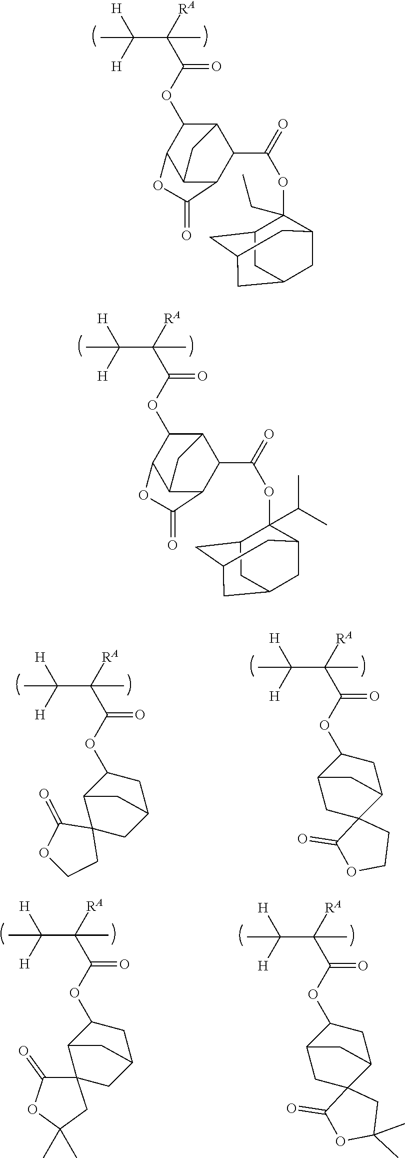

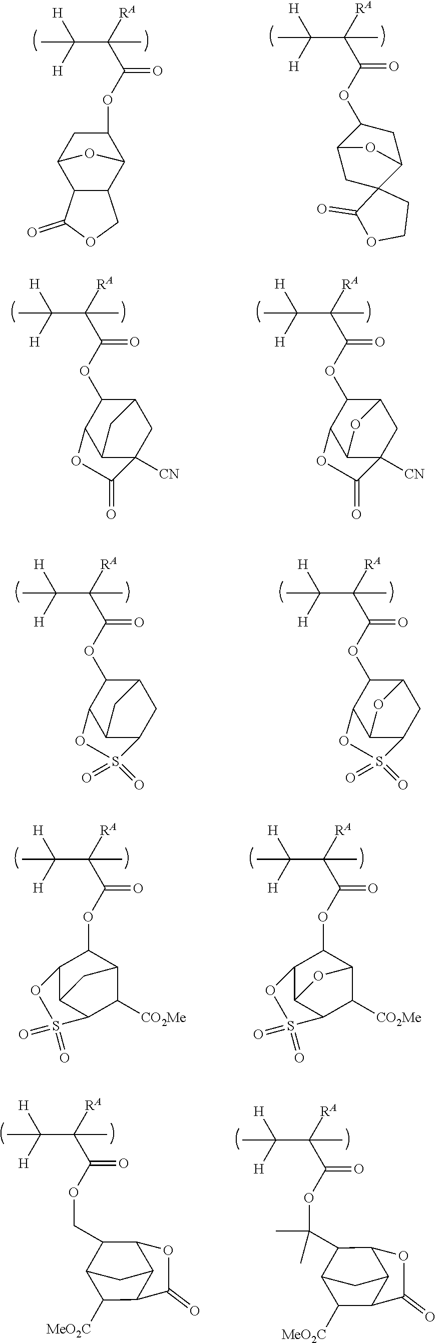

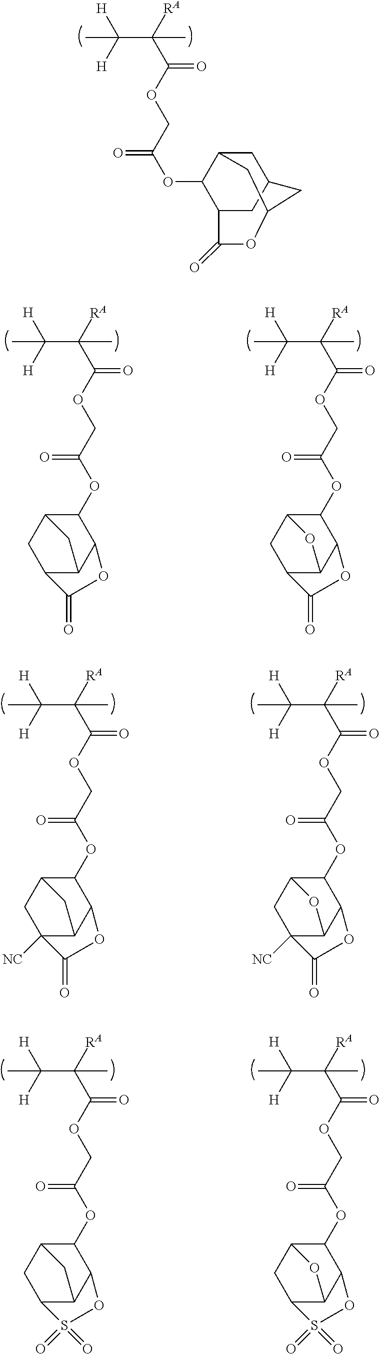

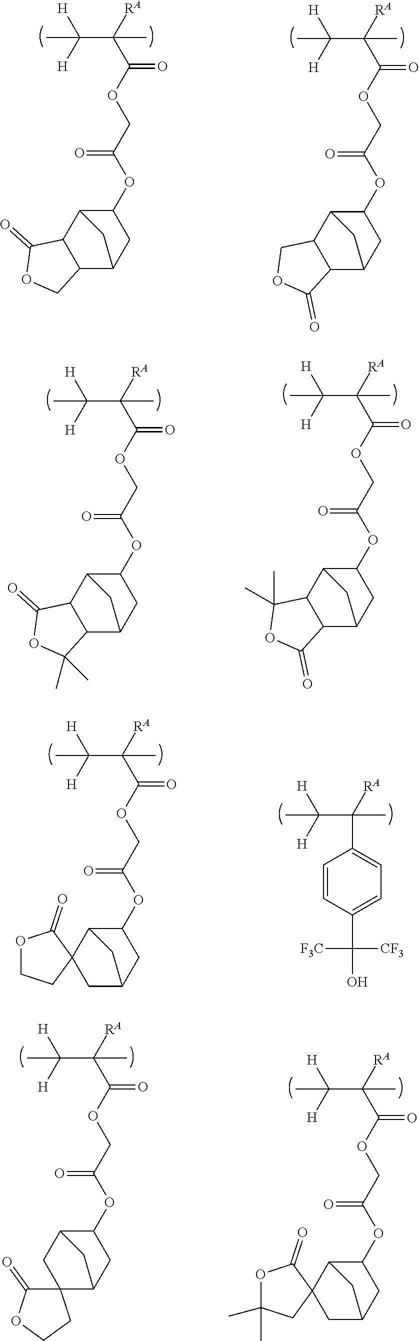

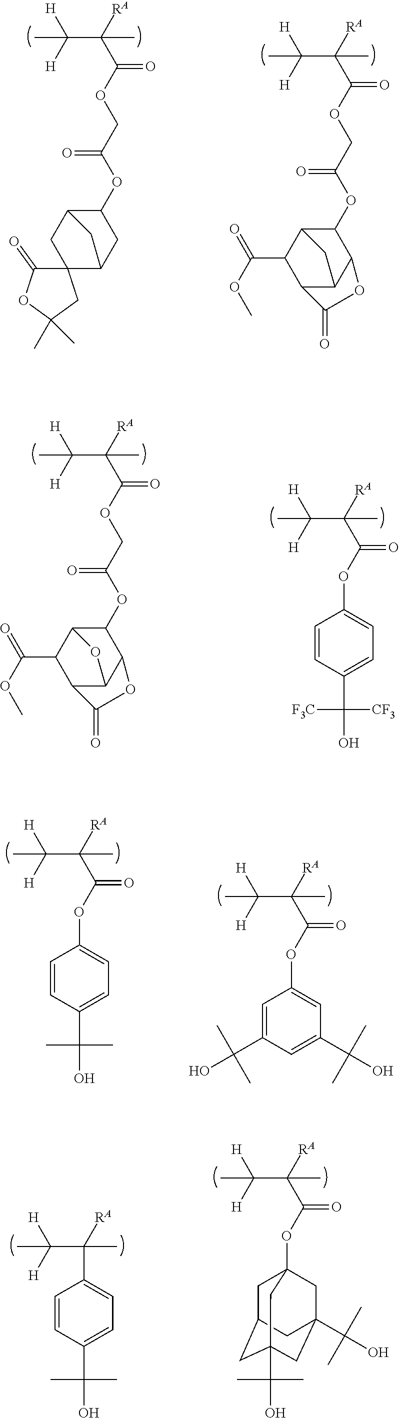

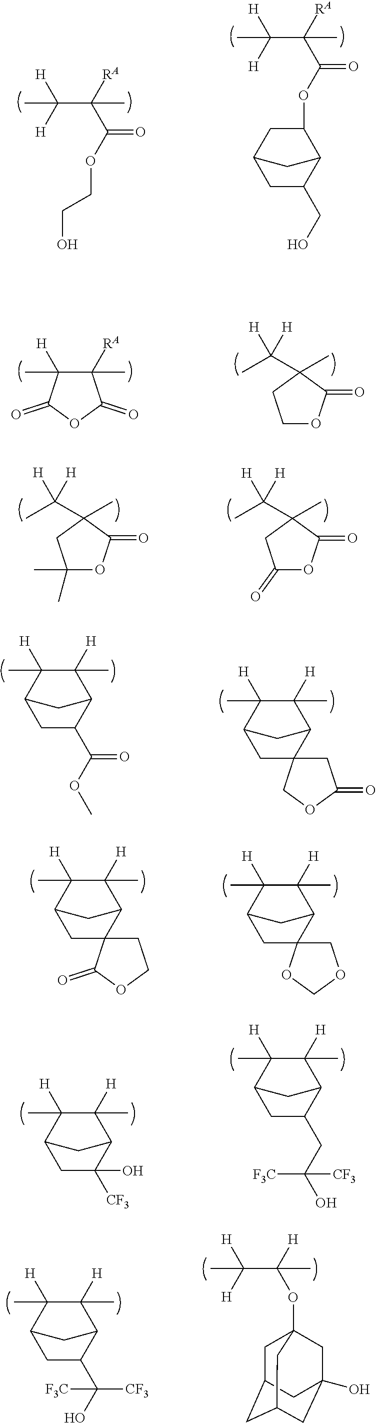

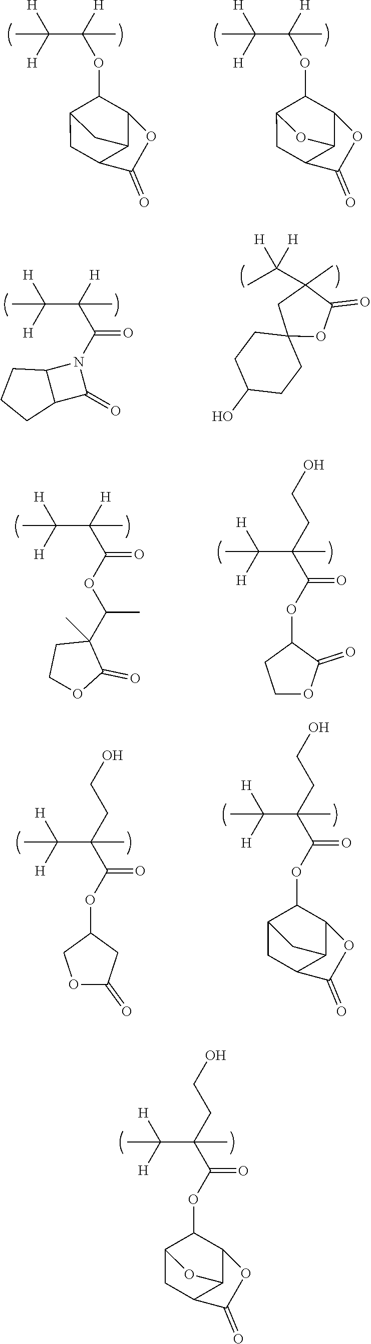

In addition to the foregoing examples, examples of the repeat units (e) include those described in JP-A 2014-225005, paragraphs [0045]-[0053].

Of the foregoing, units having a hydroxy group or lactone ring are preferred as the repeat unit (e), with preferred examples being shown below. Herein R^{A} is as defined above.

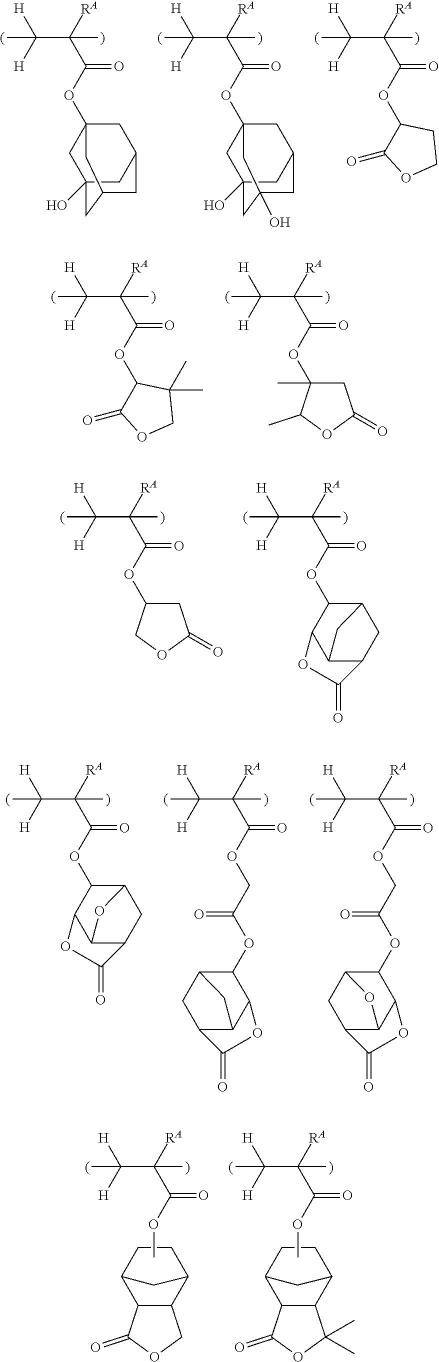

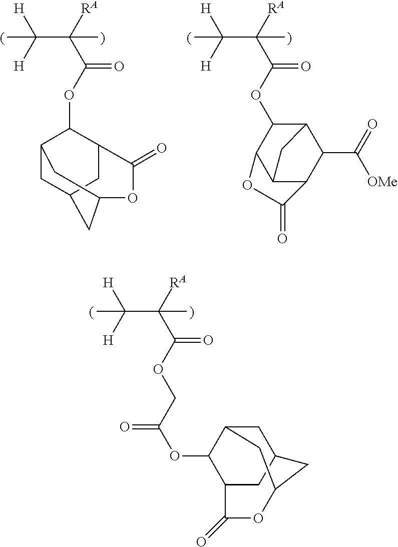

The base polymer may further comprise other repeat units. Typical of the other repeat units are repeat units having an oxirane or oxetane ring. A polymer comprising repeat units having an oxirane or oxetane ring is crosslinked in exposed regions, leading to improvements in retention and etching resistance of a resist film in exposed regions.

The base polymer may further comprise still other repeat units, for example, units derived from substituted acrylates such as methyl crotonate, dimethyl maleate, and dimethyl itaconate, unsaturated carboxylic acids such as maleic acid, fumaric acid, and itaconic acid, cyclic olefins such as norbornene, norbornene derivatives, tetracyclo[6.2.1.1^{3.6}.0²⁷]dodecene derivatives, unsaturated acid anhydrides such as itaconic anhydride, vinyl aromatics such as styrene, tert-butoxystyrene, vinylnaphthalene, acetoxystyrene, and acenaphthylene, and other monomers.

The base polymer should preferably have a Mw of 1,000 to 500,000, more preferably 3,000 to 100,000, and even more preferably 4,000 to 20,000. A Mw within the range eliminates an extreme drop of etching resistance and provides satisfactory resolution due to a difference in dissolution rate before and after exposure. As used herein, Mw is measured versus polystyrene standards by GPC. Also preferably the polymer has a dispersity (Mw/Mn) of 1.20 to 2.50, more preferably 1.30 to 2.00.

The polymer may be synthesized by any method, for example, by using one or more monomers corresponding to the desired repeat units in an organic solvent, adding a radical polymerization initiator, and heating for polymerization. For the polymerization method, reference should be made to U.S. Pat. No. 9,256,127 (JP-A 2015-214634, paragraphs [0134]-[0137]). The acid labile group that has been incorporated in the monomer may be kept as such, or polymerization may be followed by protection or partial protection.

While the base polymer comprises repeat units derived from monomers, the molar fractions of respective units preferably fall in the following range (mol %), but are not limited thereto:
(I) 10 to 70 mol %, more preferably 20 to 65 mol %, even more preferably 30 to 60 mol % of repeat units of at least one type selected from repeat units (a) and (b),
(II) 0 to 90 mol %, more preferably 15 to 80 mol %, even more preferably 30 to 60 mol % of repeat units (c) of at least one type, and optionally,
(III) 0 to 30 mol %, more preferably 0 to 20 mol %, and even more preferably 0 to 15 mol % of repeat units of at least one type selected from repeat units (d1) to (d4), and optionally,
(IV) 0 to 80 mol %, more preferably 0 to 70 mol %, and even more preferably 0 to 50 mol % of repeat units of at least one type selected from repeat units (e) and other repeat units.

The base polymer (A) may be used alone or in a combination of two or more polymers which are different in compositional ratio, Mw and/or Mw/Mn. In addition to the polymer, a hydrogenated product of ring-opening metathesis polymerization (ROMP) polymer may be used. The hydrogenated ROMP polymer is as described in JP-A 2003-066612.

### (B) Photoacid Generator

The resist composition should comprise (B) a photoacid generator, which is sometimes referred to as PAG of addition type, when the base polymer does not contain any of repeat units (d1) to (d4). It is noted that a PAG of addition type may be added even when the base polymer contains repeat units of at least one type selected from repeat units (d1) to (d4).

The PAG of addition type may be any compound capable of generating an acid upon exposure to high-energy radiation. Suitable PAGs include sulfonium salts, iodonium salts, sulfonyldiazomethanes, N-sulfonyloxydicarboxyimides, O-arylsulfonyloximes, and O-alkylsulfonyloximes, which may be used alone or in admixture. Suitable examples are described in JP-A 2007-145797, paragraphs [0102]-[0113], JP-A 2008-111103, paragraphs [0122]-[0142], JP-A 2014-001259, paragraphs [0081]-[0092], JP-A 2012-041320, JP-A 2012-153644, JP-A 2012-106986, and JP-A 2016-018007. The PAGs capable of generating partially fluorinated sulfonic acids described in the foregoing patent documents are preferably used in a resist composition because the strength and diffusion length of the generated acid are appropriate in the ArF lithography.

Preferred as the PAG (B) are sulfonium salts having the formula (3) and iodonium salts having the formula (4).

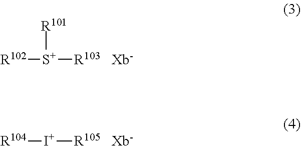

In formulae (3) and (4), R¹⁰¹, R¹⁰², R¹⁰³, R¹⁰⁴ and R¹⁰⁵ are each independently a C₁-C₂₀ hydrocarbyl group which may contain a heteroatom. Examples of the hydrocarbyl group are as exemplified above for R³¹ to R⁴¹ in formulae (d1) to (d4). Any two of R¹⁰¹, R¹⁰² and R¹⁰³ may bond together to form a ring with the sulfur atom to which they are attached, and R¹⁰⁴ and R¹⁰⁵ may bond together to form a ring with the iodine atom to which they are attached. R¹⁰¹ to R¹⁰⁵ are preferably of a structure containing a phenyl group which is bonded to S⁺ or I⁺ in the formula.

The sulfonium cation of the sulfonium salt having formula (3) is described in JP-A 2014-001259, paragraphs [0082]-[0085]. Exemplary sulfonium cations include those described in JP-A 2007-145797, paragraphs [0027]-[0033], JP-A 2010-113209, paragraph [0059], JP-A 2012-041320, JP-A 2012-153644, and JP-A 2012-106986, as well as those exemplified above for the sulfonium cation M⁺ in formula (1) or (2).

Preferred examples of the cation of the sulfonium salt having formula (3) are given below, but not limited thereto.

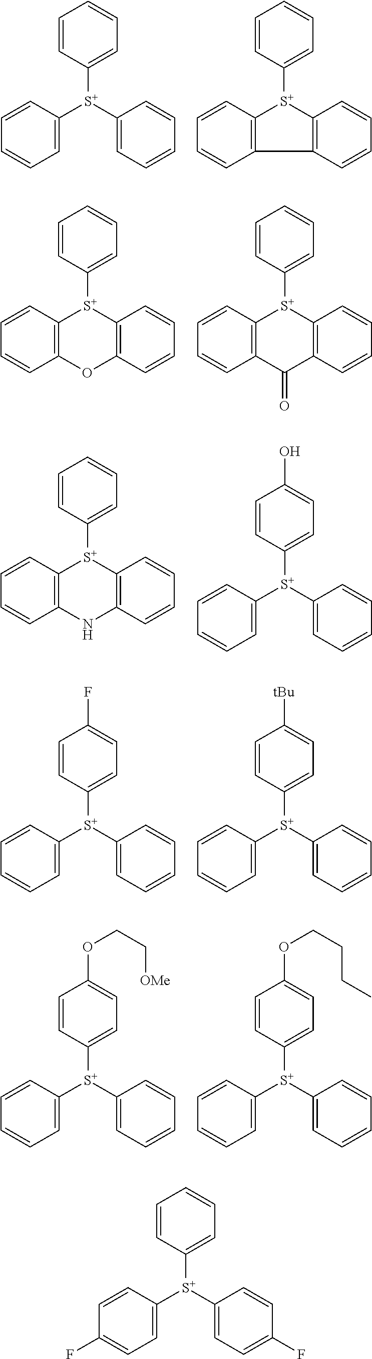

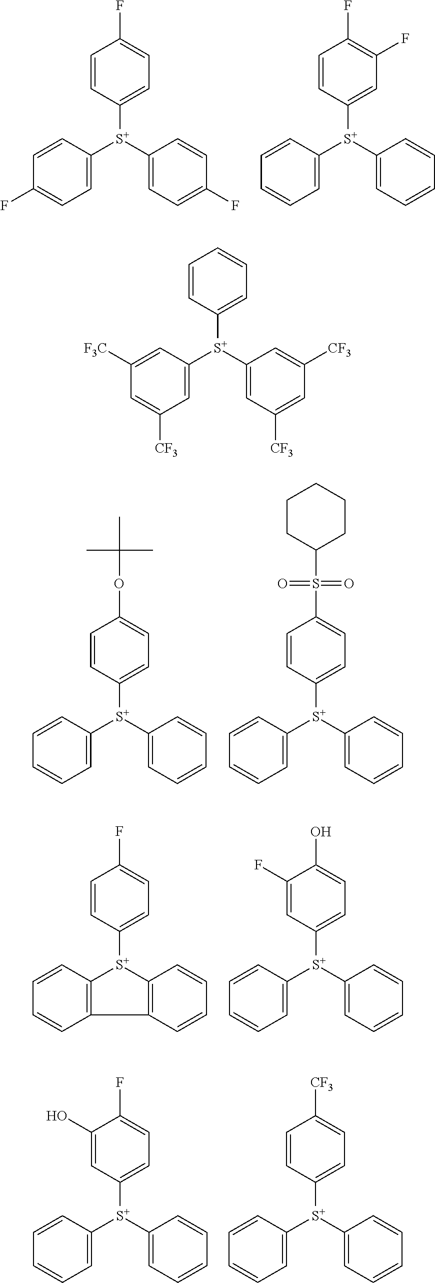

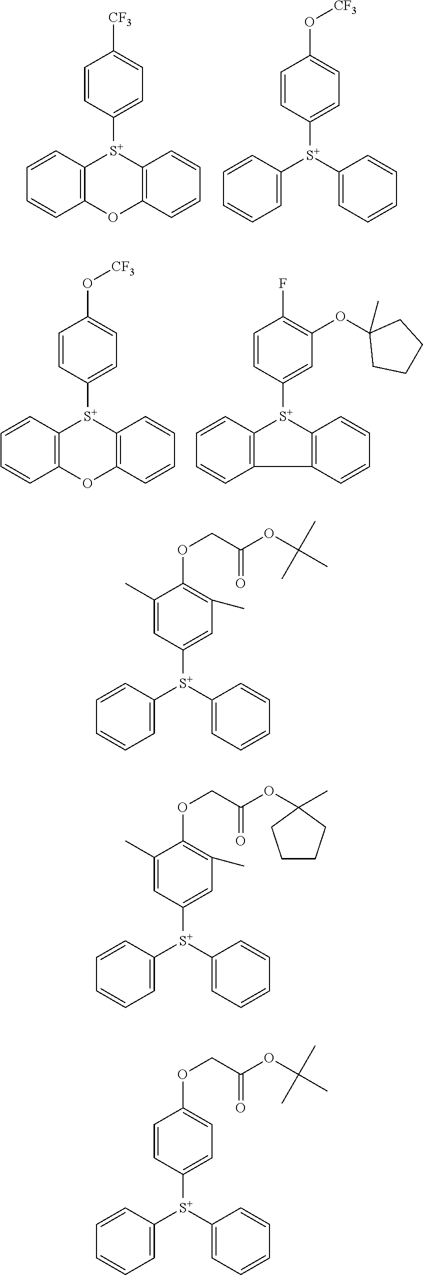

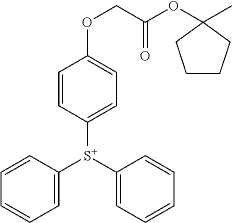 [SMILES: CC1(OC(=O)COc2ccc([S+](c3ccccc3)c3ccccc3)cc2)CCCC1]

Specific examples of the cation of the sulfonium salt having formula (3) include triphenylsulfonium, S-phenyldibenzothiophenium, (4-tert-butylphenyl)diphenylsulfonium, (4-fluorophenyl)diphenylsulfonium, (4-hydroxyphenyl)diphenylsulfonium, and tris(4-fluorophenyl)sulfonium cations.

Examples of the cation of the iodonium salt having formula (4) include those exemplified above for the iodonium cation M⁺ in formula (1) or (2), with diphenyliodonium and di-tert-butylphenyliodonium cations being preferred.

In formulae (3) and (4), Xb⁻ is an anion having the formula (5) or (6).

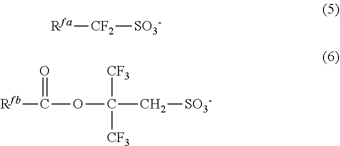

In formulae (5) and (6), R^{fa} is fluorine, a C₁-C₄ perfluoroalkyl group, or a C₁-C₄₀ hydrocarbyl group which may contain a heteroatom. R¹ is a C₁-C₄₀ hydrocarbyl group which may contain a heteroatom. The hydrocarbyl groups represented by R^{fa} and R^{fb} may be saturated or unsaturated and straight, branched or cyclic. Examples thereof are as will be exemplified later for the hydrocarbyl group R¹¹² in formula (5′).

Preferred examples of the anion having formula (5) include trifluoromethanesulfonate and nonafluorobutanesulfonate anions, and anions having the formula (5′).

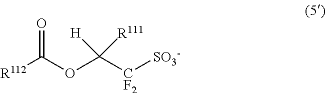

In formula (5′), R¹¹¹ is hydrogen or trifluoromethyl, preferably trifluoromethyl.

R¹¹² is a C₁-C₃₅ hydrocarbyl group which may contain a heteroatom. The hydrocarbyl group may be saturated or unsaturated and straight, branched or cyclic. Examples include alkyl groups such as methyl, ethyl, n-propyl, isopropyl, n-butyl, sec-butyl, tert-butyl, n-pentyl, n-hexyl, n-octyl, 2-ethylhexyl, n-nonyl, n-decyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2.6}]decanyl, adamantyl, adamantylmethyl; alkenyl groups such as vinyl, allyl, propenyl, butenyl, hexenyl; unsaturated alicyclic hydrocarbyl groups such as cyclohexenyl; aryl groups such as phenyl, naphthyl, thienyl, 4-hydroxyphenyl, 4-methoxyphenyl, 3-methoxyphenyl, 2-methoxyphenyl, 4-ethoxyphenyl, 4-tert-butoxyphenyl, 3-tert-butoxyphenyl, 2-methylphenyl, 3-methylphenyl, 4-methylphenyl, 4-ethylphenyl, 4-tert-butylphenyl, 4-n-butylphenyl, 2,4-dimethylphenyl, 2,4,6-triisopropylphenyl, methylnaphthyl, ethylnaphthyl, methoxynaphthyl, ethoxynaphthyl, n-propoxynaphthyl, n-butoxynaphthyl, dimethylnaphthyl, diethylnaphthyl, dimethoxynaphthyl, diethoxynaphthyl; aralkyl groups such as benzyl, 1-phenylethyl, 2-phenylethyl, and combinations thereof. Some or all of the hydrogen atoms in the hydrocarbyl group may be substituted by a moiety containing a heteroatom such as oxygen, sulfur, nitrogen or halogen, and —CH₂— in the hydrocarbyl group may be replaced by a moiety containing a heteroatom such as oxygen, sulfur or nitrogen, so that the group may contain a hydroxy moiety, cyano moiety, carbonyl moiety, ether bond, ester bond, sulfonic ester bond, carbonate bond, lactone ring, carboxylic anhydride (—C(═O)—O—C(═O)—) or haloalkyl moiety.

The anion having formula (5′) is described in JP-A 2007-145797, JP-A 2008-106045, JP-A 2009-007327, JP-A 2009-258695, and JP-A 2012-181306. Examples of the anion having formula (5) include those described in these patent documents and those exemplified above as the anion having formula (d1-1).

The anion having formula (6) is described in JP-A 2010-215608 and JP-A 2014-133723. Examples of the anion having formula (6) include those described in these patent documents and those exemplified above as the anion having formula (d1-2). Notably, the compound having the anion of formula (6) does not have fluorine at the α-position relative to the sulfo group, but two trifluoromethyl groups at the β-position. For this reason, it has a sufficient acidity to sever the acid labile groups in the base polymer. Thus the compound is an effective PAG.

Preferred examples of the anion Xb⁻ are shown below, but not limited thereto. Herein R^{HF} is hydrogen or trifluoromethyl.

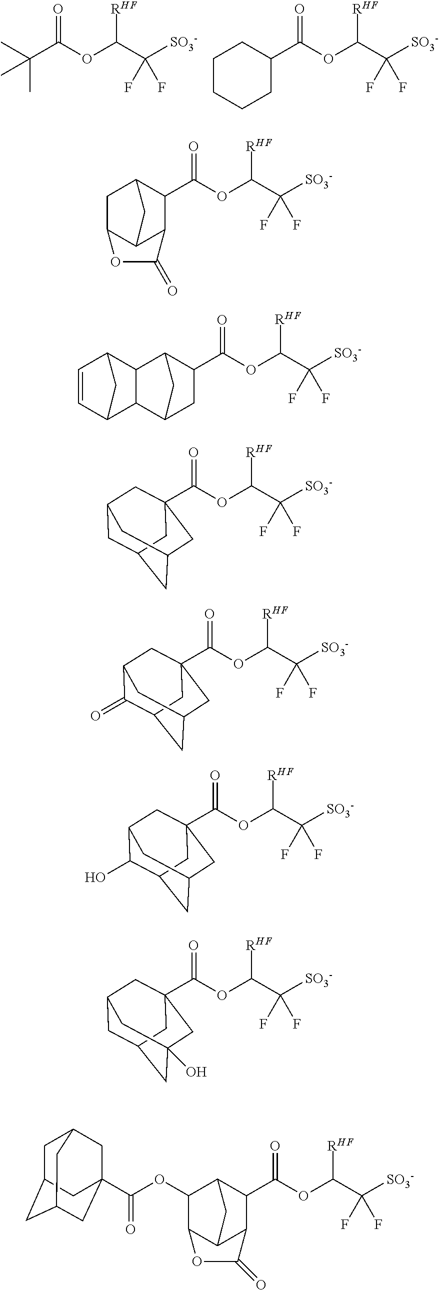

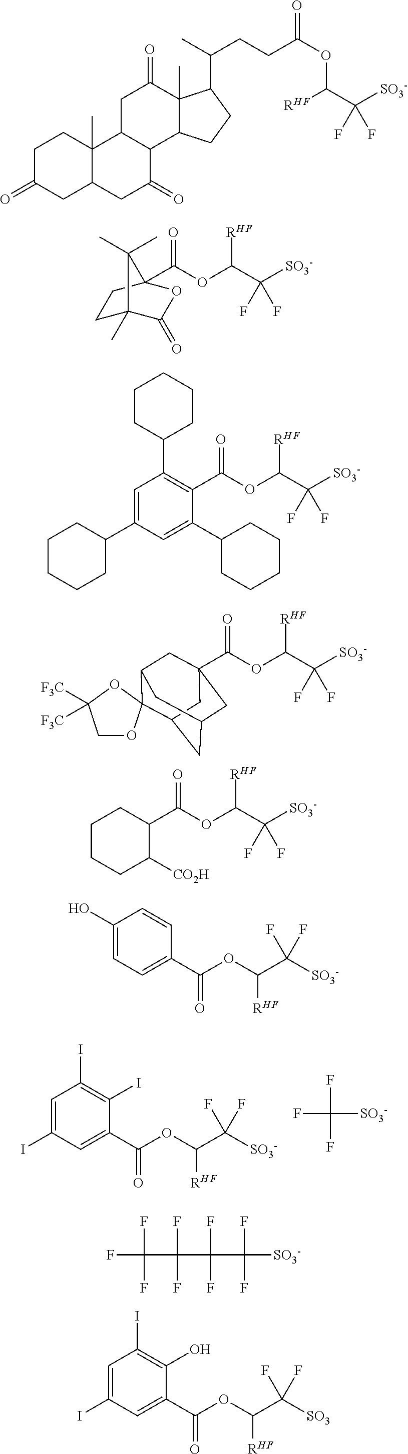

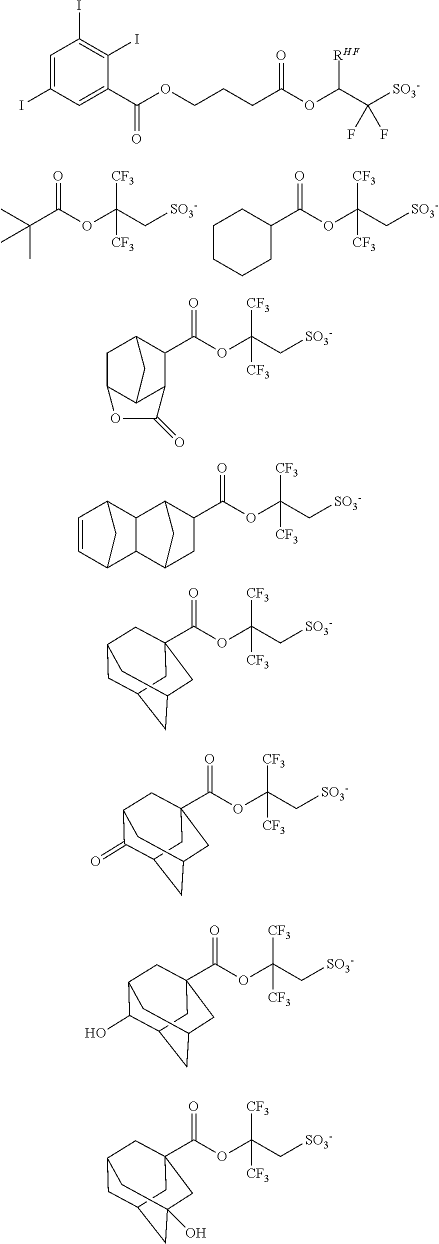

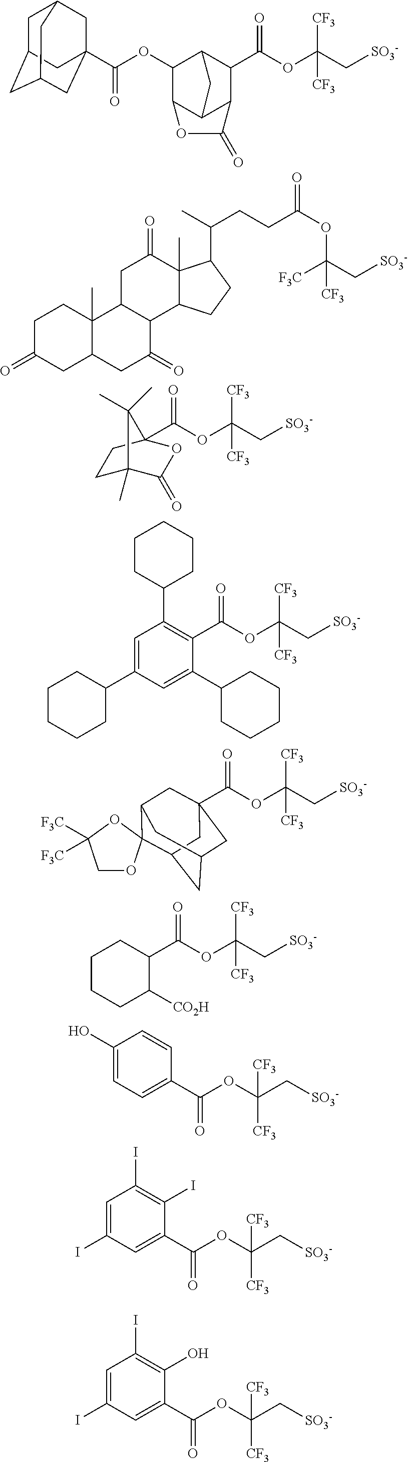

Exemplary structures for the PAG having formula (3) or (4) include arbitrary combinations of cations with anions, both as exemplified above, but are not limited thereto.

Another preferred example of the PAG (B) is a compound having the formula (7).

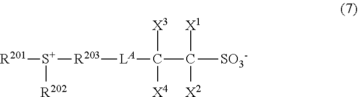

In formula (7), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atom to which they are attached.

The hydrocarbyl groups R²⁰¹ and R²⁰² may be saturated or unsaturated and straight, branched or cyclic. Examples thereof are as exemplified above for the hydrocarbyl group R¹¹² in formula (5′).

The hydrocarbylene group R²⁰³ may be saturated or unsaturated and straight, branched or cyclic. Examples thereof include alkanediyl groups such as methylene, ethylene, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, and tetradecane-1,14-diyl; cyclic saturated hydrocarbylene groups such as cyclopentanediyl, cyclohexanediyl, norbornanediyl, and adamantanediyl; arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, tert-butylnaphthylene, and combinations thereof. In the hydrocarbylene group, some or all hydrogen may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, cyano, carbonyl, ether bond, ester bond, sulfonic ester bond, carbonate bond, lactone ring, sultone ring, carboxylic anhydride (—C(═O)—O—C(═O)—) or haloalkyl moiety.

In formula (7), L^{A} is a single bond, ether bond, ester bond, or a C₁-C₂₀ hydrocarbylene group which may contain a heteroatom. A constituent —CH₂— in the hydrocarbylene group may bond to the carbon atom and/or R²⁰³ in formula (7). Examples of the hydrocarbylene group L^{A} are as exemplified above for R²⁰³.

In formula (7), X¹, X², X³ and X⁴ are each independently hydrogen, fluorine or trifluoromethyl, with at least one thereof being fluorine or trifluoromethyl.

Of the compounds having formula (7), those having formula (7′) are more preferred.

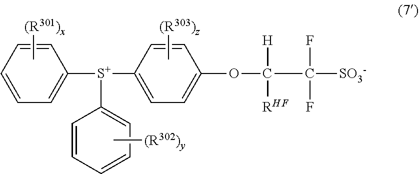

In formula (7′), R^{HF} is hydrogen or trifluoromethyl, preferably trifluoromethyl. R³⁰¹, R³⁰² and R³⁰³ are each independently a C₁-C₂₀ hydrocarbyl group. The hydrocarbyl group may be saturated or unsaturated and straight, branched or cyclic. Examples thereof are as exemplified above for R¹¹² in formula (5′). The subscripts x and y are each independently an integer of 0 to 5, and z is an integer of 0 to 4.

The PAG having formula (7) or (7′) is described in JP-A 2011-016746. Examples thereof include those exemplified for the sulfonium salt in the same patent document and those exemplified for the sulfonium salt in JP-A 2015-214634, paragraphs [0149]-[0150].

Specific examples of the PAG having formula (7) are given below, but not limited thereto. Herein R^{HF} is as defined above.

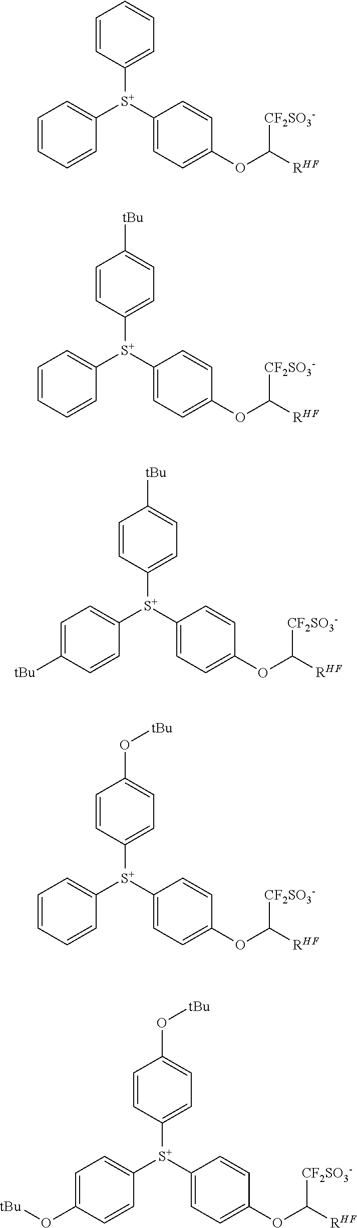

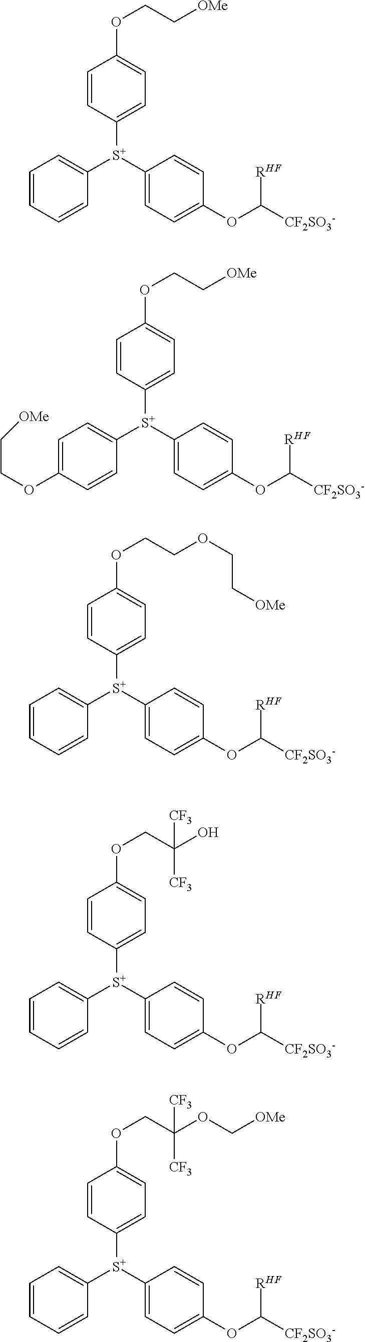

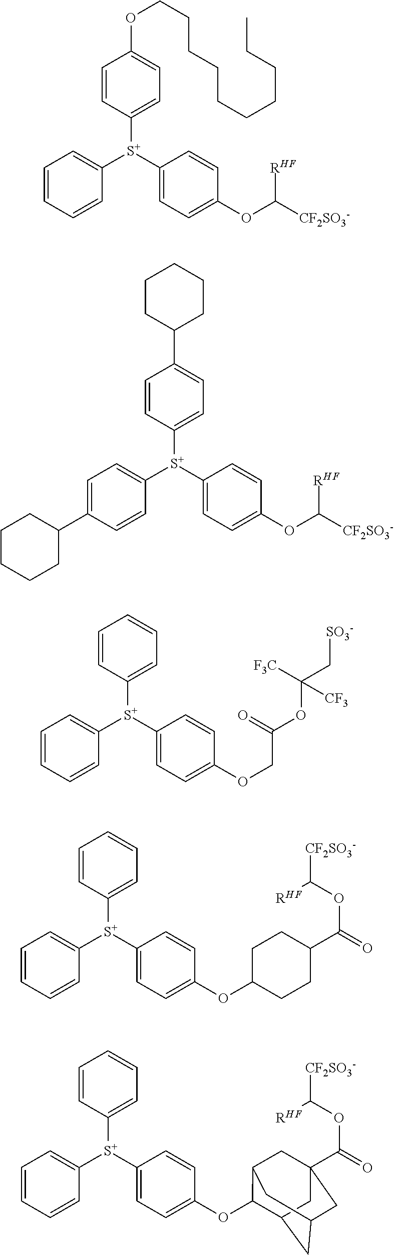

The PAG (B) is preferably added in an amount of 1 to 30 parts by weight, more preferably 2 to 25 parts by weight, even more preferably 4 to 20 parts by weight per 100 parts by weight of the base polymer (A). The PAG in the range eliminates the problems of degradation of resolution and formation of foreign matter after development or during stripping. The PAG may be used alone or in admixture.

### (C) Acid Diffusion Inhibitor

The resist composition further comprises (C) an acid diffusion inhibitor. Component (C) should contain (C-1) the salt compound having formula (1) or (2) as an essential component and may contain (C-2) an acid diffusion inhibitor other than the salt compound having formula (1) or (2). As used herein, the “acid diffusion inhibitor” refers to a compound capable of holding down the diffusion rate when the acid generated by the PAG diffuses in the resist film.

The acid diffusion inhibitor (C-2) is typically selected from amine compounds and onium salts of weak acids such as α-non-fluorinated sulfonic acids and carboxylic acids.

Examples of the amine compound include primary, secondary, and tertiary amine compounds, specifically amine compounds having a hydroxy group, ether bond, ester bond, lactone ring, cyano group or sulfonate bond. Primary and secondary amine compounds protected with a carbamate group are also included. Such protected amine compounds are effective when the resist composition contains a base labile component. Suitable acid diffusion inhibitors include the compounds described in JP-A 2008-111103, paragraphs [0146]-[0164], and JP 3790649 as well as the following compounds, but are not limited thereto.

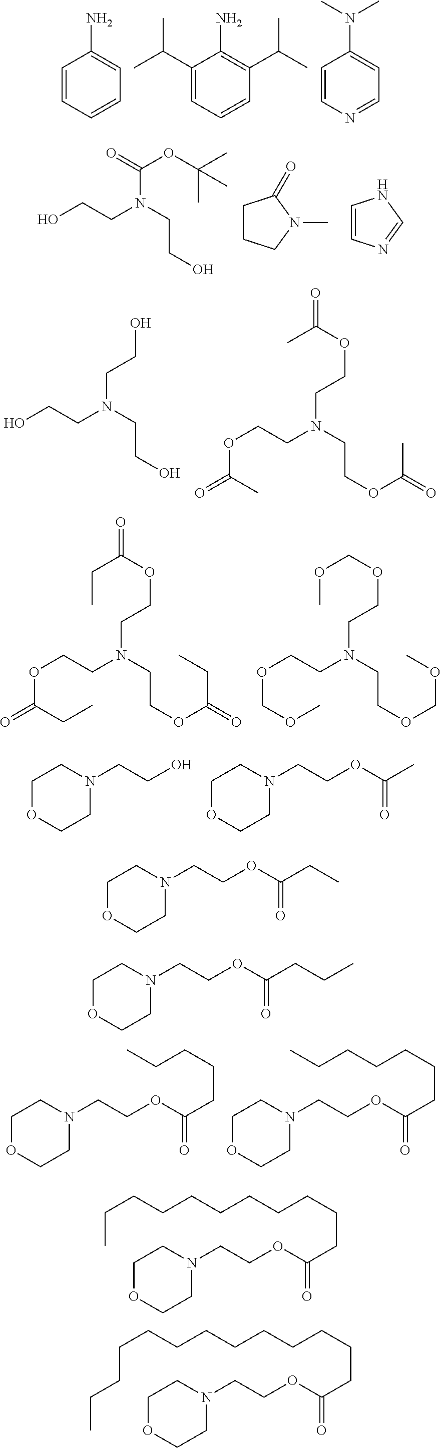

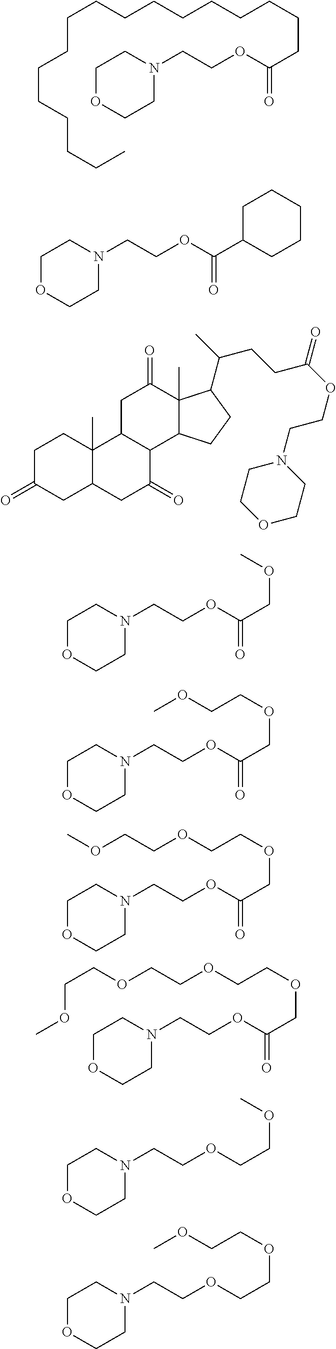

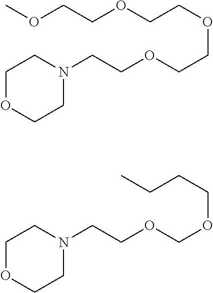 [SMILES: COCCOCCOCCOCCN1CCOCC1 ; CCCCOCOCCN1CCOCC1]

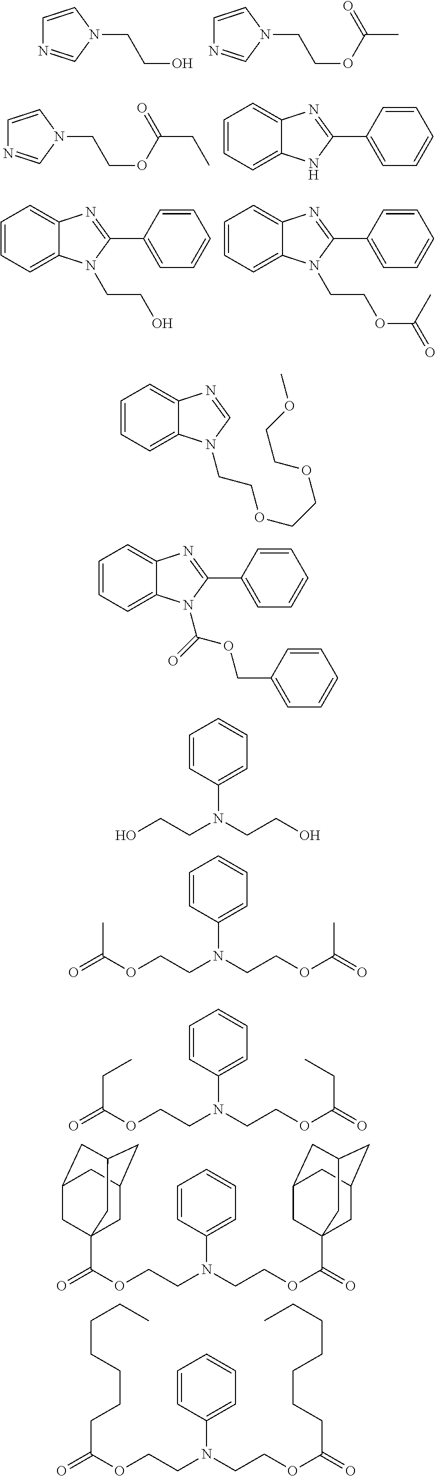

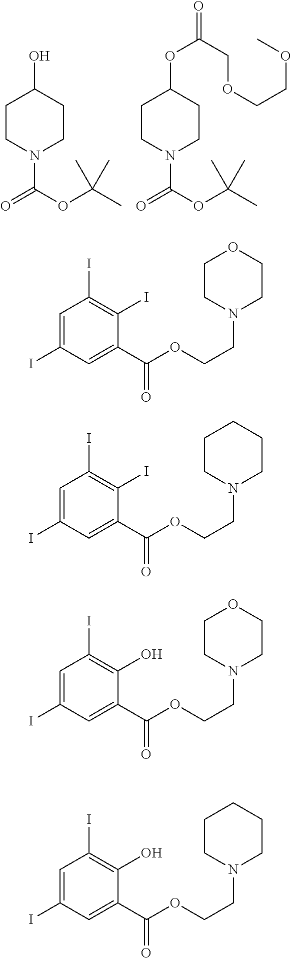

Suitable onium salts of α-non-fluorinated sulfonic acids and carboxylic acids include onium salt compounds having the formulae (8) and (9).

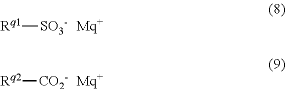

In formula (8), R^{q1} is hydrogen, methoxy, or a C₁-C₄₀ hydrocarbyl group which may contain a heteroatom, exclusive of the group wherein hydrogen bonded to the carbon atom at α-position relative to the sulfo group is substituted by fluorine or fluoroalkyl.

In formula (9), R^{q2} is hydrogen, hydroxy or a C₁-C₄₀ hydrocarbyl group which may contain a heteroatom.

In formulae (8) and (9), Mq⁺ is an onium cation, which is preferably selected from cations having the formulae (10), (11) and (12).

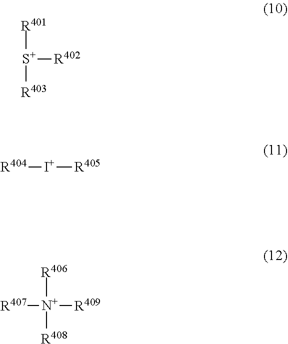

In formulae (10) to (12), R⁴⁰¹ to R⁴⁰⁹ are each independently a C₁-C₄₀ hydrocarbyl group which may contain a heteroatom. A pair of R⁴⁰¹ and R⁴⁰², R⁴⁰4 and R⁴⁰⁵, or R⁴⁰⁶ and R⁴⁰⁷ may bond together to form a ring with the sulfur, iodine or nitrogen atom to which they are attached.

The optionally heteroatom-containing C₁-C₄₀ hydrocarbyl group, represented by R^{q1}, may be saturated or unsaturated and straight, branched or cyclic. Examples thereof include alkyl groups such as methyl, ethyl, n-propyl, isopropyl, n-butyl, sec-butyl, tert-butyl, tert-pentyl, n-pentyl, n-hexyl, n-octyl, 2-ethylhexyl, n-nonyl, and n-decyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2.6}]decanyl, adamantyl, and adamantylmethyl; alkenyl groups such as vinyl, allyl, propenyl, butenyl, and hexenyl; cyclic unsaturated hydrocarbyl groups such as cyclohexenyl; aryl groups such as phenyl and naphthyl; heteroaryl groups such as thienyl; hydroxyphenyl groups such as 4-hydroxyphenyl; alkoxyphenyl groups such as 4-methoxyphenyl, 3-methoxyphenyl, 2-methoxyphenyl, 4-ethoxyphenyl, 4-tert-butoxyphenyl, and 3-tert-butoxyphenyl; alkylphenyl groups such as 2-methylphenyl, 3-methylphenyl, 4-methylphenyl, 4-ethylphenyl, 4-tert-butylphenyl, 4-n-butylphenyl, 2,4-dimethylphenyl and 2,4,6-triisopropylphenyl; alkylnaphthyl groups such as methylnaphthyl and ethylnaphthyl; alkoxynaphthyl groups such as methoxynaphthyl, ethoxynaphthyl, n-propoxynaphthyl and n-butoxynaphthyl; dialkylnaphthyl groups such as dimethylnaphthyl and diethylnaphthyl; dialkoxynaphthyl groups such as dimethoxynaphthyl and diethoxynaphthyl; aralkyl groups such as benzyl, 1-phenylethyl and 2-phenylethyl; aryloxoalkyl groups, typically 2-aryl-2-oxoethyl groups such as 2-phenyl-2-oxoethyl, 2-(1-naphthyl)-2-oxoethyl, 2-(2-naphthyl)-2-oxoethyl; and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, carbonyl moiety, ether bond, ester bond, sulfonic ester bond, carbonate bond, lactone ring, sultone ring, carboxylic anhydride (—C(═O)—O—C(═O)—) or haloalkyl moiety.

The optionally heteroatom-containing C₁-C₄₀ hydrocarbyl group, represented by R^{q2}, may be saturated or unsaturated and straight, branched or cyclic. Examples thereof include those exemplified above for R^{q1} and fluorinated alkyl groups such as trifluoromethyl, trifluoroethyl, 2,2,2-trifluoro-1-methyl-1-hydroxyethyl, and 2,2,2-trifluoro-1-(trifluoromethyl)-1-hydroxyethyl and fluorinated aryl groups such as pentafluorophenyl and 4-trifluoromethylphenyl.

The sulfonic acid onium salt having formula (8) and the carboxylic acid onium salt having formula (9) are described in JP-A 2008-158339 and JP-A 2010-155824. Examples thereof are as exemplified in these patent documents.

Examples of the anion in the sulfonic acid onium salt having formula (8) are shown below, but not limited thereto.

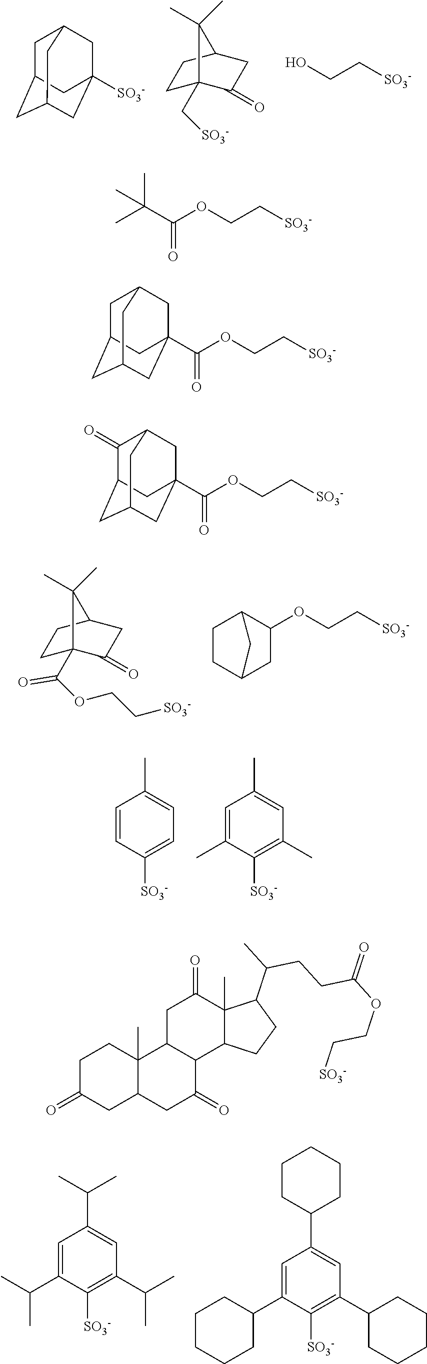

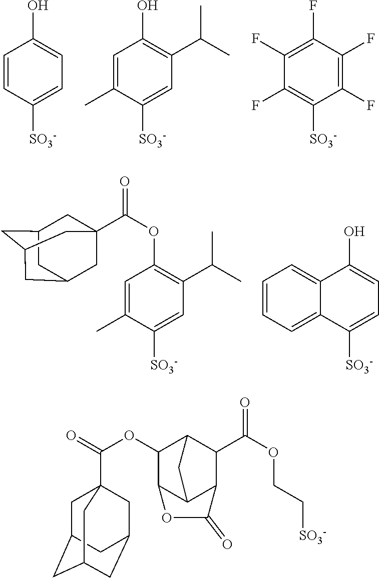

Examples of the anion in the carboxylic acid onium salt having formula (9) are shown below, but not limited thereto.

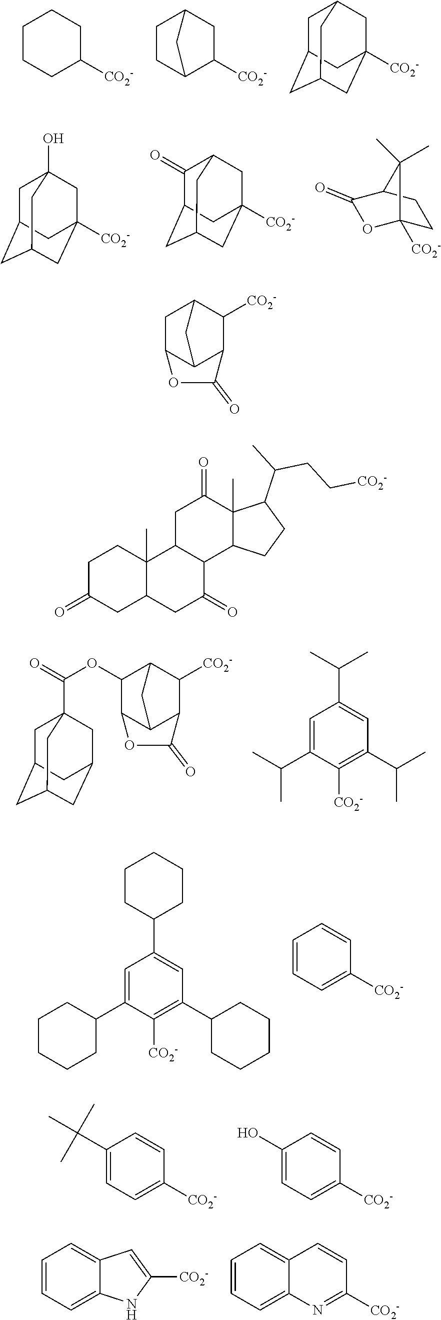

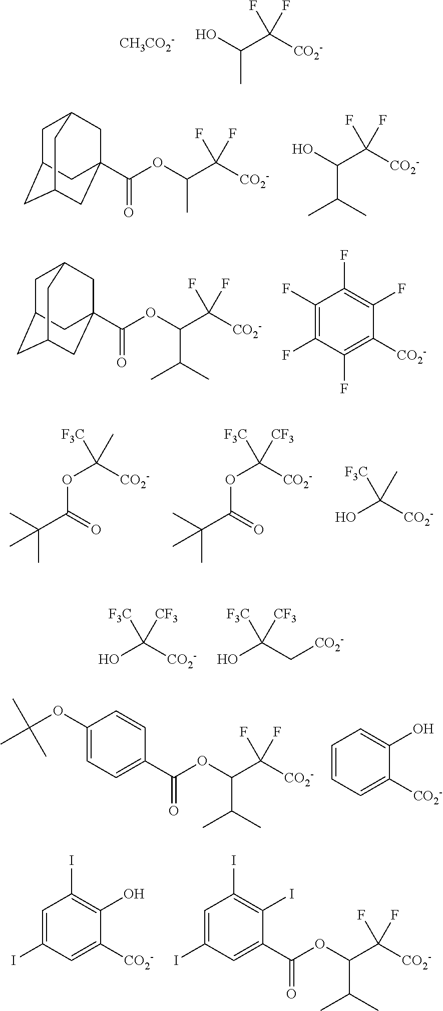

Examples of the cation having formula (10) and the cation having formula (11) are as exemplified above for the sulfonium and iodonium cations in formulae (1) and (2). Examples of the cation having formula (12) include tetramethylammonium, tetraethylammonium, tetrabutylammonium, trimethylbenzyl, and trimethylphenyl cations, but are not limited thereto. Inter alia, more preferred cations are shown below.

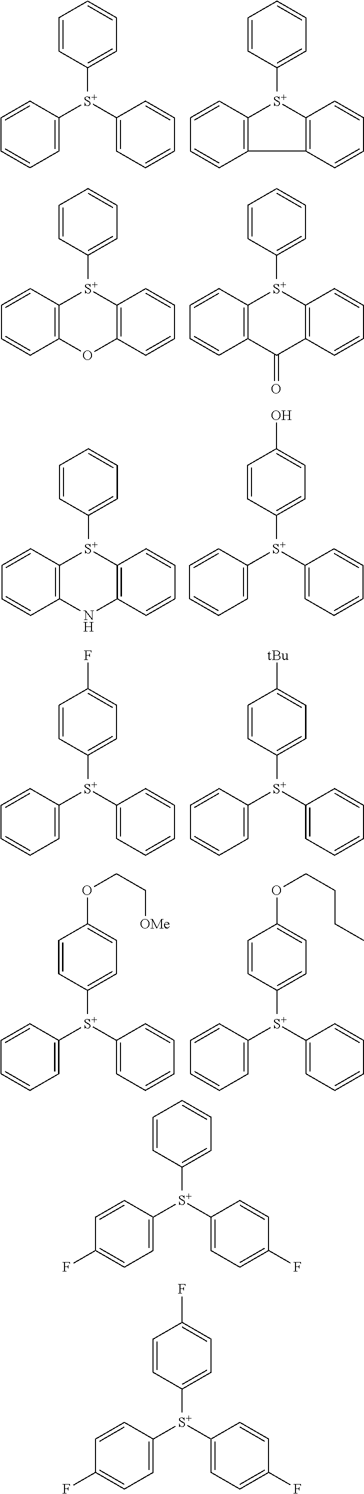

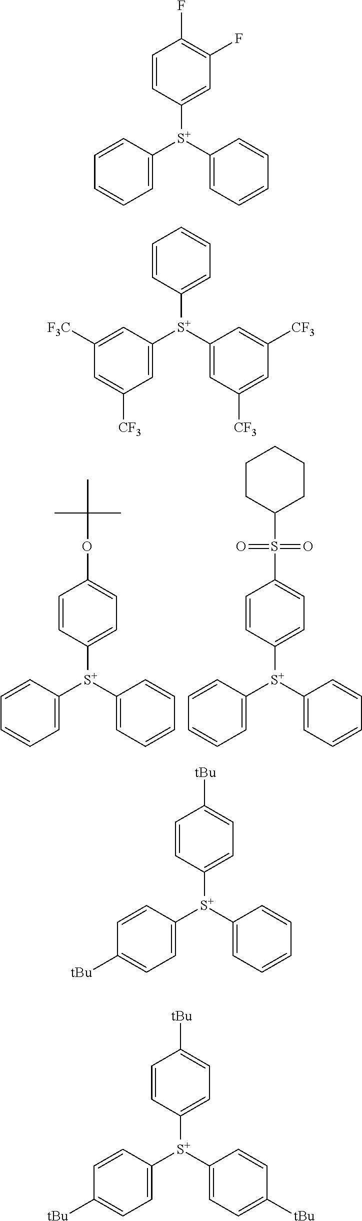 [SMILES: Fc1ccc([S+](c2ccccc2)c2ccccc2)cc1F ; FC(F)(F)c1cc([S+](c2ccccc2)c2cc(C(F)(F)F)cc(C(F)(F)F)c2)cc(C(F)(F)F)c1 ; CC(C)(C)Oc1ccc([S+](c2ccccc2)c2ccccc2)cc1 ; O=S(=O)(c1ccc([S+](c2ccccc2)c2ccccc2)cc1)C1CCCCC1 ; CC(C)(C)c1ccc([S+](c2ccccc2)c2ccc(C(C)(C)C)cc2)cc1 ; CC(C)(C)c1ccc([S+](c2ccc(C(C)(C)C)cc2)c2ccc(C(C)(C)C)cc2)cc1]

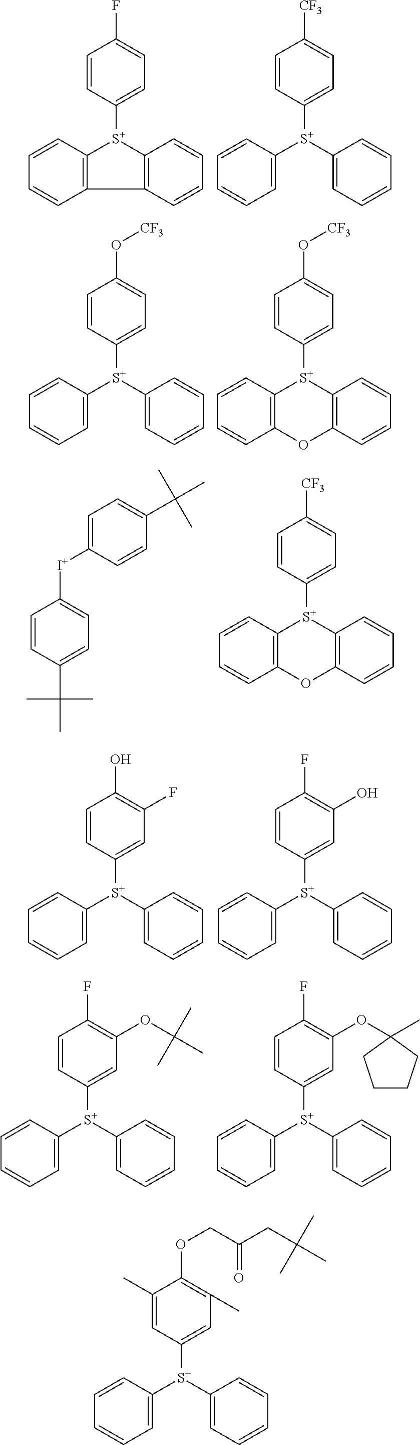

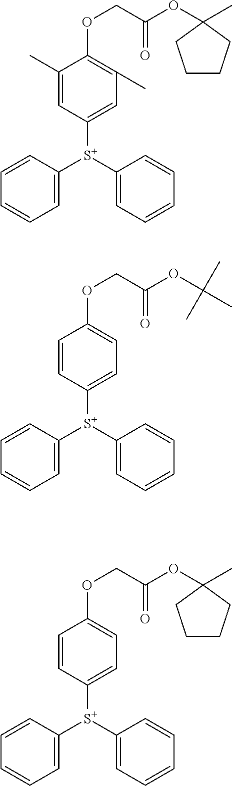 [SMILES: Cc1cc([S+](c2ccccc2)c2ccccc2)cc(C)c1OCC(=O)OC1(C)CCCC1 ; CC(C)(C)OC(=O)COc1ccc([S+](c2ccccc2)c2ccccc2)cc1 ; CC1(OC(=O)COc2ccc([S+](c3ccccc3)c3ccccc3)cc2)CCCC1]

Examples of the sulfonic acid onium salt having formula (8) and the carboxylic acid onium salt having formula (9) include arbitrary combinations of anions with cations, both as exemplified above. These onium salts may be readily synthesized by ion exchange reaction according to any well-known organic chemistry technique. For the ion exchange reaction, reference may be made to JP-A 2007-145797, for example.

The onium salt having formula (8) or (9) functions as an acid diffusion inhibitor in the resist composition because the counter anion of the onium salt is a conjugated base of a weak acid. As used herein, the weak acid indicates an acidity insufficient to deprotect an acid labile group from an acid labile group-containing unit in the base polymer. The onium salt having formula (8) or (9) functions as an acid diffusion inhibitor when used in combination with an onium salt type PAG having a conjugated base of a strong acid (typically α-fluorinated sulfonic acid) as the counter anion. In a system using a mixture of an onium salt capable of generating a strong acid (e.g., α-fluorinated sulfonic acid) and an onium salt capable of generating a weak acid (e.g., non-fluorinated sulfonic acid or carboxylic acid), if the strong acid generated from the PAG upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

Since the onium salt compound having formula (8) or (9) wherein Mq⁺ is a sulfonium cation (10) or iodonium cation (11) is photo-decomposable, the quenching ability is reduced and the concentration of strong acid derived from the PAG is increased in the region with high light intensity. Thus the contrast is improved in the exposed region. As a result, a pattern with improved LWR or CDU can be formed.

In case the acid labile group is an acetal group which is quite sensitive to acid, the acid for eliminating the protective group need not necessarily be an α-fluorinated sulfonic acid, imide acid or methide acid. Sometimes, deprotection reaction can take place even with an α-non-fluorinated sulfonic acid. In this case, an amine compound or carboxylic acid onium salt having formula (9) is preferably used as the acid diffusion inhibitor.

Besides the onium salt, a betaine type compound of weak acid may also be used as the acid diffusion inhibitor. Suitable betaine type compounds are shown below, but not limited thereto.

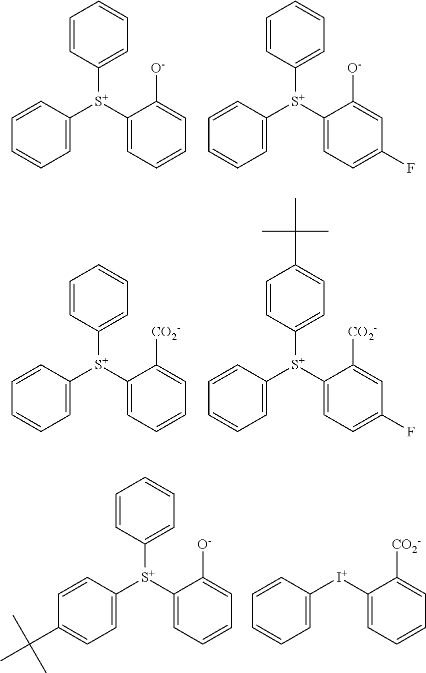

Besides the foregoing compounds, sulfonium or iodonium salts having Cl⁻, Br⁻ or NO₃⁻ as the anion may be used as the acid diffusion inhibitor. Examples include triphenylsulfonium chloride, diphenyliodonium chloride, triphenylsulfonium bromide, and triphenylsulfonium nitrate. Since the conjugate acid corresponding to the anion has a low boiling point, the acid created after quenching of strong acid is readily removed from the resist film during PEB or the like. Due to easy removal of acid from within the resist film, acid diffusion is fully suppressed, resulting in an improvement in contrast.

A photo-decomposable onium salt having a nitrogen-containing substituent may also be used as the acid diffusion inhibitor. The photo-decomposable onium salt functions as an acid diffusion inhibitor in the unexposed region, but as a so-called photo-degradable base in the exposed region because it loses the acid diffusion inhibitory ability due to neutralization thereof with the acid generated by itself. Using a photo-degradable base, the contrast between exposed and unexposed regions can be further enhanced. With respect to the photo-degradable base, reference may be made to JP-A 2009-109595, 2012-046501, and 2013-209360, for example.

Examples of the anion in the photo-degradable oniumn salt are shown below, but not limited thereto. Herein R^{HF} is hydrogen or trifluoromethyl.

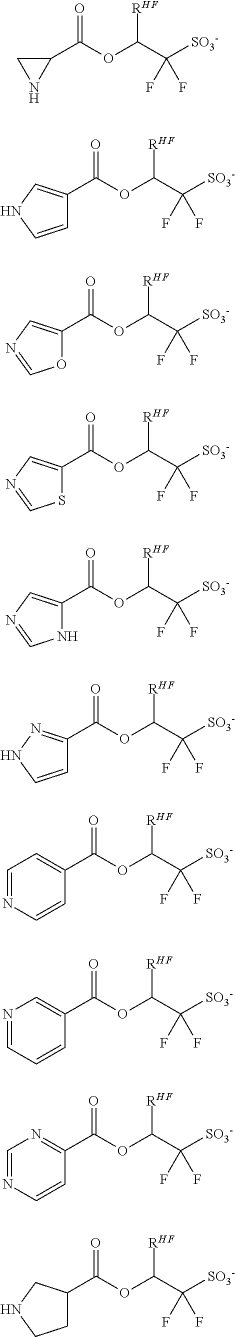

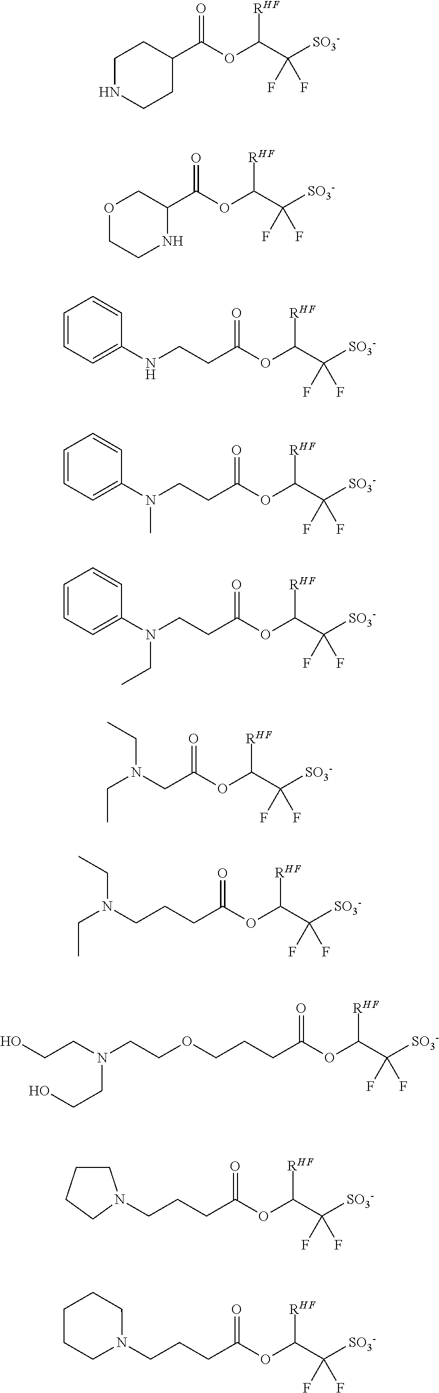

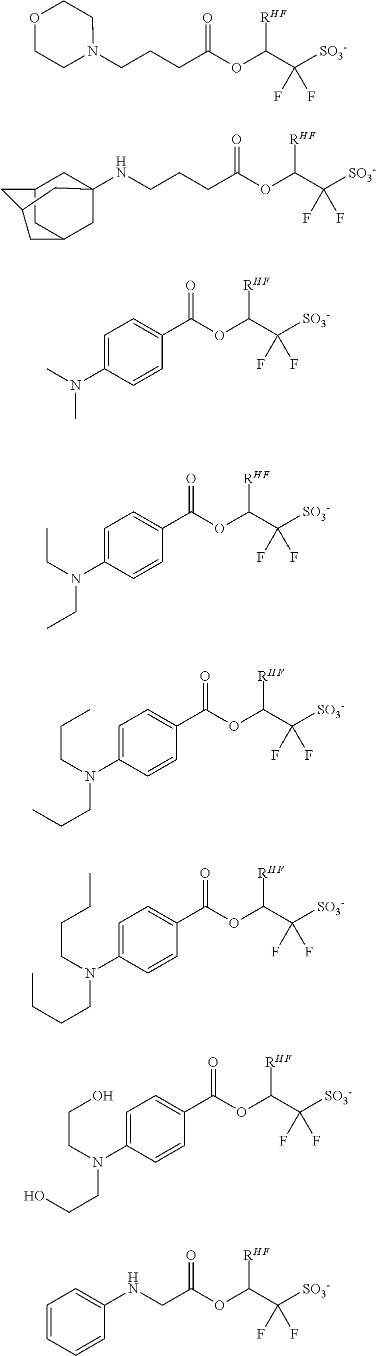

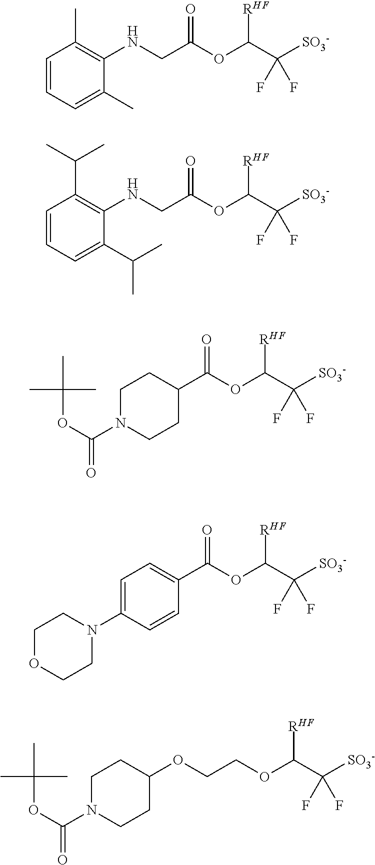

Examples of the cation in the photo-degradable onium salt are as exemplified above for the cation M⁺ in formula (1) or (2). Inter alia, the following cations are preferred, but not limitative.

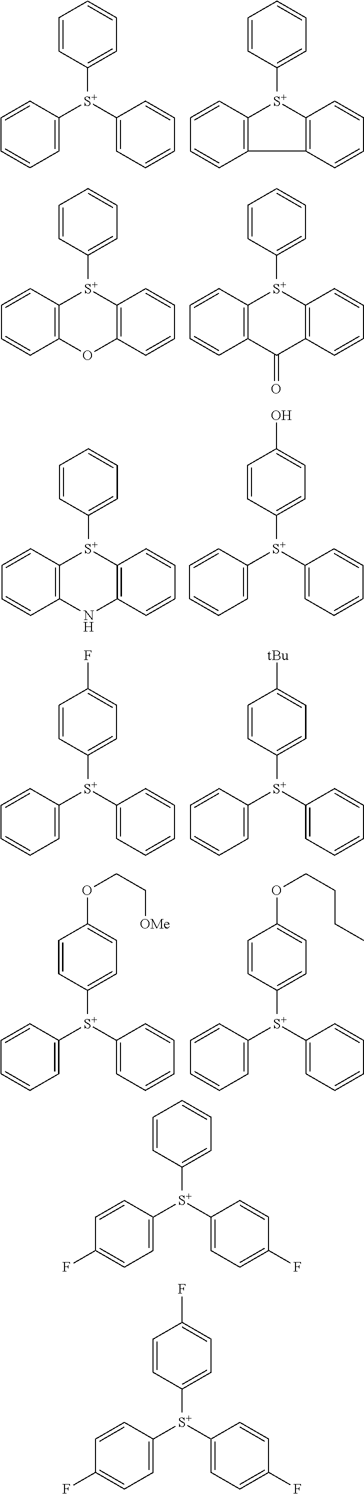

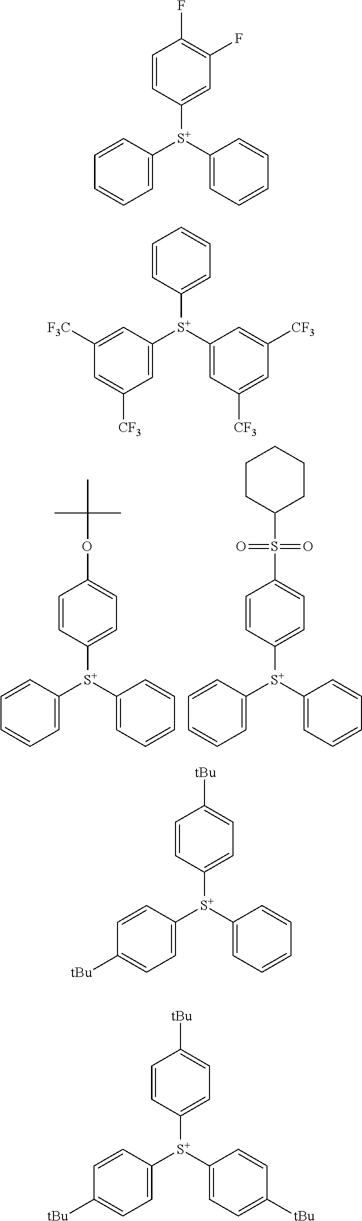 [SMILES: Fc1ccc([S+](c2ccccc2)c2ccccc2)cc1F ; FC(F)(F)c1cc([S+](c2ccccc2)c2cc(C(F)(F)F)cc(C(F)(F)F)c2)cc(C(F)(F)F)c1 ; CC(C)(C)Oc1ccc([S+](c2ccccc2)c2ccccc2)cc1 ; O=S(=O)(c1ccc([S+](c2ccccc2)c2ccccc2)cc1)C1CCCCC1 ; CC(C)(C)c1ccc([S+](c2ccccc2)c2ccc(C(C)(C)C)cc2)cc1 ; CC(C)(C)c1ccc([S+](c2ccc(C(C)(C)C)cc2)c2ccc(C(C)(C)C)cc2)cc1]

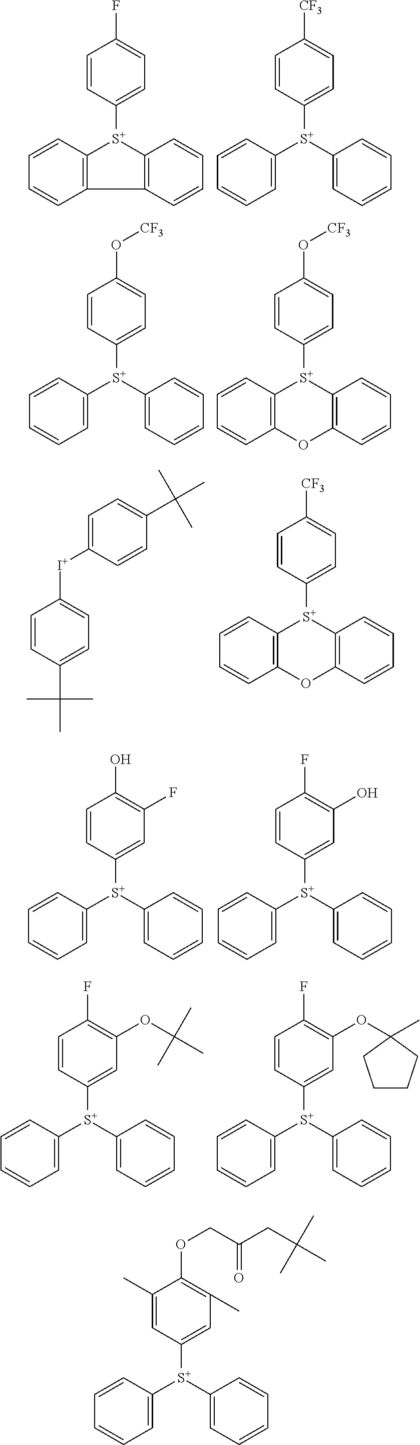

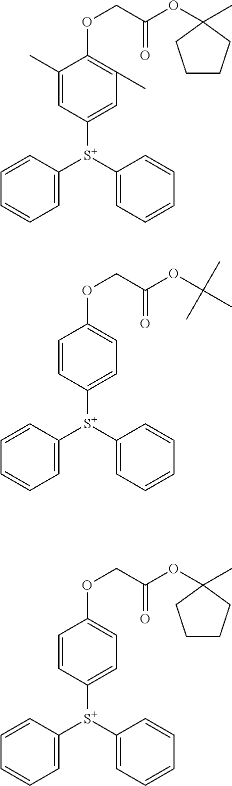 [SMILES: Cc1cc([S+](c2ccccc2)c2ccccc2)cc(C)c1OCC(=O)OC1(C)CCCC1 ; CC(C)(C)OC(=O)COc1ccc([S+](c2ccccc2)c2ccccc2)cc1 ; CC1(OC(=O)COc2ccc([S+](c3ccccc3)c3ccccc3)cc2)CCCC1]

Examples of the photo-decomposable onium salt include arbitrary combinations of cations with anions, both as exemplified above, but are not limited thereto.

Component (C) is preferably used in an amount of 2 to 30 parts by weight, more preferably 2.5 to 20 parts by weight, even more preferably 4 to 15 parts by weight per 100 parts by weight of the base polymer (A). The acid diffusion inhibitor within the range allows for easy adjustment of resist sensitivity, holds down the diffusion rate of acid within the resist film (with improved resolution), suppresses a sensitivity change after exposure, reduces substrate or environment dependency, and improves exposure latitude and pattern profile. Also the addition of the acid diffusion inhibitor is effective for improving substrate adhesion. It is noted that the amount of component (C) is the total amount of the acid diffusion inhibitor in the form of the salt compound having formula (1) or (2) and the acid diffusion inhibitor other than the salt compound having formula (1) or (2). In the acid diffusion inhibitor (C), preferably the onium salt compound having formula (1) or (2) accounts for 50 to 100% by weight. The acid diffusion inhibitor as component (C) may be used alone or in admixture.

### (D) Organic Solvent

The resist composition further comprises (D) an organic solvent. The organic solvent used herein is not particularly limited as long as the foregoing and other components are dissolvable therein. Examples of the organic solvent used herein are described in JP-A 2008-111103, paragraphs [0144]-[0145] (U.S. Pat. No. 7,537,880). Exemplary solvents include ketones such as cyclohexanone (CyHO) and methyl-2-n-pentyl ketone; alcohols such as 3-methoxybutanol, 3-methyl-3-methoxybutanol, 1-methoxy-2-propanol, 1-ethoxy-2-propanol, and diacetone alcohol (DAA); ethers such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, t-butyl acetate, t-butyl propionate, methyl 2-hydroxyisobutyrate, and propylene glycol mono-t-butyl ether acetate; and lactones such as γ-butyrolactone (GBL), which may be used alone or in admixture. Where an acid labile group of acetal form is used, a high boiling alcohol solvent such as diethylene glycol, propylene glycol, glycerol, 1,4-butanediol or 1,3-butanediol may be added to accelerate deprotection reaction of acetal.

Of these organic solvents, preference is given to 1-ethoxy-2-propanol, PGMEA, DAA, CyHO, GBL, ethyl lactate and mixtures thereof because the PAG is highly soluble therein. The preferred solvent system is a mixture of PGMEA as solvent X and at least one of 1-ethoxy-2-propanol, DAA, CyHO, and GBL as solvent Y in a ratio X:Y of from 90:10 to 60:40.

The organic solvent (D) is preferably added in an amount of 100 to 8,000 parts, and more preferably 400 to 6,000 parts by weight per 100 parts by weight of the base polymer (A).

### (E) Surfactant

In addition to the foregoing components, the resist composition may comprise (E) a surfactant which is commonly used for facilitating coating operation.

Component (E) is typically a surfactant which is insoluble or substantially insoluble in water and alkaline developer or a surfactant which is insoluble or substantially insoluble in water and soluble in alkaline developer.

For the surfactant which is insoluble or substantially insoluble in water and alkaline developer, reference should be made to JP-A 2010-215608 and JP-A 2011-016746. Suitable surfactants include FC-4430 (3M), Surflon® S-381, KH-20 and KH-30 (AGC Seimi Chemical Co., Ltd.), Olfine® E1004 (Nisshin Chemical Co., Ltd.), and PolyFox PF-636 (Omnova Solutions, Inc.). Partially fluorinated oxetane ring-opened polymers having the structural formula (surf-1) are also useful.

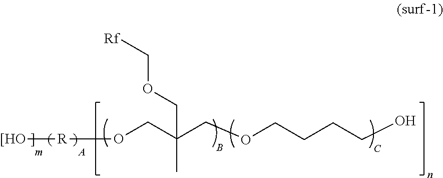

It is provided herein that R, Rf, A, B, C, m, and n are applied to only formula (surf-1), independent of their descriptions other than for the surfactant. R is a di- to tetra-valent C₂-C₅ aliphatic group. Exemplary divalent groups include ethylene, 1,4-butylene, 1,2-propylene, 2,2-dimethyl-1,3-propylene and 1,5-pentylene. Exemplary tri- and tetra-valent groups are shown below.

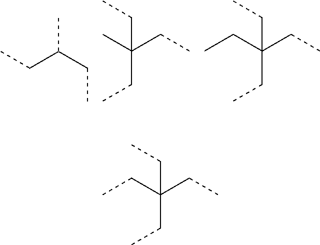

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The letter m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the above structural formula does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist film surface for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from the formulae (13) to (17).

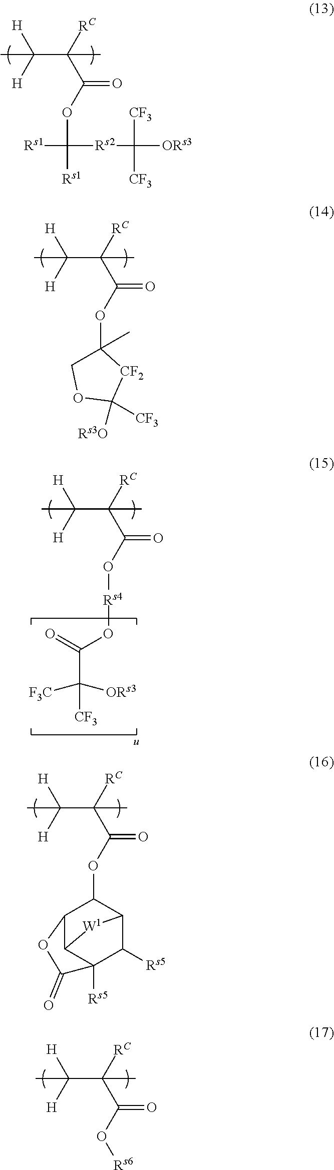

Herein, R^{C} is hydrogen or methyl. W¹ is —CH₂—, —CH₂CH₂— or —O—, or two separate —H. R^{s1} is each independently hydrogen or a C₁-C₁₀ hydrocarbyl group. R^{s2} is a single bond or a C₁-C₅ alkanediyl group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond (—O—) or carbonyl moiety (—C(═O)—) may intervene in a carbon-carbon bond. R^{s4} is a C₁-C₂₀ (u+1)-valent hydrocarbon or fluorinated hydrocarbon group, and u is an integer of 1 to 3. R^{s5} is each independently hydrogen or a group having the formula: —C(═O)—O—R^{s5A} wherein R^{s5A} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The polymeric surfactant may further contain repeat units other than the repeat units having formulae (13) to (17). Typical other repeat units are those derived from methacrylic acid and α-trifluoromethylacrylic acid derivatives. In the polymeric surfactant, the content of the repeat units having formulae (13) to (17) is preferably at least 20 mol %, more preferably at least 60 mol %, most preferably 100 mol % of the overall repeat units.

For the surfactant which is insoluble or substantially insoluble in water and soluble in alkaline developer, reference may be made to JP-A 2008-122932, JP-A 2009-098638, JP-A 2009-191151, JP-A 2009-192784, JP-A 2009-276363, JP-A 2010-107695, JP-A 2010-134012, JP-A 2010-250105, and JP-A 2011-042789.

The amount of component (E) is preferably 0 to 20 parts by weight per 100 parts by weight of the base polymer (A). When added, the amount of component (E) is more preferably 0.001 to 15 parts by weight, even more preferably 0.01 to 10 parts by weight. The surfactant may be used alone or in admixture.

### (F) Other Components

The resist composition may further comprise (F) another component, for example, a compound which is decomposed with an acid to generate another acid (i.e., acid amplifier compound), an organic acid derivative, a fluorinated alcohol, a crosslinker, a compound having a Mw of up to 3,000 which changes its solubility in developer under the action of an acid (i.e., dissolution inhibitor), or an acetylene alcohol. Specifically, the acid amplifier compound is described in JP-A 2009-269953 and JP-A 2010-215608 and preferably used in an amount of 0 to 5 parts, more preferably 0 to 3 parts by weight per 100 parts by weight of the base polymer (A). An extra amount of the acid amplifier compound can make the acid diffusion control difficult and cause degradations to resolution and pattern profile. With respect to the remaining additives, reference should be made to JP-A 2008-122932, paragraphs [0155]-[0182], JP-A 2009-269953 and JP-A 2010-215608.

The resist composition comprising the salt compound having formula (1) or (2) as an acid diffusion inhibitor, when processed by photolithography using high-energy radiation such as KrF excimer laser, ArF excimer laser, EB or EUV, exhibits a high acid diffusion suppressing effect, and forms a pattern at a high contrast and with improved lithography properties such as CDU, LWR and sensitivity.

### Process

A further embodiment of the invention is a pattern forming process using the resist composition defined above. The process includes the steps of applying the resist composition onto a substrate to form a resist film thereon, exposing a selected region of the resist film to high-energy radiation, typically KrF or ArF excimer laser, EB or EUV, and developing the exposed resist film in a developer. Any desired steps may be added to the process if necessary.

The substrate used herein may be a substrate for integrated circuitry fabrication, e.g., Si, SiO₂, SiN, SiON, TiN, WSi, BPSG, SOG, organic antireflective film, etc. or a substrate for mask circuitry fabrication, e.g., Cr, CrO, CrON, MoSi₂, SiO₂, etc.

The resist composition is applied onto a substrate by a suitable coating technique such as spin coating. The coating is prebaked on a hot plate preferably at a temperature of 60 to 180° C. for 10 to 600 seconds, more preferably at 70 to 150° C. for 15 to 300 seconds. The resulting resist film preferably has a thickness of 10 to 2,000 nm.

The resist film is then exposed to high-energy radiation. On use of KrF excimer laser, ArF excimer laser or EUV of wavelength 13.5 nm, the resist film is exposed through a mask having the desired pattern in a dose of preferably 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid between the mask and the resist film may be employed if desired. In the immersion lithography, preferably a liquid having a refractive index of at least 1.0 is held between the resist film and the projection lens. The liquid is typically water, and in this case, a protective film which is insoluble in water may be formed on the resist film.

While the water-insoluble protective film which is used in the immersion lithography serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate at 60 to 150° C. for 1 to 5 minutes, preferably at 80 to 140° C. for 1 to 3 minutes.

The resist film is then developed with a developer in the form of an aqueous base solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle and spray techniques. In this way, a desired resist pattern is formed on the substrate.

With respect to the formation of a positive pattern using an alkaline aqueous solution as the developer, reference may be made to U.S. Pat. No. 8,647,808 (JP-A 2011-231312, paragraphs [0138]-[0146]). With respect to the formation of a negative pattern using an organic solvent as the developer, reference may be made to U.S. Pat. No. 9,256,127 (JP-A 2015-214634, paragraphs [0173]-[0183]).

Any desired step may be added to the pattern forming process. For example, after the resist film is formed, a step of rinsing with pure water (post-soaking) may be introduced to extract the acid generator or the like from the film surface or wash away particles. After exposure, a step of rinsing (post-soaking) may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

Where a hole pattern is formed by negative tone development using organic solvent developer, exposure by double dipole illuminations of X- and Y-direction line patterns provides the highest contrast light. The contrast may be further increased by combining two dipole illuminations of X- and Y-direction line patterns with s-polarized illumination. These pattern forming processes are described in JP-A 2011-221513.

With respect to the developer in the pattern forming process, examples of the aqueous alkaline solution include TMAH aqueous solutions as mentioned above and aqueous alkaline solutions described in JP-A 2015-180748, paragraphs [0148]-[0149], preferably 2 to 3% by weight TMAH aqueous solutions.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. These organic solvents may be used alone or in admixture of two or more.

A hole or trench pattern after development may be shrunk by the thermal flow, RELACS® (resolution enhancement lithography assisted by chemical shrink) or DSA (directed self-assembly) process. A hole pattern is shrunk by coating a shrink agent thereto, and baking such that the shrink agent may undergo crosslinking at the resist surface as a result of the acid catalyst diffusing from the resist layer during bake, and the shrink agent may attach to the sidewall of the hole pattern. The bake is preferably at a temperature of 70 to 180° C., more preferably 80 to 170° C., for a time of 10 to 300 seconds. The extra shrink agent is stripped and the hole pattern is shrunk.

When processed by photolithography, the resist composition comprising the salt compound having formula (1) or (2) as an acid diffusion inhibitor forms a fine size pattern with improved lithography properties such as CDU, LWR and sensitivity.

### EXAMPLES

Examples of the invention are given below by way of illustration and not by way of limitation. The abbreviation “pbw” is parts by weight. For all polymers, Mw and Mn are determined by GPC versus polystyrene standards using tetrahydrofuran (THF) solvent.

### Example 1-1

### Synthesis of Sulfonium Salt Q-1

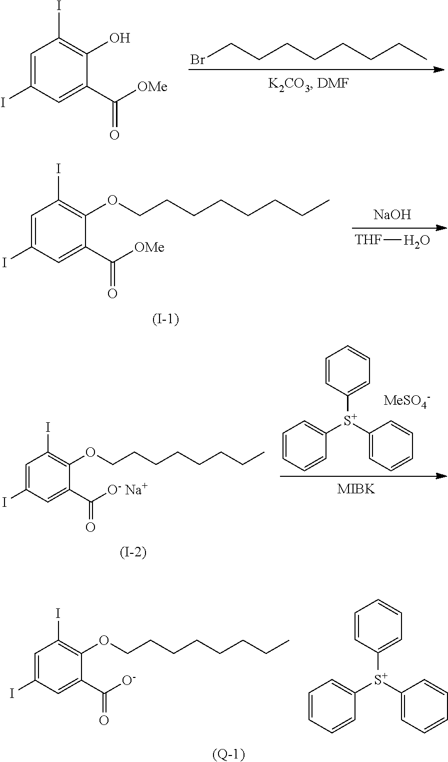

A reactor was charged with 98.2 g of methyl 2,5-diiodobenzoate, 49.3 g of n-octyl bromide, 40.3 g of potassium carbonate, and 392.7 g of N,N-dimethylformamide, which were stirred at 80° C. for 27 hours. After ice cooling, the reaction solution was poured into 800 g of deionized water to quench the reaction, to which 850 g of ethyl acetate was added and stirred. The organic layer was taken out and washed 4 times with 500 g of deionized water. The organic layer was concentrated at 40° C. under reduced pressure, obtaining 128.4 g of an intermediate (I-1) as oily matter (yield 98%).

To a mixture of 128.2 g of intermediate (I-1), 390 g of THF, and 390 g of deionized water, 43.8 g of 25 wt % sodium hydroxide aqueous solution was added dropwise at room temperature, followed by stirring at 40° C. for 24 hours. The reaction solution was concentrated under reduced pressure to remove THF, after which 360 mL of hexane, 95 g of methanol, and 50 g of deionized water were added and stirred. At the end of stirring, the water layer was taken out, to which 360 mL of hexane and 50 g of methanol were added and stirred. The water layer was taken out and washed twice with 300 mL of hexane, obtaining an aqueous solution of an intermediate (I-2). The intermediate was used in the subsequent step without purification.

To the aqueous solution of intermediate (I-2), 108.6 g of triphenylsulfonium methylsulfate, 584 g of methyl isobutyl ketone, and 11.6 g of 1-pentanol were added. After 30 minutes of stirring, the organic layer was taken out. The organic layer was washed 6 times with 100 g of deionized water. To the organic layer, 20 g of methanol and then 7.5 g of active carbon were added. The mixture was stirred overnight. After removal of the active carbon, the solution was washed once with 200 g of 0.7 wt % oxalic acid water, 2 times with 200 g of deionized water, once with 200 g of 1 wt % ammonia water, and 8 times with 200 g of deionized water. The organic layer was concentrated at 50° C. under reduced pressure, obtaining 165.4 g of the target sulfonium salt Q-1 as oily matter (two-step yield 84%).

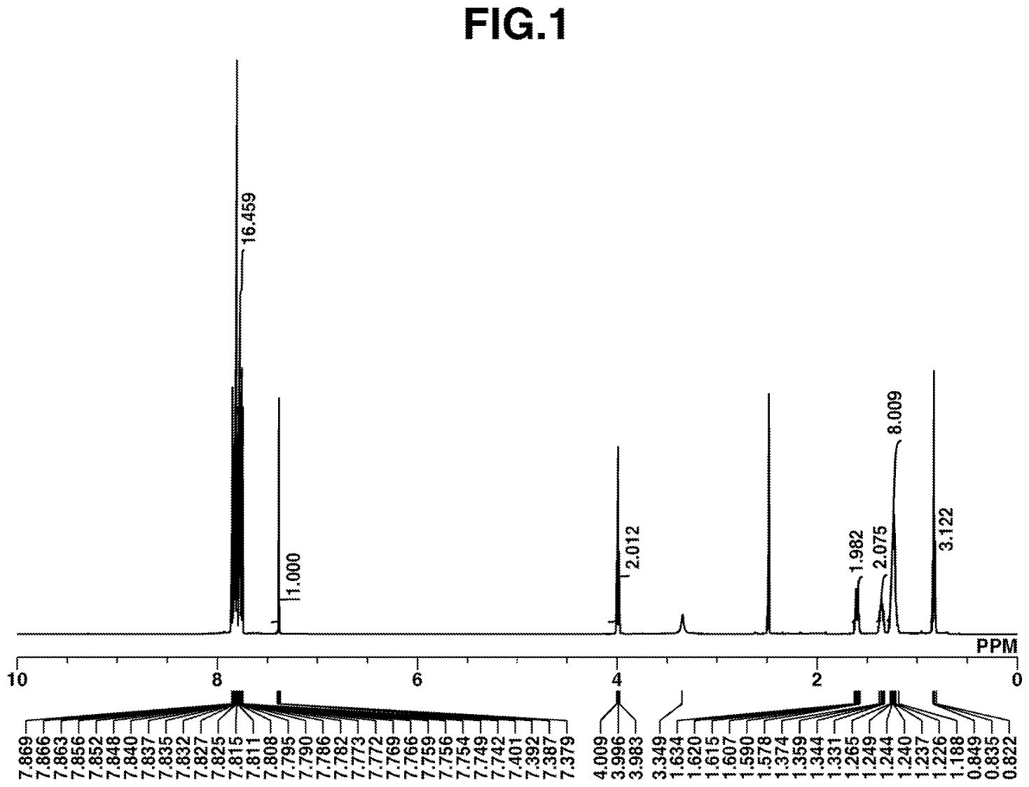

The sulfonium salt Q-1 was analyzed by spectroscopy. The ¹H-NMR spectrum (500 MHz, DMSO-d₆) is shown in FIG. 1. The data of IR spectroscopy and time-of-flight mass spectrometry are shown below.

### IR (D-ATR):

v=3388, 3057, 2952, 2925, 2853, 1708, 1602, 1531, 1476, 1446, 1426, 1378, 1346, 1233, 1102, 1089, 1065, 1022, 996, 861, 749, 696, 685, 503 cm⁻¹

### TOFMS; MALDI

Positive M⁺ 263.1 (corresponding to C₁₈H₁₅S⁺)

Negative M⁻ 500.9 (corresponding to C₁₅H₁₉I₂O₃⁻)

### Example 1-2

### Synthesis of Sulfonium Salt Q-2

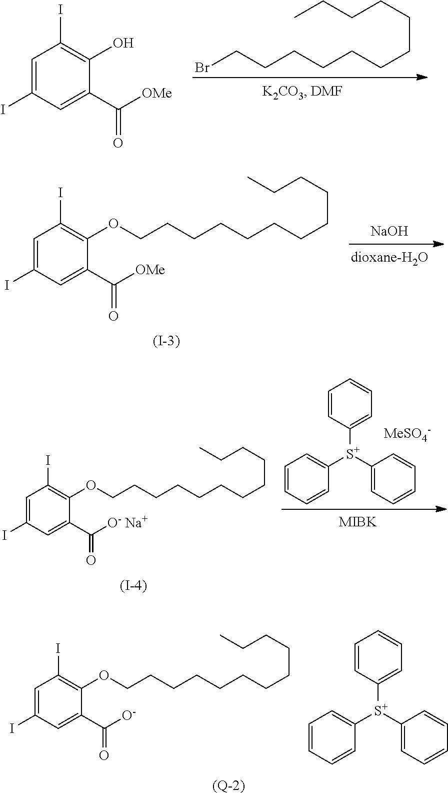

A reactor was charged with 8.1 g of methyl 3,5-diiodosalicylate, 6.5 g of 1-bromododecane, 4.4 g of potassium carbonate, and 60 g of N,N-dimethylformamide, which were stirred at 80° C. for 16 hours. After ice cooling, the reaction solution was poured into 120 g of hydrochloric acid to quench the reaction, to which 100 g of methylene chloride was added and stirred. The organic layer was taken out and washed 4 times with 60 g of deionized water. The organic layer was concentrated at 50° C. under reduced pressure, obtaining 12.5 g of an intermediate (1-3) as oily matter (yield 97%).

To a mixture of 12.5 g of intermediate (1-3), 50 g of dioxane, and 5 g of deionized water, 3.8 g of 25 wt % sodium hydroxide aqueous solution was added dropwise at room temperature, followed by stirring at 40° C. for 17.5 hours. The reaction solution was concentrated under reduced pressure to remove dioxane and water, after which 60 g of diisopropyl ether was added and stirred for 20 minutes. The solid precipitate was filtered, obtaining 7.2 g of an intermediate (1-4) in wet crystal form. The intermediate was used in the subsequent step without vacuum drying.

A mixture of 7.2 g of wet crystal intermediate (1-4), 7.5 g of triphenylsulfonium methylsulfate, 60 g of methyl isobutyl ketone, 5 g of methanol, 20 g of 1-pentanol and 20 g of deionized water was stirred for 50 minutes. The organic layer was taken out. The organic layer was washed 7 times with 20 g of deionized water. The organic layer was concentrated at 50° C. under reduced pressure, obtaining 15.7 g of the target sulfonium salt Q-2 as oily matter (two-step yield 76%).

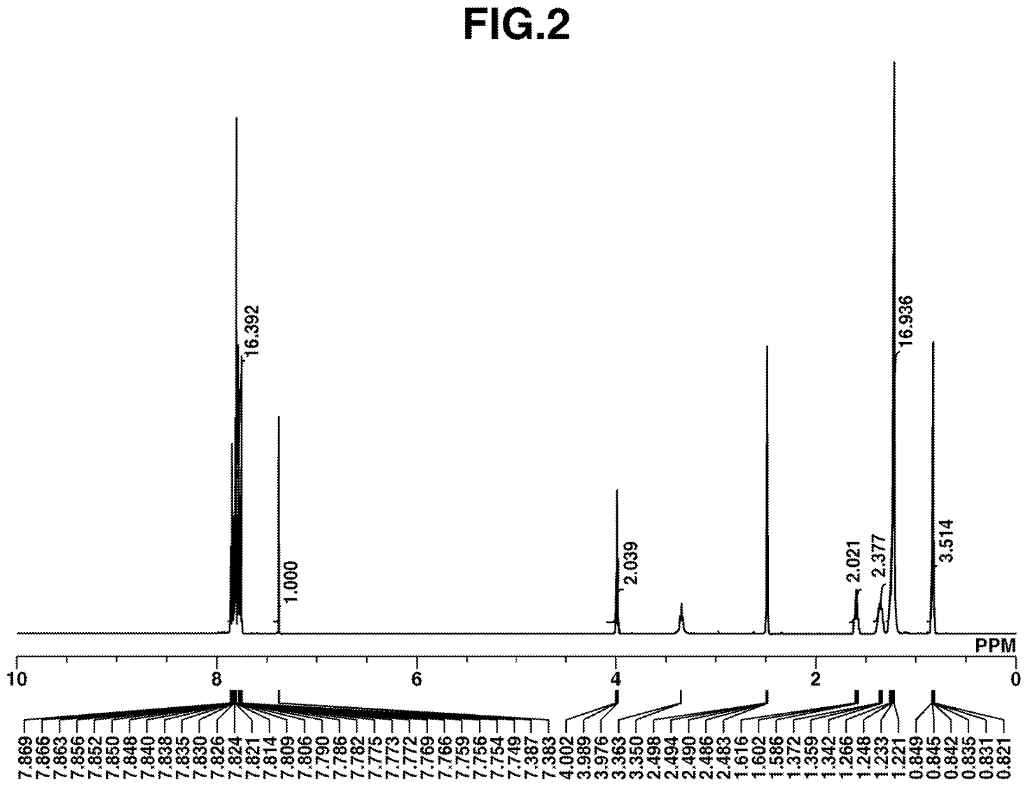

The sulfonium salt Q-2 was analyzed by spectroscopy. The ¹H-NMR spectrum (500 MHz, DMSO-d₆) is shown in FIG. 2. The data of IR and TOFMS are shown below.

### IR (D-ATR):

v=3367, 3056, 2923, 2852, 1603, 1531, 1476, 1446, 1427, 1378, 1345, 1233, 1102, 1088, 1065, 966, 860, 749, 696, 685, 503 cm⁻¹

### TOFMS; MALDI

Positive M⁺ 263.1 (corresponding to C₁₈H₁₅S⁺)

Negative M⁻ 557.0 (corresponding to C₁₉H₂₇I₂O₃⁻)

### Example 1-3

### Synthesis of Sulfonium Salt Q-3

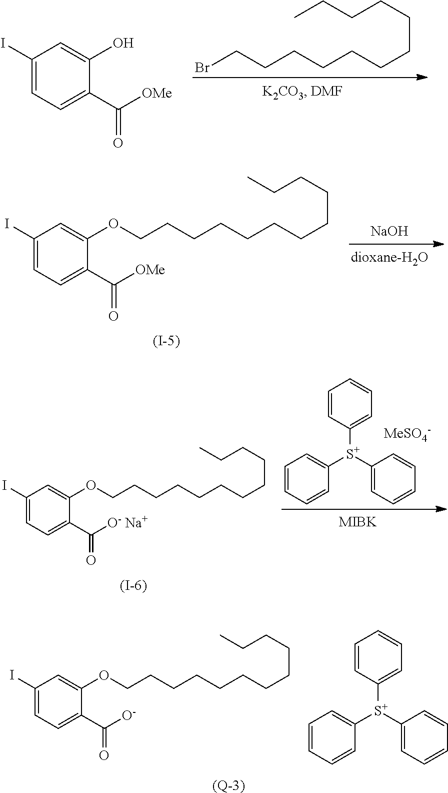

A reactor was charged with 5.0 g of methyl 4-iodosalicylate, 5.8 g of 1-bromododecane, 4.0 g of potassium carbonate, and 50 g of N,N-dimethylformamide, which were stirred at 80° C. for 16 hours. After ice cooling, 100 g of hydrochloric acid was added to the reaction solution to quench the reaction, to which 80 g of methylene chloride was added and stirred. The organic layer was taken out and washed 4 times with 50 g of deionized water. The organic layer was concentrated at 50° C. under reduced pressure, obtaining 9.0 g of an intermediate (I-5) as oily matter (yield 98%).

To a mixture of 8.9 g of intermediate (I-5), 36 g of dioxane, and 4.5 g of deionized water, 3.4 g of 25 wt % sodium hydroxide aqueous solution was added dropwise at room temperature, followed by stirring at 40° C. for 17 hours. The reaction solution was concentrated under reduced pressure to remove dioxane and water, after which 50 g of diisopropyl ether was added and stirred for 20 minutes. The solid precipitate was filtered, obtaining 7.3 g of an intermediate (I-6) in wet crystal form. The intermediate was used in the subsequent step without vacuum drying.

A mixture of 7.3 g of wet crystal intermediate (I-6), 6.7 g of triphenylsulfonium methylsulfate, 50 g of methyl isobutyl ketone, 5 g of methanol, 15 g of 1-pentanol and 20 g of deionized water was stirred for 70 minutes. The organic layer was taken out. The organic layer was washed 4 times with 20 g of deionized water. The organic layer was concentrated at 50° C. under reduced pressure, obtaining 12.3 g of the target sulfonium salt Q-3 as oily matter (two-step yield 81%).

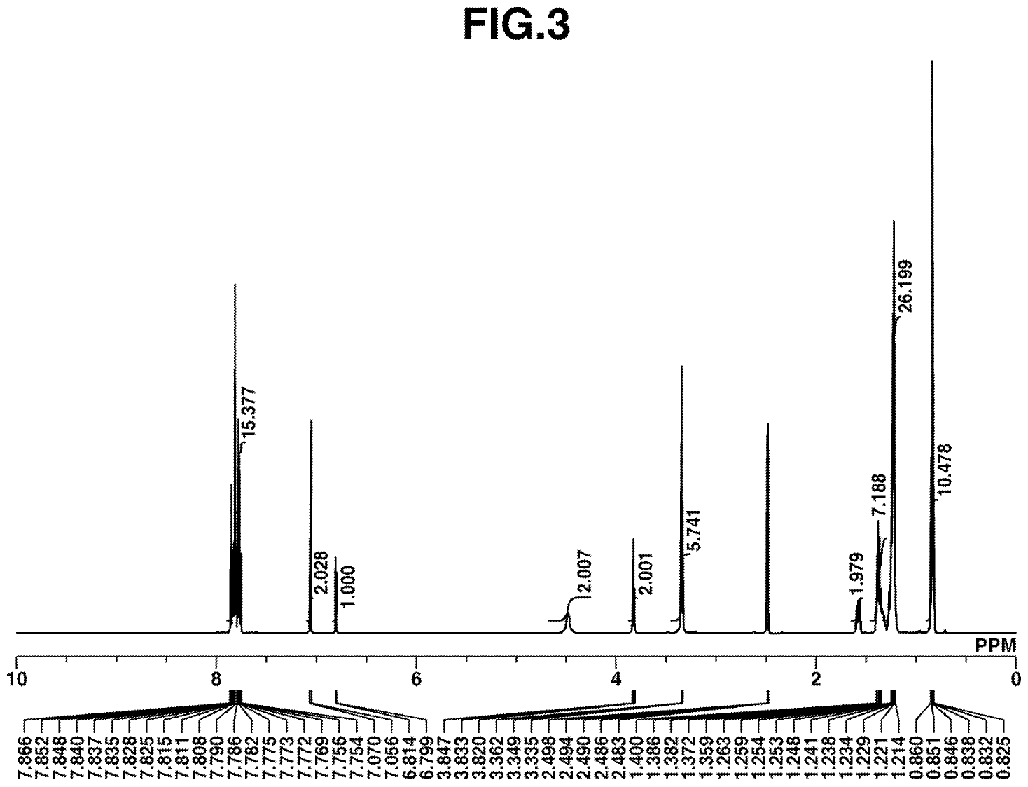

The sulfonium salt Q-3 was analyzed by spectroscopy. The ¹H-NMR spectrum (500 MHz, DMSO-d₆) is shown in FIG. 3. The data of TOFMS are shown below.

### TOFMS; MALDI

Positive M⁺ 263.1 (corresponding to C₁₈H₁₅S⁺)

Negative M⁻ 431.1 (corresponding to C₁₉H₂₈IO₃⁻)

### Examples 1-4 to 1-18

The following sulfonium salts Q-4 to Q-18 were synthesized as in the foregoing Examples.

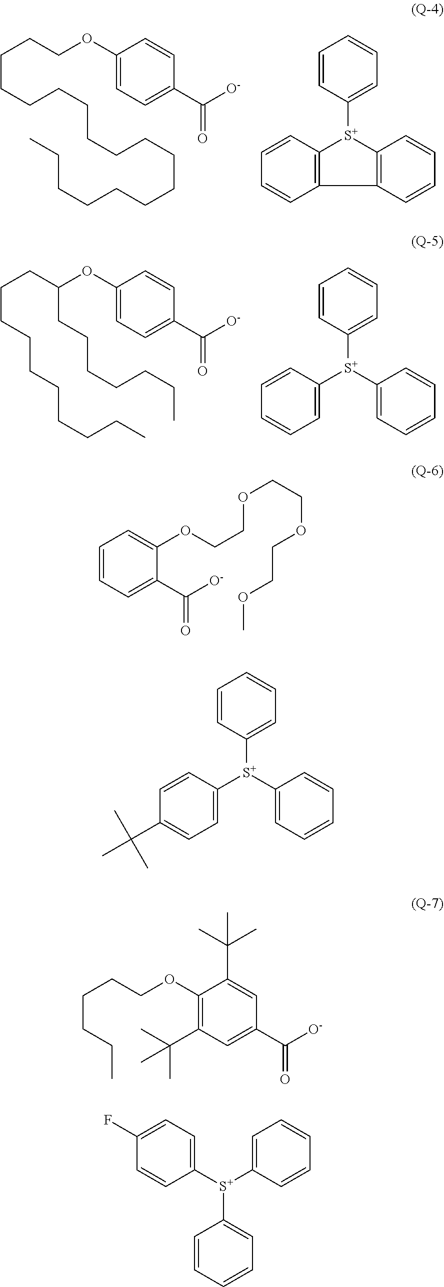

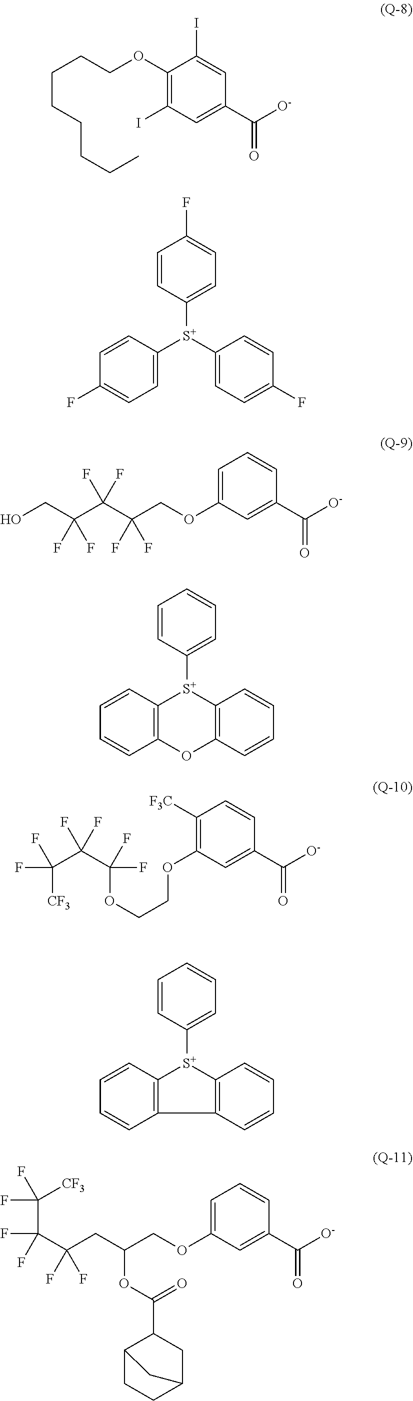

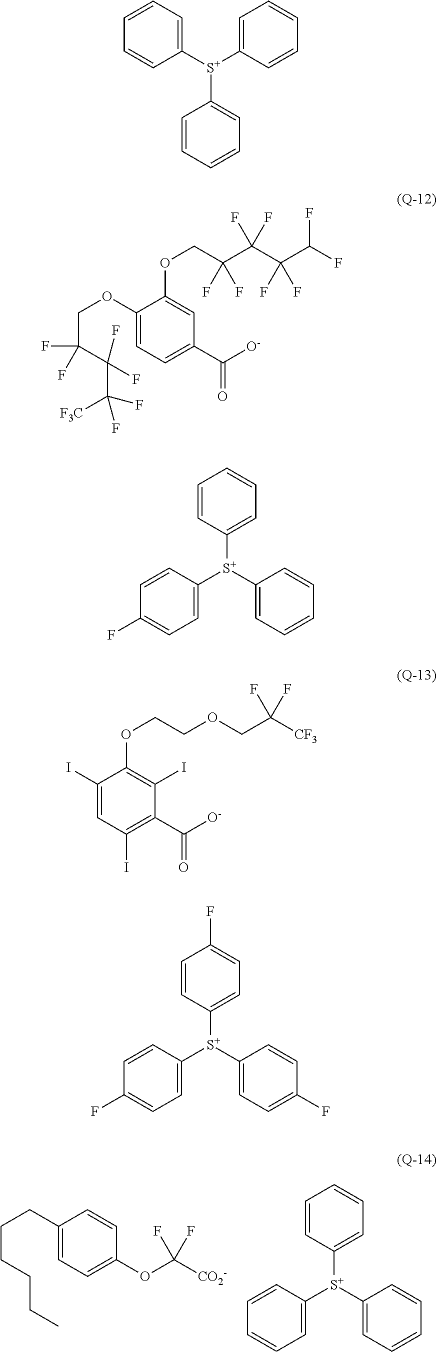

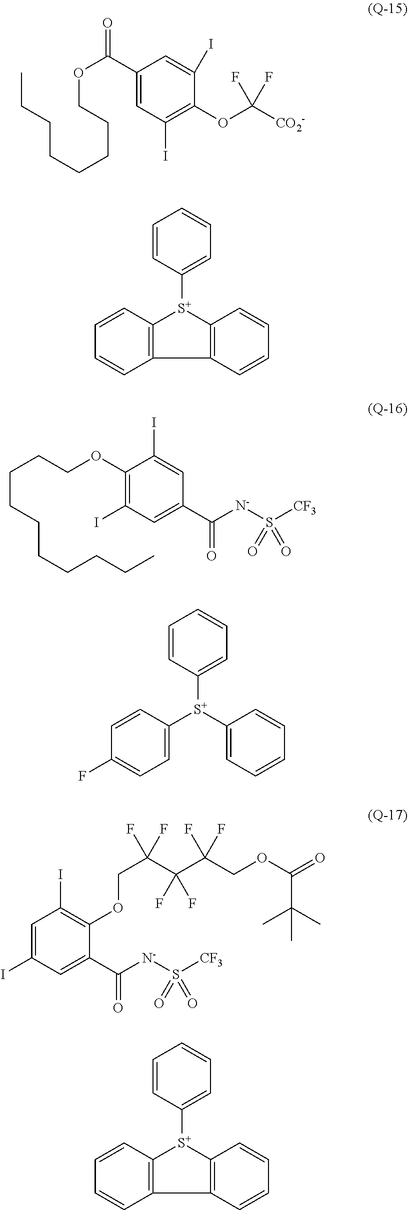

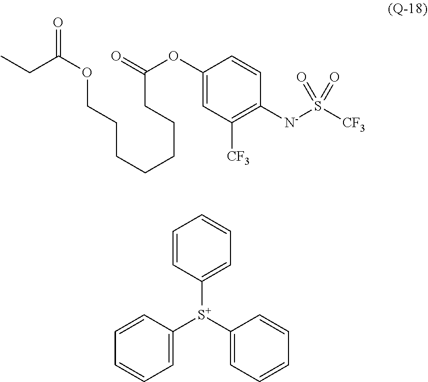 [SMILES: CCC(=O)OCCCCCCCC(=O)Oc1ccc([N-]S(=O)(=O)C(F)(F)F)c(C(F)(F)F)c1 ; c1ccc([S+](c2ccccc2)c2ccccc2)cc1]

### Synthesis Example 1

### Synthesis of Polymer P-1

In nitrogen atmosphere, 2.8 g of triphenylsulfonium 1,1,3,3,3-pentafluoro-2-methacryloyloxypropane-1-sulfonate, 12.3 g of 3-ethyl-3-exo-tetracyclo[4.4.0.1^{2.5}.1^{7.10}]dodecyl methacrylate, 9.0 g of 4,8-dioxatricyclo[4.2.1.0^{3.7}]nonan-5-on-2-yl methacrylate, 2.4 g of 3-hydroxy-1-adamantyl methacrylate, and 0.9 g of dimethyl 2,2′-azobis(isobutyrate) were dissolved in 72.8 g of methyl ethyl ketone (MEK). While 20.7 g of MEK was stirred in nitrogen atmosphere at 80° C., the solution was added dropwise to the MEK over 4 hours. After the completion of dropwise addition, the polymerization solution was continuously stirred for 2 hours while maintaining the temperature of 80° C. After the polymerization solution was cooled to room temperature, it was added dropwise to 400 g of hexane. The solid precipitate was collected by filtration, washed twice with a mixture of 45 g of MEK and 195 g of hexane, and vacuum dried at 50° C. for 20 hours, obtaining Polymer P-1 in white powder form (amount 25.2 g, yield 95%). Polymer P-1 had a Mw of 8,200 and a dispersity Mw/Mn of 1.63.

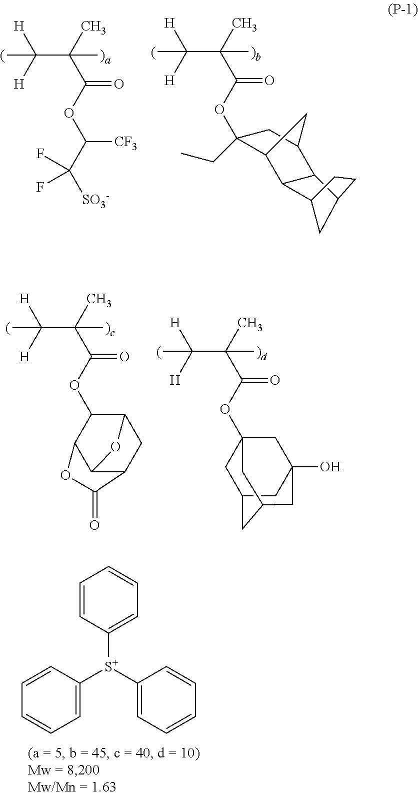

### Synthesis Examples 2 to 4

### Synthesis of Polymers P-2 to P-4

Polymers P-2 to P-4 were synthesized by the same procedure as in Synthesis Example 1 aside from changing the type and amount of monomers.

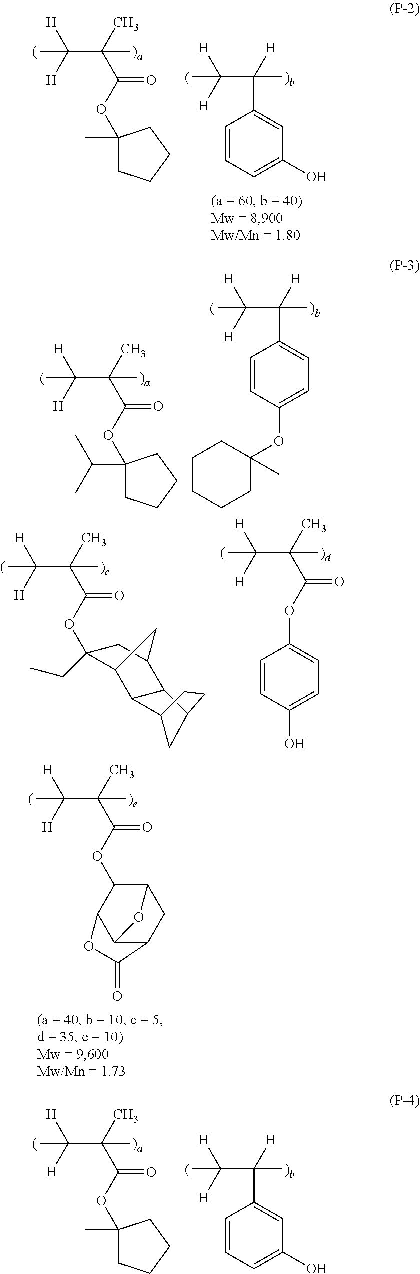

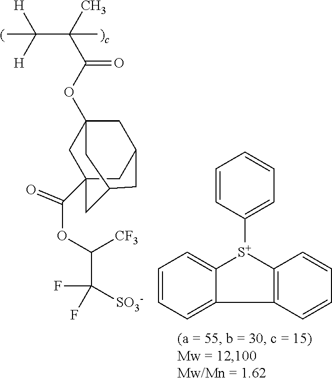

### Examples 2-1 to 2-46 and Comparative Examples 1-1 to 1-23

### Preparation of Resist Compositions

Resist compositions in solution form were prepared by dissolving an acid diffusion inhibitor (Sulfonium salts Q-1 to Q-18), a base polymer (Polymers P-1 to P-4), and optionally, a photoacid generator (PAG-1, PAG-2), an acid diffusion inhibitor other than the salt having formula (1) or (2), and an alkali-soluble surfactant (SF-1) in a solvent containing 0.01 wt % of surfactant PolyFox 636 (Omnova Solutions, Inc.), and filtering the solution through a Teflon® filter with a pore size of 0.2 μm. For comparison sake, resist compositions were similarly prepared using acid diffusion inhibitors Q-A to Q-I. The formulation of the thus prepared resist solutions is shown in Tables 1 to 3.

The photoacid generators PAG-1 and PAG-2, solvents, comparative acid diffusion inhibitors Q-A to Q-I, and alkali-soluble surfactant SF-1 in Tables 1 to 3 are identified below.

### Photoacid Generator: PAG-1 and PAG-2

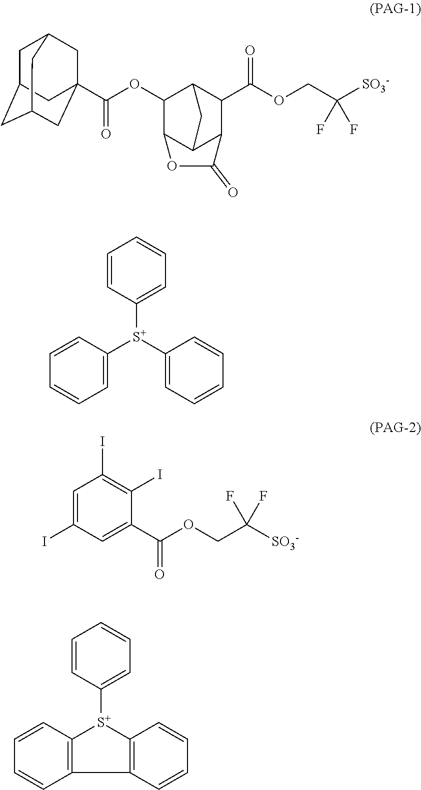

### Solvent:

PGMEA=propylene glycol monomethyl ether acetate

GBL=γ-butyrolactone

DAA=diacetone alcohol

### Acid Diffusion Inhibitor: Q-A to Q-I

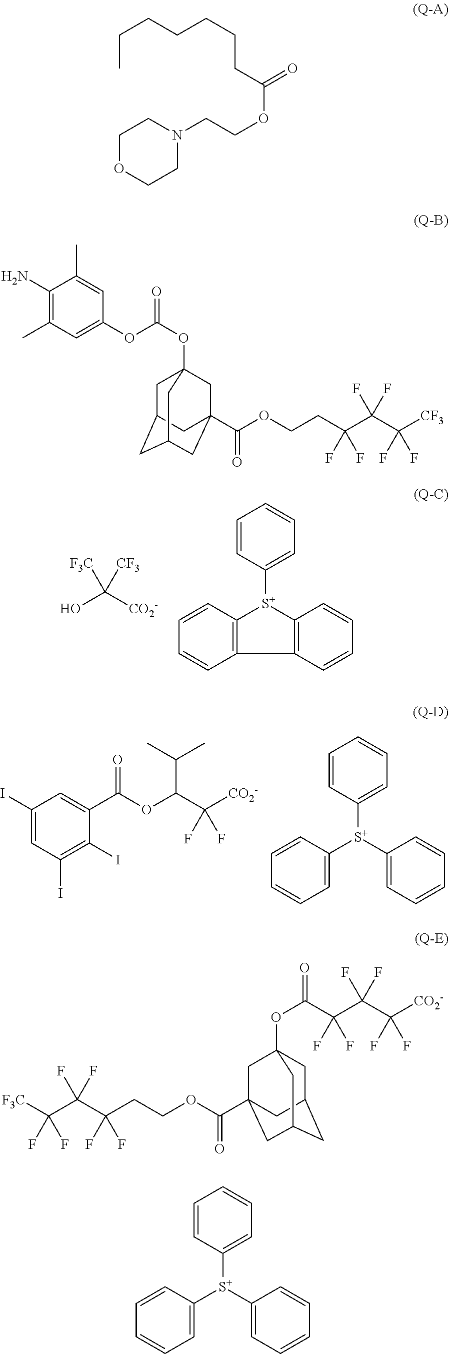

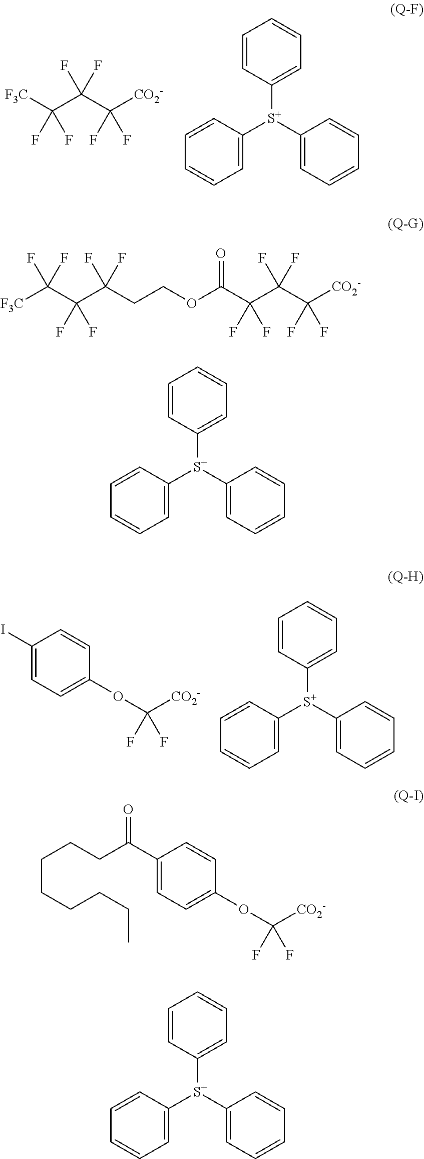

### Alkali-Soluble Surfactant SF-1:

poly(2,2,3,3,4,4,4-heptafluoro-1-isobutyl-1-butyl methacrylate/9-(2,2,2-trifluoro-1-trifluoromethylethyloxycarbonyl)-4-oxatricyclo[4.2.1.017]nonan-5-on-2-yl methacrylate)

Mw=7,700

Mw/Mn=1.82

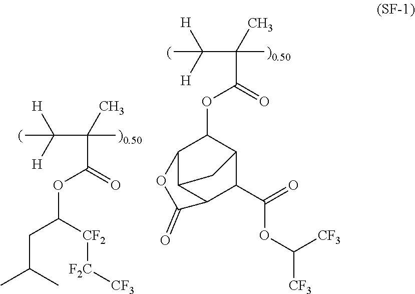

TABLE 1

| | | | | | Acid diffusion | | |
|---|---|---|---|---|---|---|---|
| | | Resist | Base polymer | Acid generator | inhibitor | Surfactant | Solvent |
| | | composition | (pbw) | (pbw) | (pbw) | (pbw) | (pbw) |
| Example | 2-1 | R-1 | P-1 | — | Q-1 | SF-1 | PGMEA/GBL |
| | | | (100) | | (4.6) | (3.0) | (1,920/480) |
| | 2-2 | R-2 | P-1 | — | Q-4 | SF-1 | PGMEA/GBL |
| | | | (100) | | (4.2) | (3.0) | (1,920/480) |
| | 2-3 | R-3 | p-1 | — | Q-7 | SF-1 | PGMEA/GBL |
| | | | (100) | | (4.2) | (3.0) | (1,920/480) |
| | 2-4 | R-4 | p-1 | — | Q-9 | SF-1 | PGMEA/GBL |
| | | | (100) | | (4.2) | (3.0) | (1,920/480) |
| | 2-5 | R-5 | p-1 | — | Q-14 | SF-1 | PGMEA/GBL |
| | | | (100) | | v(5.0) | (3.0) | (1,920/480) |
| | 2-6 | R-6 | p-1 | — | Q-18 | SF-1 | PGMEA/GBL |
| | | | (100) | | (5.0) | (3.0) | (1,920/480) |
| | 2-7 | R-7 | P-2 | PAG-1 | Q-1 | SF-1 | PGMEA/DAA |
| | | | (100) | (20.0) | (10.0) | (3.0) | (2,100/900) |
| | 2-8 | R-8 | P-2 | PAG-1 | Q-2 | SF-1 | PGMEA/DAA |
| | | | (100) | (20.0) | (10.0) | (3.0) | (2,100/900) |
| | 2-9 | R-9 | P-2 | PAG-1 | Q-3 | SF-1 | PGMEA/DAA |
| | | | (100) | (20.0) | (10.0) | (3.0) | (2,100/900) |
| | 2-10 | R-10 | P-2 | PAG-1 | Q-4 | SF-1 | PGMEA/DAA |
| | | | (100) | (20.0) | (9.0) | (3.0) | (2,100/900) |
| | 2-11 | R-11 | P-2 | PAG-1 | Q-5 | SF-1 | PGMEA/DAA |
| | | | (100) | (20.0) | (9.0) | (3.0) | (2,100/900) |
| | 2-12 | R-12 | P-2 | PAG-1 | Q-6 | SF-1 | PGMEA/DAA |
| | | | (100) | (20.0) | (9.0) | (3.0) | (2,100/900) |
| | 2-13 | R-13 | P-2 | PAG-1 | Q-7 | SF-1 | PGMEA/DAA |
| | | | (100) | (20.0) | (9.0) | (3.0) | (2,100/900) |
| | 2-14 | R-14 | P-2 | PAG-1 | Q-8 | SF-1 | PGMEA/DAA |
| | | | (100) | (20.0) | (10.0) | (3.0) | (2,100/900) |
| | 2-15 | R-15 | P-2 | PAG-1 | Q-9 | SF-1 | PGMEA/DAA |
| | | | (100) | (20.0) | (9.0) | (3.0) | (2,100/900) |
| | 2-16 | R-16 | P-2 | PAG-1 | Q-10 | SF-1 | PGMEA/DAA |
| | | | (100) | (20.0) | (10.0) | (3.0) | (2,100/900) |
| | 2-17 | R-17 | P-2 | PAG-1 | Q-11 | SF-1 | PGMEA/DAA |
| | | | (100) | (20.0) | (9.0) | (3.0) | (2,100/900) |
| | 2-18 | R-18 | P-2 | PAG-1 | Q-12 | SF-1 | PGMEA/DAA |
| | | | (100) | (20.0) | (9.5) | (3.0) | (2,100/900) |
| | 2-19 | R-19 | P-2 | PAG-1 | Q-13 | SF-1 | PGMEA/DAA |
| | | | (100) | (20.0) | (10.0) | (3.0) | (2,100/900) |
| | 2-20 | R-20 | P-2 | PAG-1 | Q-14 | SF-1 | PGMEA/DAA |
| | | | (100) | (20.0) | (12.0) | (3.0) | (2,100/900) |
| | 2-21 | R-21 | P-2 | PAG-1 | Q-15 | SF-1 | PGMEA/DAA |
| | | | (100) | (20.0) | (12.5) | (3.0) | (2,100/900) |
| | 2-22 | R-22 | P-2 | PAG-1 | Q-16 | SF-1 | PGMEA/DAA |
| | | | (100) | (20.0) | (12.0) | (3.0) | (2,100/900) |
| | 2-23 | R-23 | P-2 | PAG-1 | Q-17 | SF-1 | PGMEA/DAA |
| | | | (100) | (20.0) | (12.0) | (3.0) | (2,100/900) |

TABLE 2

| | | | | | Acid diffusion | | |
|---|---|---|---|---|---|---|---|
| | | Resist | Base polymer | Acid generator | inhibitor | Surfactant | Solvent |
| | | composition | (pbw) | (pbw) | (pbw) | (pbw) | (pbw) |
| Example | 2-24 | R-24 | P-2 | PAG-1 | Q-18 | SF-1 | PGMEA/DAA |
| | | | (100) | (20.0) | (9.0) | (3.0) | (2,100/900) |
| | 2-25 | R-25 | P-2 | PAG-2 | Q-1 | SF-1 | PGMEA/DAA |
| | | | (100) | (25.0) | (12.5) | (3.0) | (2,100/900) |
| | 2-26 | R-26 | P-2 | PAG-2 | Q-2 | SF-1 | PGMEA/DAA |
| | | | (100) | (25.0) | (12.5) | (3.0) | (2,100/900) |
| | 2-27 | R-27 | P-2 | PAG-2 | Q-8 | SF-1 | PGMEA/DAA |
| | | | (100) | (25.0) | (12.5) | (3.0) | (2,100/900) |
| | 2-28 | R-28 | P-2 | PAG-2 | Q-15 | SF-1 | PGMEA/DAA |
| | | | (100) | (25.0) | (16.0) | (3.0) | (2,100/900) |
| | 2-29 | R-29 | P-2 | PAG-2 | Q-16 | SF-1 | PGMEA/DAA |
| | | | (100) | (25.0) | (16.0) | (3.0) | (2,100/900) |
| | 2-30 | R-30 | P-3 | PAG-2 | Q-1 (5.0) | SF-1 | PGMEA/DAA |
| | | | (100) | (20.0) | Q-C (6.0) | (3.0) | (2,100/900) |
| | 2-31 | R-31 | P-3 | PAG-2 | Q-2 (5.0) | SF-1 | PGMEA/DAA |
| | | | (100) | (20.0) | Q-C (6.0) | (3.0) | (2,100/900) |
| | 2-32 | R-32 | P-3 | PAG-2 | Q-8 (5.0) | SF-1 | PGMEA/DAA |
| | | | (100) | (20.0) | Q-C (6.0) | (3.0) | (2,100/900) |
| | 2-33 | R-33 | P-3 | PAG-2 | Q-13 (5.0) | SF-1 | PGMEA/DAA |
| | | | (100) | (20.0) | Q-C (6.0) | (3.0) | (2,100/900) |
| | 2-34 | R-34 | P-4 | — | Q-1 | SF-1 | PGMEA/DAA |
| | | | (100) | | (16.0) | (3.0) | (2,100/900) |
| | 2-35 | R-35 | P-4 | — | Q-2 | SF-1 | PGMEA/DAA |
| | | | (100) | | (16.0) | (3.0) | (2,100/900) |
| | 2-36 | R-36 | P-4 | — | Q-3 | SF-1 | PGMEA/DAA |
| | | | (100) | | (16.0) | (3.0) | (2,100/900) |
| | 2-37 | R-37 | P-4 | — | Q-6 | SF-1 | PGMEA/DAA |
| | | | (100) | | (14.5) | (3.0) | (2,100/900) |
| | 2-38 | R-38 | P-4 | — | Q-7 | SF-1 | PGMEA/DAA |
| | | | (100) | | (14.5) | (3.0) | (2,100/900) |
| | 2-39 | R-39 | P-4 | — | Q-8 | SF-1 | PGMEA/DAA |
| | | | (100) | | (16.0) | (3.0) | (2,100/900) |
| | 2-40 | R-40 | P-4 | — | Q-13 | SF-1 | PGMEA/DAA |
| | | | (100) | | (16.0) | (3.0) | (2,100/900) |
| | 2-41 | R-41 | P-4 | — | Q-15 | SF-1 | PGMEA/DAA |
| | | | (100) | | (18.0) | (3.0) | (2,100/900) |
| | 2-42 | R-42 | P-4 | — | Q-16 | SF-1 | PGMEA/DAA |
| | | | (100) | | (18.0) | (3.0) | (2,100/900) |
| | 2-43 | R-43 | P-4 | — | Q-18 | SF-1 | PGMEA/DAA |
| | | | (100) | | (18.0) | (3.0) | (2,100/900) |
| | 2-44 | R-44 | P-4 | — | Q-1 (4.0) | SF-1 | PGMEA/DAA |
| | | | (100) | | Q-D (12.0) | (3.0) | (2,100/900) |
| | 2-45 | R-45 | P-4 | — | Q-2 (4.0) | SF-1 | PGMEA/DAA |
| | | | (100) | | Q-D (12.0) | (3.0) | (2,100/900) |
| | 2-46 | R-46 | P-4 | — | Q-15 (6.0) | SF-1 | PGMEA/DAA |
| | | | (100) | | Q-D (12.0) | (3.0) | (2,100/900) |

TABLE 3

| | | | | | Acid diffusion | | |
|---|---|---|---|---|---|---|---|
| | | Resist | Base polymer | Acid generator | inhibitor | Surfactant | Solvent |
| | | composition | (pbw) | (pbw) | (pbw) | (pbw) | (pbw) |
| Comparative | 1-1 | CR-1 | P-1 | — | Q-A | SF-1 | PGMEA/GBL |
| Example | | | (100) | | (2.8) | (3.0) | (1,920/480) |
| | 1-2 | CR-2 | P-1 | — | Q-B | SF-1 | PGMEA/GBL |
| | | | (100) | | (2.8) | (3.0) | (1,920/480) |
| | 1-3 | CR-3 | p-1 | — | Q-E | SF-1 | PGMEA/GBL |
| | | | (100) | | (5.0) | (3.0) | (1,920/480) |
| | 1-4 | CR-4 | p-1 | — | Q-F | SF-1 | PGMEA/GBL |
| | | | (100) | | (5.0) | (3.0) | (1,920/480) |
| | 1-5 | CR-5 | p-1 | — | Q-G | SF-1 | PGMEA/GBL |
| | | | (100) | | (5.0) | (3.0) | (1,920/480) |
| | 1-6 | CR-6 | p-1 | — | Q-I | SF-1 | PGMEA/GBL |
| | | | (100) | | (5.0) | (3.0) | (1,920/480) |
| | 1-7 | CR-7 | P-2 | PAG-1 | Q-A | SF-1 | PGMEA/DAA |
| | | | (100) | (20.0) | (6.0) | (3.0) | (2,100/900) |
| | 1-8 | CR-8 | P-2 | PAG-1 | Q-B | SF-1 | PGMEA/DAA |
| | | | (100) | (20.0) | (10.0) | (3.0) | (2,100/900) |
| | 1-9 | CR-9 | P-2 | PAG-1 | Q-E | SF-1 | PGMEA/DAA |
| | | | (100) | (20.0) | (10.0) | (3.0) | (2,100/900) |
| | 1-10 | CR-10 | P-2 | PAG-1 | Q-F | SF-1 | PGMEA/DAA |
| | | | (100) | (20.0) | (10.0) | (3.0) | (2,100/900) |
| | 1-11 | CR-11 | P-2 | PAG-1 | Q-G | SF-1 | PGMEA/DAA |
| | | | (100) | (20.0) | (10.0) | (3.0) | (2,100/900) |
| | 1-12 | CR-12 | P-2 | PAG-1 | Q-H | SF-1 | PGMEA/DAA |
| | | | (100) | (20.0) | (10.0) | (3.0) | (2,100/900) |
| | 1-13 | CR-13 | P-2 | PAG-1 | Q-I | SF-1 | PGMEA/DAA |
| | | | (100) | (20.0) | (10.0) | (3.0) | (2,100/900) |
| | 1-14 | CR-14 | P-3 | PAG-2 | Q-E (3.8) | SF-1 | PGMEA/DAA |
| | | | (100) | (20.0) | Q-C (6.0) | (3.0) | (2,100/900) |
| | 1-15 | CR-15 | P-3 | PAG-2 | Q-I (3.8) | SF-1 | PGMEA/DAA |
| | | | (100) | (20.0) | Q-C (6.0) | (3.0) | (2,100/900) |
| | 1-16 | CR-16 | P-4 | — | Q-A | SF-1 | PGMEA/DAA |
| | | | (100) | | (8.0) | (3.0) | (2,100/900) |
| | 1-17 | CR-17 | P-4 | — | Q-B | SF-1 | PGMEA/DAA |
| | | | (100) | | (8.0) | (3.0) | (2,100/900) |
| | 1-18 | CR-18 | P-4 | — | Q-E | SF-1 | PGMEA/DAA |
| | | | (100) | | (16.0) | (3.0) | (2,100/900) |
| | 1-19 | CR-19 | P-4 | — | Q-G | SF-1 | PGMEA/DAA |
| | | | (100) | | (16.0) | (3.0) | (2,100/900) |
| | 1-20 | CR-20 | P-4 | — | Q-H | SF-1 | PGMEA/DAA |
| | | | (100) | | (16.0) | (3.0) | (2,100/900) |
| | 1-21 | CR-21 | P-4 | — | Q-I | SF-1 | PGMEA/DAA |
| | | | (100) | | (16.0) | (3.0) | (2,100/900) |
| | 1-22 | CR-22 | P-4 | — | Q-B (2.0) | SF-1 | PGMEA/DAA |
| | | | (100) | | Q-D (12.0) | (3.0) | (2,100/900) |
| | 1-23 | CR-23 | P-4 | — | Q-E (4.0) | SF-1 | PGMEA/DAA |
| | | | (100) | | Q-D (12.0) | (3.0) | (2,100/900) |

### Examples 3-1 to 3-6 and Comparative Examples 2-1 to 2-6

### ArF Immersion Lithography Patterning Test

On a silicon substrate, an antireflective coating solution (ARC-29A by Nissan Chemical Corp.) was coated and baked at 180° C. for 60 seconds to form an ARC of 100 nm thick. On the ARC, each of the resist compositions (R-1 to R-6, CR-1 to CR-6) was spin coated and baked on a hotplate at 100° C. for 60 seconds to form a resist film of 90 nm thick.

Using an ArF excimer laser scanner (NSR-S610C by Nikon Corp., NA 1.30, σ 0.94/0.74, dipole 35 deg. illumination, 6% halftone phase shift mask), the resist film was exposed by the immersion lithography. Water was used as the immersion liquid. After exposure, the resist film was baked (PEB) at 90° C. for 60 seconds and developed in 2.38 wt % TMAH aqueous solution for 60 seconds to form a line-and-space (LS) pattern.

The LS pattern as developed was observed under CD-SEM (CG-5000 by Hitachi High-Technologies Corp.) and evaluated for sensitivity and LWR by the following methods. The results are shown in Table 4.

### Evaluation of Sensitivity

The optimum dose (Eop) is a dose (mJ/cm²) which provides a LS pattern having a line width of 40 nm at a pitch of 80 nm and reported as sensitivity. A smaller value indicates a higher sensitivity.

### Evaluation of LWR

On the L/S pattern formed by exposure in the optimum dose Eop, the line width was measured at longitudinally spaced apart 10 points, from which a 3-fold value (36) of the standard deviation (6) was determined and reported as LWR. A smaller value of 36 indicates a pattern having a lower roughness and more uniform line width. A pattern with a LWR value of 2.5 nm or less is rated “Good” while a pattern with a LWR value in excess of 2.5 nm is rated “NG.”

TABLE 4

| | | Resist | Eop | LWR |
|---|---|---|---|---|
| | | composition | (mJ/cm²) | (nm) |
| Example | 3-1 | R-1 | 41 | Good (2.3) |
| | 3-2 | R-2 | 40 | Good (2.2) |
| | 3-3 | R-3 | 43 | Good (2.3) |
| | 3-4 | R-4 | 39 | Good (2.4) |
| | 3-5 | R-5 | 39 | Good (2.4) |
| | 3-6 | R-6 | 41 | Good (2.3) |
| Comparative | 2-1 | CR-1 | 42 | NG (3.0) |
| Example | 2-2 | CR-2 | 39 | NG (2.8) |
| | 2-3 | CR-3 | 38 | NG (2.8) |
| | 2-4 | CR-4 | 42 | NG (2.9) |
| | 2-5 | CR-5 | 42 | NG (2.8) |
| | 2-6 | CR-6 | 40 | NG (2.7) |

As is evident from Table 4, the resist compositions within the scope of the invention exhibit satisfactory LWR and are useful as the ArF immersion lithography material.

### Examples 4-1 to 4-40 and Comparative Examples 3-1 to 3-17

### EUV Lithography Test

Each of the resist compositions (R-7 to R-46, CR-7 to CR-23) was spin coated on a silicon substrate having a 20-nm coating of silicon-containing spin-on hard mask SHB-A940 (silicon content 43 wt %, Shin-Etsu Chemical Co., Ltd.) and prebaked on a hotplate at 100° C. for 60 seconds to form a resist film of 50 nm thick. Using an EUV scanner NXE3400 (ASML, NA 0.33, σ 0.9/0.6, quadrupole illumination), the resist film was exposed to EUV through a mask bearing a hole pattern having a pitch 46 nm +20% bias (on-wafer size). The resist film was baked (PEB) on a hotplate at 85° C. for 60 seconds and developed in a 2.38 wt % TMAH aqueous solution for 30 seconds to form a hole pattern having a size of 23 nm.

The hole pattern as developed was observed under CD-SEM (CG-6300 by Hitachi High-Technologies Corp.) and evaluated for sensitivity and CDU by the following methods. The results are shown in Tables 5 to 7.

### Evaluation of Sensitivity

The optimum dose (Eop) is a dose (mJ/cm²) which provides a hole pattern having a hole size of 23 nm and reported as sensitivity. A smaller value indicates a higher sensitivity.

### Evaluation of CDU

For the hole pattern at the optimum dose (Eop), the size of 50 holes within the same dose shot was measured, from which a 3-fold value (36) of the standard deviation (6) was computed and reported as CDU. A smaller value of CDU indicates a hole pattern of better dimensional uniformity. The sample was rated “Good” for a CDU value of up to 3.0 nm and “NG” for a CDU value in excess of 3.0 nm.

TABLE 5

| | | Resist | Eop | CDU |
|---|---|---|---|---|
| | | composition | (mJ/cm²) | (nm) |
| Example | 4-1 | R-7 | 31 | Good (2.7) |
| | 4-2 | R-8 | 31 | Good (2.7) |
| | 4-3 | R-9 | 32 | Good (2.8) |
| | 4-4 | R-10 | 33 | Good (2.8) |
| | 4-5 | R-11 | 34 | Good (2.8) |
| | 4-6 | R-12 | 34 | Good (2.7) |
| | 4-7 | R-13 | 33 | Good (2.9) |
| | 4-8 | R-14 | 31 | Good (2.7) |
| | 4-9 | R-15 | 34 | Good (2.7) |
| | 4-10 | R-16 | 33 | Good (2.8) |
| | 4-11 | R-17 | 34 | Good (2.9) |
| | 4-12 | R-18 | 33 | Good (2.8) |
| | 4-13 | R-19 | 32 | Good (2.8) |
| | 4-14 | R-20 | 32 | Good (2.7) |
| | 4-15 | R-21 | 30 | Good (2.7) |
| | 4-16 | R-22 | 31 | Good (2.6) |
| | 4-17 | R-23 | 30 | Good (2.8) |
| | 4-18 | R-24 | 33 | Good (2.6) |
| | 4-19 | R-25 | 30 | Good (2.9) |
| | 4-20 | R-26 | 30 | Good (2.7) |

TABLE 6

| | | Resist | Eop | CDU |
|---|---|---|---|---|
| | | composition | (mJ/cm²) | (nm) |
| Example | 4-21 | R-27 | 29 | Good (2.8) |
| | 4-22 | R-28 | 30 | Good (3.0) |
| | 4-23 | R-29 | 31 | Good (2.8) |
| | 4-24 | R-30 | 31 | Good (2.8) |
| | 4-25 | R-31 | 31 | Good (2.6) |
| | 4-26 | R-32 | 32 | Good (2.7) |
| | 4-27 | R-33 | 31 | Good (2.9) |
| | 4-28 | R-34 | 29 | Good (2.7) |
| | 4-29 | R-35 | 29 | Good (2.6) |
| | 4-30 | R-36 | 29 | Good (2.5) |
| | 4-31 | R-37 | 32 | Good (2.8) |
| | 4-32 | R-38 | 33 | Good (2.6) |
| | 4-33 | R-39 | 30 | Good (2.6) |
| | 4-34 | R-40 | 30 | Good (2.5) |
| | 4-35 | R-41 | 29 | Good (2.7) |
| | 4-36 | R-42 | 31 | Good (2.7) |
| | 4-37 | R-43 | 29 | Good (2.6) |
| | 4-38 | R-44 | 28 | Good (2.4) |
| | 4-39 | R-45 | 30 | Good (2.6) |
| | 4-40 | R-46 | 29 | Good (2.5) |

TABLE 7

| | | Resist | Eop | CDU |
|---|---|---|---|---|
| | | composition | (mJ/cm²) | (nm) |
| Comparative | 3-1 | CR-7 | 36 | NG (3.6) |
| Example | 3-2 | CR-8 | 37 | NG (3.4) |
| | 3-3 | CR-9 | 31 | NG (3.2) |
| | 3-4 | CR-10 | 32 | NG (3.4) |
| | 3-5 | CR-11 | 32 | NG (3.3) |
| | 3-6 | CR-12 | 32 | NG (3.1) |
| | 3-7 | CR-13 | 31 | NG (3.2) |
| | 3-8 | CR-14 | 32 | NG (3.3) |
| | 3-9 | CR-15 | 32 | NG (3.2) |
| | 3-10 | CR-16 | 34 | NG (3.3) |
| | 3-11 | CR-17 | 35 | NG (3.3) |
| | 3-12 | CR-18 | 31 | NG (3.2) |
| | 3-13 | CR-19 | 29 | NG (3.3) |
| | 3-14 | CR-20 | 31 | NG (3.1) |
| | 3-15 | CR-21 | 30 | NG (3.1) |
| | 3-16 | CR-22 | 32 | NG (3.3) |
| | 3-17 | CR-23 | 30 | NG (3.2) |

As is evident from Tables 5 to 7, the resist compositions within the scope of the invention exhibit satisfactory values of CDU and are useful as the EUV lithography material.

Japanese Patent Application No. 2021-212355 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A salt compound having the formula (1) or (2): 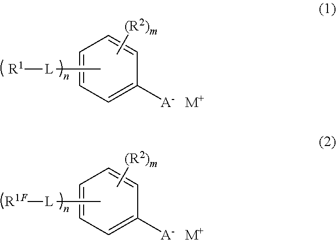
wherein n is an integer of 1 to 5, m is an integer of 0 to 4,
L is a single bond, ether bond or ester bond, L may be the same or different when n is 2 or more,
R¹ is a C₆-C₁₈ alkyl group in which some constituent —CH₂— may be replaced by an ether bond or carbonyl moiety, R¹ has at least one straight structure of 6 or more carbon atoms, R¹ may be the same or different when n is 2 or more, the alkyl group may contain a cyclic structure selected from cyclopentane, cyclohexane, adamantane, norbornane and benzene rings as a partial structure at its end or in its carbon-carbon bond,
R^{1F} is a C₄-C₁₈ fluorinated alkyl group in which some constituent —CH₂— may be replaced by an ether bond or carbonyl moiety, R^{1F} has at least two groups selected from —CF₂— and —CF₃, the fluorinated alkyl group may contain a cyclic structure selected from cyclopentane, cyclohexane, adamantane, norbornane and benzene rings as a partial structure at its end or in its carbon-carbon bond,
R² is halogen, hydroxy, or a C₁-C₁₀ hydrocarbyl group in which some hydrogen may be substituted by halogen and some constituent —CH₂— may be replaced by an ether bond or carbonyl moiety,
M⁺ is a sulfonium or iodonium cation,
A⁻ is an anion having any one of the formulae (A1) to (A4), with the proviso that the partial structure R¹-L- or R^{1F}-L- in formula (1) or (2) is bonded to the benzene ring via —CH₂— or —O— when A⁻ is an anion having formula (A2), 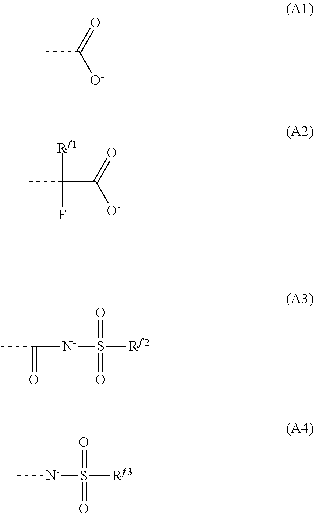
wherein R^{f1} is hydrogen or fluorine, R^{f2} and R^{f3} are each independently methyl, phenyl, tolyl or a C₁-C₄ perfluoroalkyl group, the broken line designates a valence bond.

2. The salt compound of claim 1 wherein A⁻ is an anion having formula (A1) or (A2).

3. The salt compound of claim 1 wherein m is 1 or more and at least one R² is iodine.

4. The salt compound of claim 1 wherein M⁺ is a cation having any one of the formulae (M-1) to (M-3): 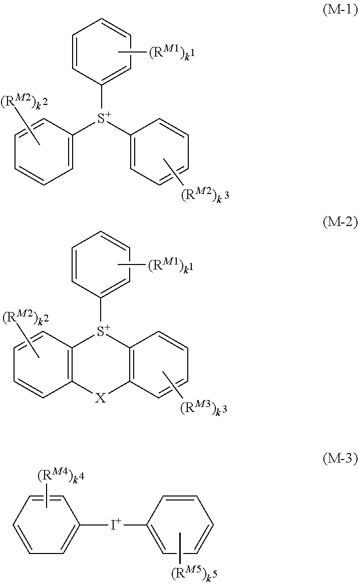
wherein R^{M1}, R^{M2}, R^{M3}, R^{M4} and R^{M5} are each independently hydroxy, halogen, or a C₁-C₁₅ hydrocarbyl group in which some or all of the hydrogen atoms may be substituted by a heteroatom-containing moiety and some constituent —CH₂— may be replaced by —O—, —C(═O)—, —S—, —S(═O)—, —S(═O)₂— or —N(H)—,
k¹, k², k³, k⁴ and k⁵ are each independently an integer of 0 to 5, when k¹ is 2 or more, R^{M1} may be the same or different and two R^{M1} may bond together to form a ring with the carbon atoms on the benzene ring to which they are attached, when k² is 2 or more, R^{M2} may be the same or different and two R^{M2} may bond together to form a ring with the carbon atoms on the benzene ring to which they are attached, when k³ is 2 or more, R^{M3} may be the same or different and two R^{M3} may bond together to form a ring with the carbon atoms on the benzene ring to which they are attached, when k⁴ is 2 or more, R^{M4} may be the same or different and two R^{M4} may bond together to form a ring with the carbon atoms on the benzene ring to which they are attached, when k⁵ is 2 or more, R^{M5} may be the same or different and two R^{M5} may bond together to form a ring with the carbon atoms on the benzene ring to which they are attached, and
X is a single bond, —CH₂—, —O—, —C(═O)—, —S—, —S(═O)—, —S(═O)₂— or —N(H)—.

5. The salt compound of claim 4, consisting of an anion having the formula (1-I) or (2-I) and a cation having formula (M-1) or (M-2): 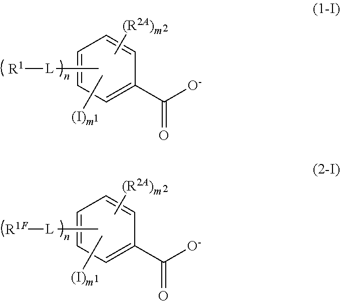
wherein L, R¹, R^{1F} and n are as defined above,
R^{2A} is halogen exclusive of iodine, hydroxy, or a C₁-C₁₀ hydrocarbyl group in which some hydrogen may be substituted by halogen and some constituent —CH₂— may be replaced by an ether bond or carbonyl moiety,
m¹ is an integer of 1 to 4, m² is an integer of 0 to 3, and n+m¹+m² is from 2 to 5.

6. An acid diffusion inhibitor comprising the salt compound of claim 1.

7. A resist composition comprising (A) a base polymer adapted to change its solubility in a developer under the action of an acid, (B) a photoacid generator, (C) the acid diffusion inhibitor of claim 6, and (D) an organic solvent.

8. A resist composition comprising (A′) a base polymer adapted to change its solubility in a developer under the action of an acid, the base polymer comprising a photoacid generating site having a function of generating an acid upon exposure to radiation, (C) the acid diffusion inhibitor of claim 6, and (D) an organic solvent.

9. The resist composition of claim 7 wherein the base polymer is a polymer comprising repeat units having the formula (a) or repeat units having the formula (b): 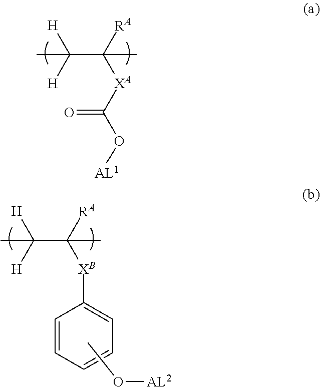
wherein R^{A} is hydrogen or methyl,
X^{A} is a single bond, phenylene, naphthylene, or *—C(═O)—O—X^{A1}—, X^{A1} is a C₁-C₁₅ hydrocarbylene group which may contain at least one moiety selected from hydroxy, ether bond, ester bond, and lactone ring, * designates a point of attachment to the carbon atom in the backbone,
X^{B} is a single bond or ester bond, and
AL¹ and AL² are each independently an acid labile group.

10. The resist composition of claim 9 wherein the acid labile group has the formula (L1): 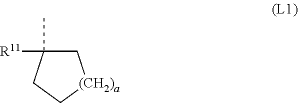
wherein R¹¹ is a C₁-C₇ hydrocarbyl group in which some constituent —CH₂— may be replaced by —O—, a is 1 or 2, and the broken line designates a valence bond.

11. The resist composition of claim 7 wherein the base polymer comprises repeat units having the formula (c): 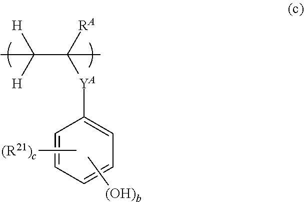
wherein R^{A} is hydrogen or methyl, Y^{A} is a single bond or ester bond, R²¹ is fluorine, iodine or a C₁-C₁₀ hydrocarbyl group in which some constituent —CH₂— may be replaced by —O— or —C(═O)—, b is an integer of 1 to 5, c is an integer of 0 to 4, and b+c is from 1 to 5.

12. The resist composition of claim 8 wherein the base polymer comprises repeat units of at least one type selected from the formulae (d1) to (d4): 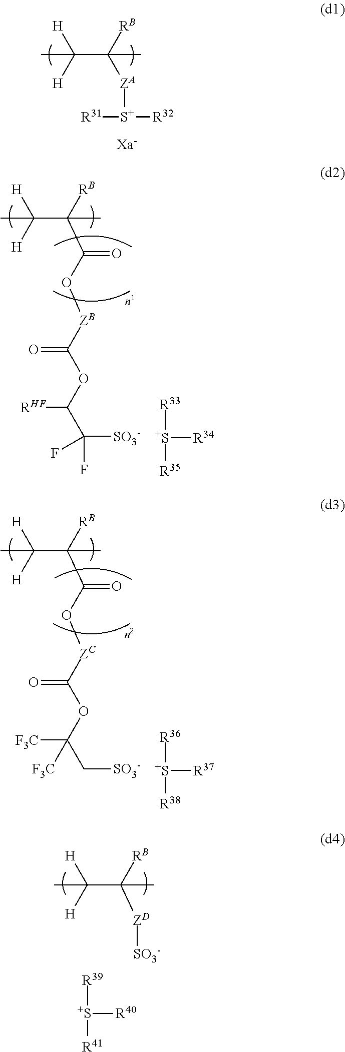
wherein R^{B} is hydrogen, fluorine, methyl or trifluoromethyl,
Z^{A} is a single bond, phenylene group, —O—Z^{A1}—, —C(═O)—O—Z^{A1}— or —C(═O)—N(H)—Z^{A1}—, Z^{A1} is a C₁-C₂₀ hydrocarbylene group which may contain a heteroatom,
Z^{B} and Z^{C} are each independently a single bond or a C₁-C₂₀ hydrocarbylene group which may contain a heteroatom,
Z^{D} is a single bond, methylene, ethylene, phenylene, fluorinated phenylene, —O—Z^{D1}—, —C(═O)—O—Z^{D1}— or —C(═O)—N(H)—Z^{D1}—, Z^{D1} is an optionally substituted phenylene group,
R³¹ to R⁴¹ are each independently a C₁-C₂₀ hydrocarbyl group which may contain a heteroatom, any two or more of Z^{A}, R³¹ and R³² may bond together to form a ring with the sulfur atom to which they are attached, any two of R³³, R³⁴ and R³⁵, any two or more of R³⁶, R³⁷ and R³⁸, and any two or more of R³⁹, R⁴⁰ and R⁴¹ may bond together to form a ring with the sulfur atom to which they are attached,
R^{HF} is hydrogen or trifluoromethyl,
n¹ is 0 or 1, n¹ is 0 when Z^{B} is a single bond, n² is 0 or 1, n² is 0 when Z^{C} is a single bond, and
Xa⁻ is a non-nucleophilic counter ion.

13. A pattern forming process comprising the steps of applying the resist composition of claim 7 onto a substrate to form a resist film thereon, exposing a selected region of the resist film to KrF excimer laser, ArF excimer laser, EB or EUV, and developing the exposed resist film in a developer.

14. The pattern forming process of claim 13 wherein the developing step uses an alkaline aqueous solution as the developer, thereby forming a positive pattern in which an exposed region of the resist film is dissolved away and an unexposed region of the resist film is not dissolved.

15. The pattern forming process of claim 13 wherein the developing step uses an organic solvent as the developer, thereby forming a negative pattern in which an unexposed region of the resist film is dissolved away and an exposed region of the resist film is not dissolved.

16. The pattern forming process of claim 15 wherein the organic solvent is at least one solvent selected from the group consisting of 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate.
